(19) United States
(12) Patent Application Publication
(10) Pub. No.: US 2018/0221610 A1
(43) Pub. Date: Aug. 9, 2018

(54) Systems, Methods, and Devices for Facilitating Endotracheal Intubation

(71) Applicant: Intuvate, Inc., Mountain View, CA (US)

(72) Inventors: Barrett J. Larson, Mountain View, CA (US); Elizabeth Zambricki, Palo Alto, CA (US); Benjamin Ari Lubkin, San Francisco, CA (US)

(73) Assignee: Intuvate, Inc., Mountain View, CA (US)

(21) Appl. No.: 15/877,370

(22) Filed: Jan. 22, 2018

Related U.S. Application Data
(63) Continuation-in-part of application No. 14/714,189, filed on May 15, 2015, now abandoned.
(60) Provisional application No. 62/448,876, filed on Jan. 20, 2017.
(60) Provisional application No. 62/535,462, filed on Jul. 21, 2017.
(60) Provisional application No. 62/117,461, filed on Feb. 18, 2015.
(60) Provisional application No. 62/104,682, filed on Jan. 16, 2015.
(60) Provisional application No. 61/993,275, filed on May 15, 2014.

(51) Int. Cl. A61M 16/04; G06T 7/73; H04N 7/18; A61B 5/06; A61B 1/267; A61B 1/05; A61B 1/00; A61B 34/20

(52) U.S. Cl. CPC A61M 16/0488; G06T 7/74; H04N 7/183; A61B 5/062; A61B 1/267; A61B 2034/2057; A61B 1/00048; A61B 34/20; G06T 2207/10016; A61B 1/00117; A61B 1/05

(57) ABSTRACT

A system for facilitating an endotracheal intubation may include a video laryngoscope (VL) apparatus and an intubation device guidance system integrate with or separate from the VL apparatus. The VL apparatus may include a VL body and a video camera. The intubation device guidance system may include one or more magnetometers arranged relative to the video laryngoscope body and configured to generate magnetometer signals based on an interaction with one or more magnetic elements associated with an intubation device. The intubation device guidance system may also include a processor communicatively coupled to the one or more magnetometers and configured to calculate position information regarding the intubation device relative to the video laryngoscope apparatus based on the magnetometer signals. An information output device may be provided to output information to a user indicating or based on the calculated position information regarding the intubation device.

### TECHNICAL FIELD OF THE INVENTION

The present disclosure relates generally to the field of airway management, and more specifically to systems, methods and devices for detecting, monitoring, and providing feedback regarding the position of an endotracheal tube (e.g., via detection of a stylet within the endotracheal tube), e.g., to facilitate an intubation procedure.

### BACKGROUND

Increasingly, video cameras are being incorporated into medical practice to facilitate diagnosis and treatment procedures. In medicine, video cameras are often used to allow providers to view internal body structures that would otherwise not be directly visible. While video technology has improved the access, efficacy, safety, and precision of many procedures, optical imaging techniques do have some limitations. For example, video cameras have a relatively limited field of view and the camera lens has the potential to be obscured by fluid, blood, gastric contents, mucous, etc. Furthermore, when procedural instruments are outside of a video camera's field of view, they are effectively in a “blind spot” and the proceduralist may be forced to navigate the instrument without any optical guidance. Blind manipulation of instruments imparts an inherent safety risk to the patient as the instrument has the capacity to damage unseen anatomic structures or collide with other instruments that are also outside of the camera's field of view.

Airway management commonly employs videographic and fiberoptic imaging techniques. Intubation is the process wherein an endotracheal tube is inserted into the patient's airway to enable mechanical ventilation. Direct laryngoscopy is the conventional method of intubation, wherein the proceduralist use a laryngoscope to obtain a direct line-of-sight view of the glottis, vocal cords, and trachea. Drawbacks of conventional direct laryngoscopy include extension of the neck, which may be contraindicated in the setting of known or suspected neck injury. Additionally, certain variations in neck anatomy such as large neck circumference or anteriorly placed airways may make direct visualization of the vocal cords more difficult or impossible using traditional direct laryngoscopy methods.

Indirect, or video laryngoscopy, is an alternative intubation technique. Video laryngoscopy typically utilizes a camera placed at the tip of a laryngoscope blade, allowing the proceduralist to indirectly view an image of the glottis and vocal cords. This camera is capable of obtaining anatomic views that are often not possible with direct line-of-sight techniques. Video laryngoscopes offer specific advantages over traditional laryngoscopes. For example, video laryngoscopes offer improved rates of successful intubation. Difficult anatomy, such as an anterior airway, can be visualized more easily and with less neck manipulation using a video laryngoscope. Video laryngoscopy also requires less training given the shorter learning curve. While video laryngoscopy offers several benefits, one of the main drawbacks is that the video laryngoscope contains “blind spots” wherein the operator is unable to visualize the endotracheal tube tip until it appears in front of the video laryngoscope camera lens.

Several studies have shown that while video laryngoscopy offers increased efficacy with first attempt intubations as well as a reduction in time to intubate, the complication rate associated with video laryngoscopy is equal to or exceeds that of direct laryngoscopy. Common complications associated with video laryngoscopy are due to the “blind spot” encountered between the introduction of the endotracheal tube into the mouth and the visualization of the endotracheal tube by the video laryngoscope camera. These complications can include trauma to the oral cavity and oropharynx, and can be severe, requiring additional surgical intervention. Several case reports highlight significant video laryngoscope-related complications, including piercing of the soft palate, perforation of the tonsillar pillar, dissection of the palatopharyngeal arch, and damage to the retromolar trigone. In one case, the endotracheal stylet was described as transforming into “sharp knife-like weapon that cut through the patient's oral tissues”. While the video laryngoscope provides an excellent image of the vocal cords, the path of the endotracheal tube from the mouth to the vocal cords contains a blind spot wherein the operator cannot see the endotracheal tube, and thus trauma can occur.

Recommendations for decreasing risk of airway trauma when using a video laryngoscope include keeping the endotracheal tube as close as possible to the side of the laryngoscope blade, with the beveled tip facing against the blade. Many video laryngoscope manufacturers highlight the importance of this technique in order to minimize the risk of airway trauma. Despite the emphasis on keeping the endotracheal tube proximate the laryngoscope, studies have shown that adherence to this technique is suboptimal.

In addition to the inherent blind spot that exists with video laryngoscopy, if the airway has been compromised by blood, secretions, or gastric contents, it may be difficult or impossible to visualize the vocal cords using any optical means. In these situations, intubation can be facilitated by using non-optical (i.e. without airway visualization) techniques to guide an endotracheal tube into the trachea. The “Light Wand” is an example of a non-optical intubation technique, which involves using a lighted stylet, such that the light can be seen through the tissues of the anterior neck (transillumination). By examining the character of the transillumination (brightness, density, location, etc.), the lighted stylet can be guided into the trachea without ever directly visualizing the airway. Successful insertion of the stylet into the trachea is represented by a characteristic light pattern that is visible through the tissues of the anterior neck. Given that the transillumination technique does not require visualization of the airway, the technique can be useful when the airway has been compromised by blood or fluid. However, the transillumination technique does have some limitations, which include the requirement for a dark or dimly lit environment, which is often not feasible in an emergency situation or outdoors. Transillumination can also be difficult in obese patients where there is a large amount of soft tissue in the anterior neck or in thin patients where the pre-tracheal glow may be seen even with an esophageal intubation. Transillumination can also be difficult in patients with darker skin tones.

As such, in the event that the view of the airway is obscured, the need for an alternative method of non-optical tracheal intubation exists, particularly in the emergency room or pre-hospital setting.

### SUMMARY

Described herein are intubation guidance systems, methods, and devices for determining and monitoring position information regarding devices used during an intubation procedure, referred to herein as medical devices, and providing intubation guidance information to a user, e.g., a proceduralist performing an intubation, regarding the determined position information, to facilitate the intubation procedure. Some embodiments are integrated with or otherwise configured for use in conjunction with a video laryngoscope (“VL”), to thereby provide improved VL-assisted intubation systems and techniques.

“Position information” may include, for example, the location, orientation, velocity, and/or other positional or movement related parameters of one or more medical devices. Some embodiments may be configured to determine position information of one or more medical devices relative to an anatomical landmark (e.g., the trachea or vocal cords) or relative to one or more other medical devices. For example, as discussed in detail below, in some embodiments an intubation guidance system may determine the location and angular orientation of an endotracheal tube stylet relative to a video laryngoscope inserted in a patient's oral cavity and/or relative to the patient's trachea or other anatomical landmark.

“Position information” may also include information indicating whether a detectable element (e.g., a specified portion of the device or point on a monitored medical device) has reached a specified position or crossed a specified plane. For example, position information regarding a monitored ETT stylet may include information indicating whether a specified portion or point on the ETT stylet (e.g., corresponding with a magnetized portion at the distal tip of the stylet) has crossed a defined “camera plane” that defines a camera-viewable region in space that is visible to the VL camera (e.g., an area downstream of the camera) and a camera-hidden region in space that is not visible to the VL camera (e.g., an area upstream of the camera). In some embodiments an intubation guidance system may utilize this position information to identify “camera plane crossing” events (e.g., including insertion-direction camera plane crossing events and/or withdrawal-direction camera plane crossing events) and automatically trigger one or more functions or actions in response to such events, for example, automatically displaying a notification visible by the proceduralist (e.g., via colored LED(s) on or near the VL handle) that indicates the proceduralist may switch their attention from the patient's mouth to a VL video monitor displaying the VL camera view, and vice versa.

“Intubation guidance information” or simply “guidance information” may include any information detectable by a person, e.g., visual, audible and/or haptic feedback, indicating or otherwise based on position information determined by an intubation guidance system, method, or device. Visual intubation guidance information may be displayed via a video monitor of a video laryngoscopy system (e.g., a monitor carried by a handheld VL camera or blade, or a monitor connected to the hand-held VL camera or blade by a cable), via visual indicator(s) (e.g., LEDs) integrated with, secured to, or located proximate the handheld VL camera or blade, or via any other suitable visual indicators. In addition or alternatively, intubation guidance information may include audible feedback output by one or more speakers, and/or haptic feedback, e.g., via vibration of the VL or other system component.

Some embodiments provide intubation guidance systems, methods, and devices configured to determine and monitor 2-dimensional or 3-dimensional position information regarding an endotracheal tube relative to a video laryngoscope, and display guidance information indicating or otherwise based on such position information via a display device, e.g., a video monitor, LCD, or LED(s), for example, which display device may be integrated with, secured to, or distinct from the video laryngoscope.

References in this document to detecting or monitoring the “endotracheal tube” or “ET” (e.g., in the preceding paragraph) are intended to refer to any aspect or component of a styletted endotracheal tube, e.g., the stylet, the tube itself, or other structure(s) or component(s) integrated with or secured to a styletted endotracheal tube, except where it is clear from the context of the respective reference to “endotracheal tube” or “ET” is referring only to the tube itself. In addition, it should be understood that any of the concepts and embodiments disclosed herein may be applied to a non-styletted endotracheal tube, e.g., where one or more detectable elements 46 (e.g., magnets or other detectable elements 46) are integrated in or otherwise secured to or associated with a non-styletted endotracheal tube for detection and monitoring of the tube using one or more detection sensors 24 (e.g., magnetometer(s) or other types of sensors) and data analysis and user feedback provided by data analysis system 28.

It should also be understood that any of the concepts and embodiments disclosed herein may be applied to any other instruments (i.e., other than an endotracheal tube) that are inserted in a patient's airway, including, for example, a steerable stylet, a bougie, or a fiberoptic scope. That is, one or more detectable elements 46 may be integrated in or otherwise secured to or associated with a steerable stylet, bougie, or fiberoptic scope for detection and monitoring of such instruments using one or more detection sensors 24 (e.g., magnetometer(s) or other types of sensors) and data analysis and user feedback provided by data analysis system 28.

Some embodiments may determine and display a virtual indication of the location of the trachea (or other anatomic structure(s)), as well as a virtual indication of location and/or orientation of a video laryngoscope and/or an endotracheal tube relative to the trachea. Thus, some embodiments may provide real-time information (e.g., via a display device) indicating both (a) the location and/or orientation of an endotracheal tube relative to a video laryngoscope and (b) the spatial relationship of the video laryngoscope and/or styletted endotracheal tube relative to the trachea or other anatomic structures. This information may help a proceduralist understand key spatial relationships that facilitate endotracheal intubation.

In some embodiments, one or more magnets may be secured to or integrated with an endotracheal tube (ETT) or endotracheal stylet (ETS) (or other medical instrument, as discussed above) in a fixed and known position and/or orientation relative to the ETT or ETS (or other medical instrument). One or more magnetic field sensors configured to detect the magnet(s) associated with the ETT or ETS may be secured to or integrated with a visualization tool (e.g., a video laryngoscope) that provides optical visualization of the ETT or ETS. Each magnetic field sensor may be located in a fixed and known position and/or orientation relative to the video laryngoscope, for example, in a fixed position relative to the lens of the video laryngoscope. A processing unit may analyze data from the magnetic field sensors to non-optically determine position information (e.g., a 3-dimensional location and/or orientation) of the ETT or ETS relative to the video laryngoscope. This may be particularly useful when the ETT or ETS cannot be viewed optically, for example when the ETT or ETS is outside the field of view of the video laryngoscope camera, or when the video laryngoscope camera lens is obscured, e.g., by blood, gastric contents, humidity, etc.

One example embodiment provides a system for facilitating an endotracheal intubation including (a) a video laryngoscope apparatus including a video camera configured to capture video images; and one or more magnetometers configured to generate magnetometer signals based on an interaction with one or more magnetic elements associated with an intubation device; (b) a processor communicatively coupled to the one or more magnetometers and configured to receive the magnetometer signals, and calculate position information regarding the intubation device relative to the video laryngoscope apparatus; and (c) an information output device communicatively coupled to the processor and configured to output information to a user indicating or based on the calculated position information regarding the intubation device.

In one embodiment, the information output device of the intubation device guidance system comprises a visual display device including one or more light-emitting diodes (LEDs) or other visual elements.

In one embodiment, the intubation device comprises a styletted endotracheal tube including a stylet configured to be arranged within a flexible endotracheal tube; and the one or more magnetic elements associated with an intubation device comprise one or more magnetic portions of the stylet or one or more magnets secured to the stylet.

In one embodiment, the processor is configured to determine, based on the magnetometer signals, intubation guidance information including at least one of (a) a spatial location of the intubation device, (b) a proximity of the intubation device relative to the video laryngoscope apparatus, or (b) a safety metric regarding the intubation device, and control the information output device to display or otherwise output the intubation guidance information.

In one embodiment, the processor of the intubation device guidance system is configured to determine, based on the magnetometer signals, that the intubation device has advanced to a reference point, axis, or plane associated with a field of view of the video camera, and in response, output a notification via the information output device indicating that the user can switch attention to a video display configured to display video images captured by the video camera of the video laryngoscope apparatus.

In one embodiment, the system for facilitating an endotracheal intubation further includes a machine vision system configured to receive video images captured by the video camera, the video images corresponding with a field of view of the video camera; analyze the received video images to identify the intubation device in the field of view of the video camera; and in response, output a notification via the information output device.

Another embodiment provides a system for facilitating an endotracheal intubation, including (a) a video laryngoscope apparatus including a video laryngoscope body and a video camera arranged near an end of the video laryngoscope body and configured to capture video images; and (b) an intubation device guidance system. The intubation device guidance system may include (a) one or more magnetometers arranged relative to the video laryngoscope body and configured to generate magnetometer signals based on an interaction with one or more magnetic elements associated with an intubation device; (b) a processor communicatively coupled to the one or more magnetometers and configured to receive the magnetometer signals, and calculate position information regarding the intubation device relative to the video laryngoscope apparatus; and (c) an information output device communicatively coupled to the processor and configured to output information to a user indicating or based on the calculated position information regarding the intubation device.

In some embodiments, the intubation device guidance system is physically distinct from the video laryngoscope apparatus.

In other embodiments, the intubation device guidance system is physically integrated with the video laryngoscope apparatus.

In one embodiment, a portion of the video laryngoscope body is configured to be inserted in a laryngoscope blade, and the one or more magnetometers are provided on an intubation guidance system element configured to be arranged at least partially within the laryngoscope blade.

In one embodiment, the intubation device guidance system element is configured to be secured or arranged on or adjacent an outer surface of the video laryngoscope body.

In one embodiment, the intubation device guidance system element defines a sleeve structure configured to receive a portion of the video laryngoscope body.

In one embodiment, the information output device of the intubation device guidance system comprises a visual display device including one or more light-emitting diodes (LEDs) or other visual elements.

In one embodiment, the video laryngoscope body includes a video laryngoscope handle configured to be gripped by a user's hand, and the information output device of the intubation device guidance system comprises a display device configured to be secured to or arranged on the video laryngoscope handle.

In one embodiment, the intubation device comprises a styletted endotracheal tube including a stylet configured to be arranged within a flexible endotracheal tube, and the one or more magnetic elements associated with an intubation device comprise one or more magnetic portions of the stylet or one or more magnets secured to the stylet.

In some embodiments, the intubation device guidance includes only a single magnetometer. In other embodiments, the intubation device guidance includes multiple magnetometers.

Another embodiment provides an intubation guidance apparatus for facilitating an intubation. The intubation guidance apparatus may include one or more non-optical sensors configured to generate non-optical sensor signals based on interactions with one or more detectable elements associated with an intubation device, and a processor coupled to the one or more non-optical sensors magnetometers and configured to (a) receive the non-optical sensor signals, (b) determine position information regarding the intubation device based on the non-optical sensor signals, (c) generate intubation guidance information based on the determined position information regarding the intubation device, and (d) communicate the intubation guidance information for output to a user via an information output device.

In one embodiment, the intubation device comprises a styletted endotracheal tube including a stylet configured to be arranged within a flexible endotracheal tube, the one or more detectable elements comprise one or more magnetic elements associated with the styletted ETT, and the one or more non-optical sensors comprise one or more magnetometers configured to detect the one or more magnetic elements associated with the styletted endotracheal tube.

Although the present disclosure describes embodiments that include (a) magnet(s) associated with an ETT or ETS and (b) magnetometer(s) associated with a video laryngoscope and configured to detect the magnet(s) associated with an ETT or ETS, the magnets and magnetometers may similarly be provided in the reverse configuration. That is, the disclosed systems, methods, and devices may include (a) magnet(s) associated with a video laryngoscope and (b) magnetometer(s) associated with an ETT or ETS and configured to detect the magnet(s) associated with the video laryngoscope, for determining position information of the ETT or ETS relative to the video laryngoscope (and/or relative to the trachea or other anatomical landmark), or vice versa.

The discussions provided herein focus on detecting and monitoring the location and/or orientation of a stylet, e.g., a magnetized stylet. However, it should be understood that any embodiments disclosed herein may similarly be configured to detect and monitor the location and/or orientation of any other detectable element or structure associated with an endotracheal tube, stylet, or other medical device, including, for example, the endotracheal tube itself (as opposed to the stylet). For example, an endotracheal tube may have one or more magnetized elements or regions integrated in or secured to the endotracheal tube.

Some embodiments may include or operate in cooperation with an external neck sensor apparatus placed on the outside surface of a patient's neck and configured to determine the location of the trachea (or other anatomical features), which may be displayed to the proceduralist via a virtual display to facilitate a non-optical intubation. For example, some embodiments may incorporate or operate in cooperation with any of the neck apparatuses or techniques disclosed in any of the following applications (collectively the “Neck Sensor Applications”):

U.S. provisional patent application Ser. No. 61/993,275 filed May 15, 2014;

U.S. provisional patent application Ser. No. 62/104,682 filed Jan. 16, 2015;

U.S. provisional patent application Ser. No. 62/117,461 filed Feb. 18, 2015;

U.S. patent application Ser. No. 14/714,189 Filed May 15, 2015; and

PCT application PCT/US2016/013954 filed Jan. 19, 2016 (published as WO2016115571A9).

The entire contents of the Neck Sensor Applications are hereby incorporated by reference.

The Neck Sensor Applications disclose, among other things, a neck apparatus that is placed on the outside of a person's neck and may be used, for example, to facilitate an intubation of the person. The neck apparatus may include sensors for locating the trachea or other anatomic landmarks. In addition, the neck apparatus may also include sensors (such as magnetometers) for identifying the location of an endotracheal stylet (ETS). By determining the location of both the trachea and the ETS, the endotracheal tube can be guided into the trachea using a virtual at a computer, smartphone, or other display device.

### BRIEF DESCRIPTION OF THE FIGURES

Example aspects and embodiments of the present invention are described in detail below with reference to the following drawings:

FIG. 1 illustrates an example guided intubation system including a sensor-based stylet guidance system for determining position information of a styletted endotracheal tube relative to a video laryngoscope and providing intubation guidance information to a user based on the determined position information, to facilitate an intubation procedure, according to example embodiments.

FIG. 2A illustrates an example guided intubation system including a sensor-based stylet guidance system integrated in a handheld video laryngoscope (VL) device, according to an example embodiment. FIGS. 2B and 2C illustrate an example camera/sensor apparatus configured to be integrated (e.g., permanently) with a handheld VL device, e.g., in the embodiment of FIG. 2A, according to an example embodiment.

FIG. 3 illustrates a relative arrangement of a styletted ETT and a portion of a handheld VL device, to illustrate various aspects of the present disclosure.

FIGS. 4A-4C illustrate an example removable sensor-based intubation guidance apparatus configured to be arranged adjacent a distal portion of a handheld VL device and arranged in a detachable VL blade, according to one example embodiment.

FIG. 5 illustrates an assembly process for a removable sensor-based intubation guidance apparatus comprising a guidance system sleeve inserted into a disposable VL blade and including a magnetometer array, according to example embodiments.

FIG. 6 illustrates an example method for performing an intubation procedure using a guided intubation system that monitors a stylet position and identifies a camera plane crossing, according to an example embodiment.

FIG. 7 illustrates an example method for an intubation process assisted by a magnet-based guidance system and a machine vision system for identifying a camera plane crossing, according to an example embodiment.

FIG. 8 illustrates another example method for an intubation process assisted by a magnet-based guidance system and a machine vision system for identifying a camera plane crossing, according to another example embodiment.

FIGS. 9A-9C illustrate definitions for “laterality,” “depth,” and “penetration” of a styletted endotracheal tube relative to a video laryngoscope, according to an example embodiment configured to determine these three positional parameters of an endotracheal tube, according to an example embodiment.

FIGS. 10A-10E illustrate an example scheme for indicating the detected “laterality,” “depth,” and “penetration” of the styletted endotracheal tube relative to a video laryngoscope, using an LED display including an array of LEDs, according to an example embodiment.

FIG. 11 illustrates an example triangulation algorithm for determining a location of a styletted endotracheal tube, according to an example embodiment.

FIG. 12 illustrates an example of data-base search for algorithm creation and algorithm clinical use for determining a location of a styletted endotracheal tube, according to an example embodiment.

FIG. 13 illustrates another example algorithm for determining a location of a styletted endotracheal tube, according to an example embodiment.

FIG. 14 illustrates another example algorithm for determining a location of a styletted endotracheal tube, according to an example embodiment.

FIG. 15 illustrates an example hybrid algorithm (e.g., a hybrid of the algorithms shown in FIGS. 12 and 13) for determining a location of a styletted endotracheal tube, according to an example embodiment.

FIGS. 16A-16D illustrate an example embodiment of an intubation guidance system that includes a multi-colored LED display integrated in, attached to, or otherwise located on or at the handle of a video laryngoscope device, for indicating a current state of a guided intubation procedure, according to an example embodiment.

FIG. 17 illustrates an example state-based algorithm for providing guidance-based facilitation of an intubation procedure using a magnet-based stylet guidance system, according to an example embodiment.

FIG. 18 illustrates an example magnetometer calibration algorithm, according to one embodiment.

FIG. 19 illustrates an example magnet detection algorithm, according to one embodiment.

FIG. 20 illustrates an example camera plane crossing detection algorithm, according to one embodiment.

FIG. 21 illustrates another example camera plane crossing detection algorithm, according to another embodiment.

FIG. 22 illustrates an example VL blade including magnetometers arranged in “rings” along a length of the BL blade, according to one embodiment.

FIG. 23 illustrates an example algorithm for calculating a stylet proximity metric, according to one example embodiment.

FIG. 24 illustrates another example algorithm for calculating a stylet proximity metric, according to another example embodiment.

FIG. 25 illustrates an example algorithm for calculating a stylet safety level, based on detected stylet location and orientation (e.g., pitch/yaw), according to an example embodiment.

FIG. 26 illustrates an example algorithm for calculating stylet pitch/yaw data for a stylet including two or more detectable magnets, according to one embodiment.

FIG. 27 illustrates an example algorithm for calculating stylet pitch/yaw data for a stylet including a single detectable magnet, according to one embodiment.

FIG. 28 illustrates an example state-based algorithm for providing guidance-based facilitation of an intubation procedure using a magnet-based stylet guidance system, for a case without magnetometer calibration, according to an example embodiment.

FIG. 29 illustrates a magnet detection algorithm for detecting whether a magnet is nearby, for a case without magnetometer calibration, according to one embodiment.

FIG. 30 illustrates an example neck apparatus that may be used in certain embodiments of the present invention.

FIG. 31 illustrates an example display showing a virtual indication of the position of a patient's trachea with respect to the neck, as well as the position of an endotracheal tube in relation to the trachea, as detected by an example neck apparatus, according to an example embodiment.

FIGS. 32A-32C illustrate an example intubation guidance system including a video laryngoscope and an acoustic or electromagnetic imaging system for detecting and providing a virtual display of relevant components (e.g., endotracheal tube) or anatomical features (e.g., trachea), to, for example, facilitate intubation when the video laryngoscope camera is obstructed, according to an example embodiment.

FIGS. 33A and 33B illustrate an example VL video display for a VL system with and without non-optical sensing, for a situation in which the VL camera is blocked/occluded, according to an example embodiment.

FIG. 34 illustrates an example system for collecting and analyzing signals generated by an array of 3D magnetometers integrated into a video laryngoscope, e.g., to determine the location or orientation of an endotracheal tube relative to the laryngoscope camera, and displaying the determined endotracheal tube location or orientation via a video display, according to an example embodiment.

FIG. 35 illustrates an example clinical algorithm in the setting of intubation when the VL camera is obstructed, but with guidance from the neck sensor apparatus, according to an example embodiment.

FIG. 36 illustrates an example clinical algorithm in the setting of intubation without neck sensor apparatus, where the VL camera is not obstructed, using magnetic guidance system for localizing the ETS with respect to the VL in order to avoid palate or oropharynx trauma, according to an example embodiment.

### DETAILED DESCRIPTION

The present disclosure is generally directed to systems, methods, and devices configured to determine position information (e.g., 3D location and/or orientation) of a medical device (e.g., an endotracheal tube or endotracheal stylet) relative to (a) another medical device (e.g., a video laryngoscopy camera and/or a non-optical imaging device (e.g., a non-optical neck apparatus)), and/or (b) one or more anatomical features (e.g., the trachea, tonsils, vocal cords, etc.). Some example applications include use as an airway assessment device and/or intubation guidance system. Other applications include determination of the 3-D location and/or orientation of a medical instrument relative to a non-optical imaging device, e.g., an infrared camera, or x-ray generator.

Some embodiments are configured to identify the location and/or spatial orientation of a styletted endotracheal tube relative to a video laryngoscope device. Some embodiments include a styletted endotracheal tube including one or more magnets (e.g., one or more magnetized portions of the stylet) and a video laryngoscope including an array of magnetic field sensors (magnetometers) arranged along the longitudinal length of the video laryngoscope and configured to detect the magnet(s) associated with the endotracheal stylet. The system may further include a processor configured to execute suitable algorithms or other computer code to analyze the signals from the magnetometer(s) to determine position information (e.g., position and/or orientation) regarding the stylet and control one or more output device(s) to output guidance information to a user (e.g., intubation proceduralist) based on the determined stylet position information. In some embodiments, the processor may be further configured to determine that the stylet tip has advanced beyond a camera plane and is thus in view of the VL camera (unless the camera is occluded), and output a corresponding notification such that the proceduralist may switch his or her attention to the VL video monitor.

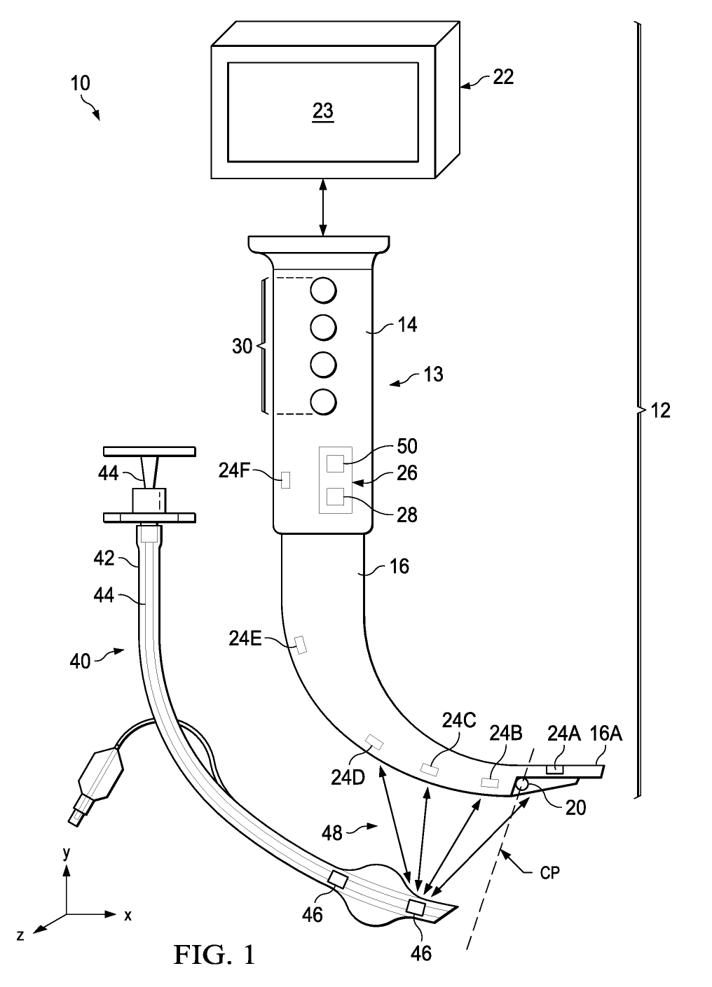

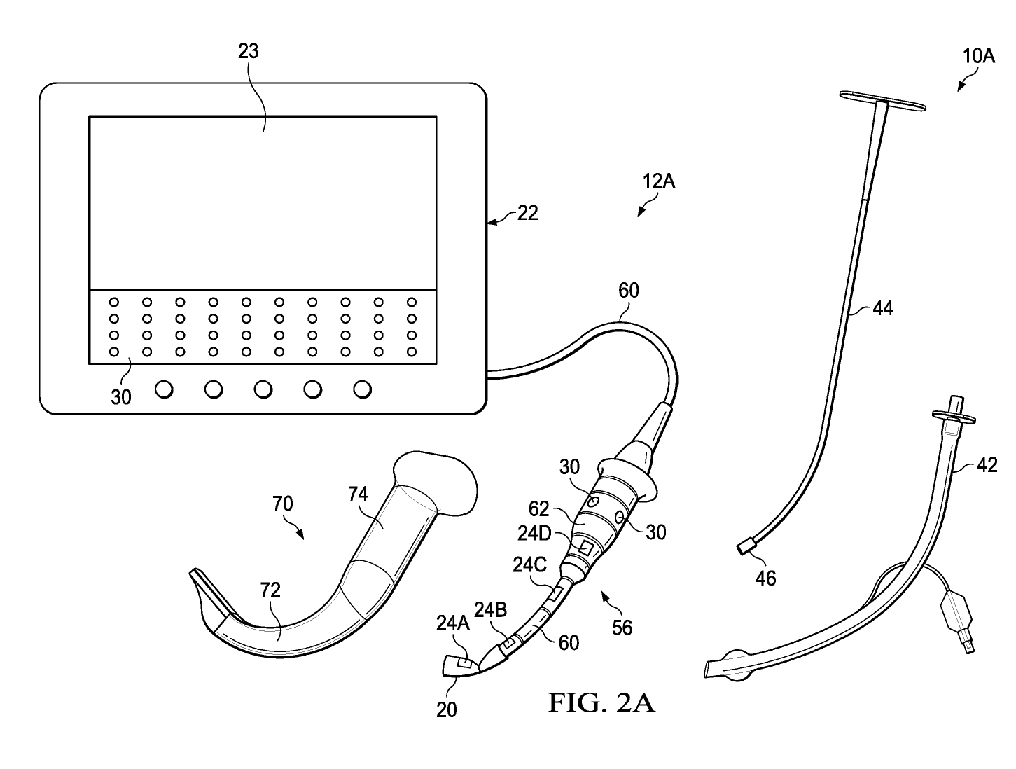

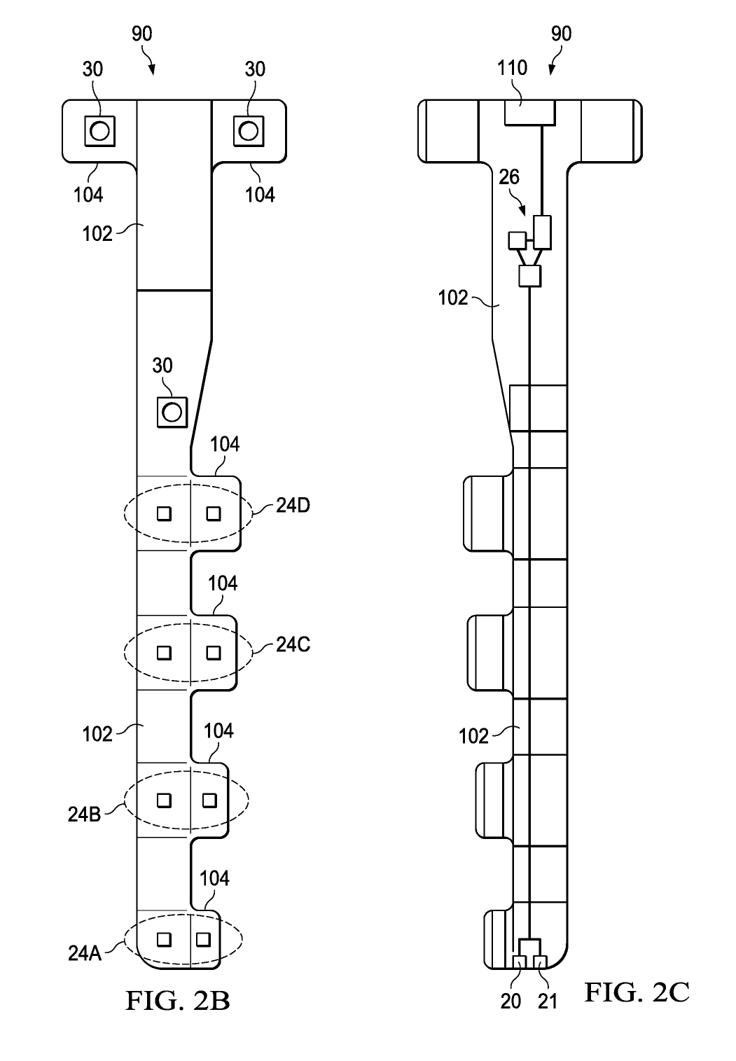

FIG. 1 illustrates an example guided intubation system 10, according to example embodiments. Guided intubation system 10 may include a video laryngoscope (VL) system 12 and an styletted endotracheal tube (ET) 40 including an endotracheal tube 42 having a flexible stylet 44 inserted therein. Video laryngoscope (VL) system 12 may include a handheld device 13 including a handle portion 14 and a blade 16 extending from handle 14, a video camera (or camera lens) 20, and a video monitor 22. Video camera 20 may be connected to video monitor 20 by a fiber optic cable that passes through handle portion 14. In some embodiments, handle portion 14 and blade 16 may be formed as a single integral unit. In other embodiments, blade 16 is detachably connected from handle 14. For example, in some embodiments, e.g., as shown in FIG. 2A, a hollow disposable blade 16 is detachably coupled to handle portion 14, such that camera 20 and distal end of the fiber optic extend through an interior space within blade 16. In some embodiments, blade 16 may include a distal extension portion 16A projecting in an insertion direction beyond camera 20 and configured to lift the epiglottis or vallecula.

VL camera 20 may define a camera plane indicated at “CP” that distinguishes a camera-viewable region in space (downstream of the camera) that is visible to VL camera 20 from a camera-hidden region in space (upstream of the camera) that is not visible to the VL camera. As discussed herein, guided intubation system 10 may utilize this position information to identify camera plane crossing events (e.g., including insertion-direction and/or withdrawal-direction camera plane crossing events) and automatically trigger one or more functions or actions in response to such events, for example, automatically displaying a notification for the proceduralist to switch attention from visual indicator(s) 30 of the intubation guidance system (e.g., one or more colored LEDs) to VL video display 23 on monitor 22, and vice versa.

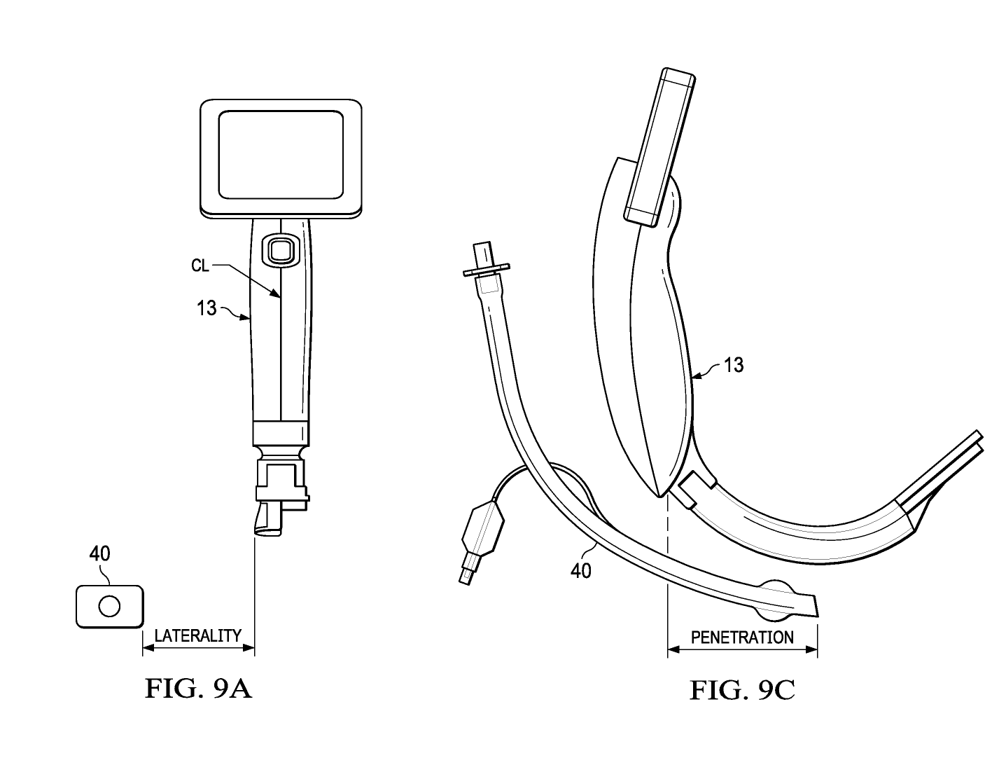

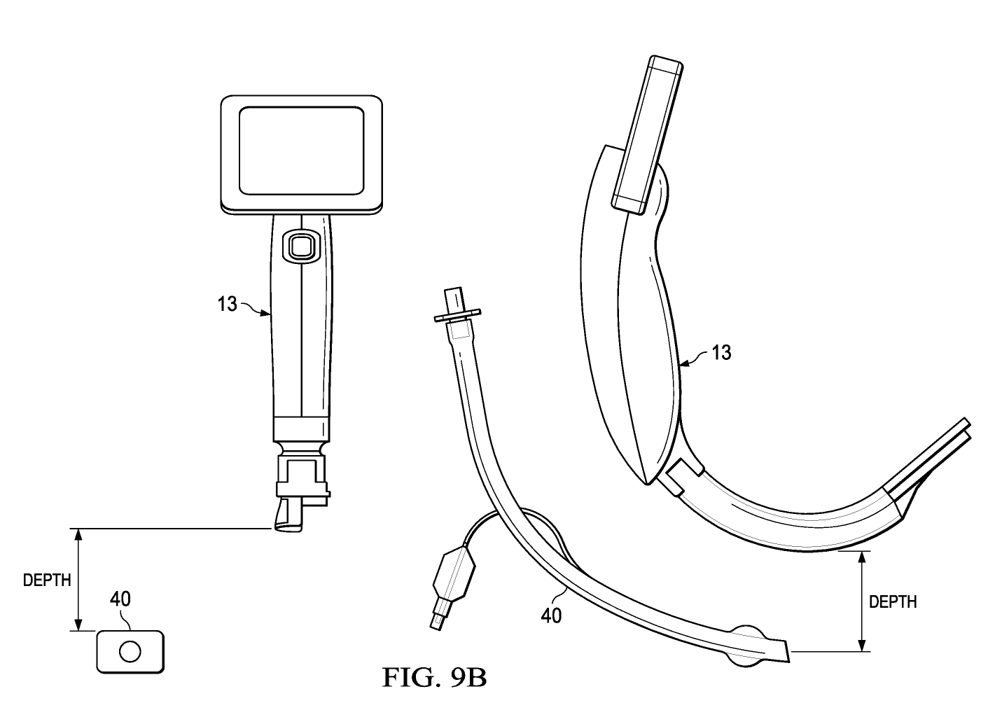

In some embodiments, video monitor 22 may be formed integral with or otherwise physically secured to handheld VL device 13, e.g., as shown in FIGS. 9A-9C. In other embodiments, video monitor 22 may be distinct and/or remote from handheld VL device 13 and connected to handheld VL device 13 via a cable, e.g., including a fiber optic cable remotely connected to camera 20 and/or conductive elements for transferring power and/or data communications to/from handheld VL device 13, e.g., as shown in FIG. 2A. Video monitor may include a video screen 23 configured to display video images captures by camera 20. Further, in some embodiments, video monitor 22 may be configured to display intubation guidance information via video screen 23 or via one or more other visual indicators provided by video monitor 22, as discussed below.

Guided intubation system 10 may include a non-optical sensor-based stylet guidance system 48 for determining position information (e.g., 3-D position and/or orientation) of styletted endotracheal tube 40 relative to handheld VL device 13 and outputting intubation guidance information to a user based on the determined position information, to facilitate an intubation procedure. As shown in FIG. 1, in some embodiments, the non-optical sensor-based stylet guidance system 48 may include:

(a) a single detectable element 46 or multiple detectable elements 46 associated with styletted ETT 40;

(b) a single detection sensor 24 or multiple detection sensors 24 associated with handheld VL device 13 and configured to detect the detectable element(s) 46 associated with styletted ETT 40;

(c) one or more guidance information output devices configured to output guidance information to a proceduralist or other user, e.g., to facilitate an intubation procedure; and

(d) electronics 26 configured to analyze signals from detection sensor(s) 24, determine position information regarding detectable element(s) 46 (and by extension, position information regarding styletted ETT 40), and control guidance information output device(s) to provide guidance information indicating or based on the determined position information.

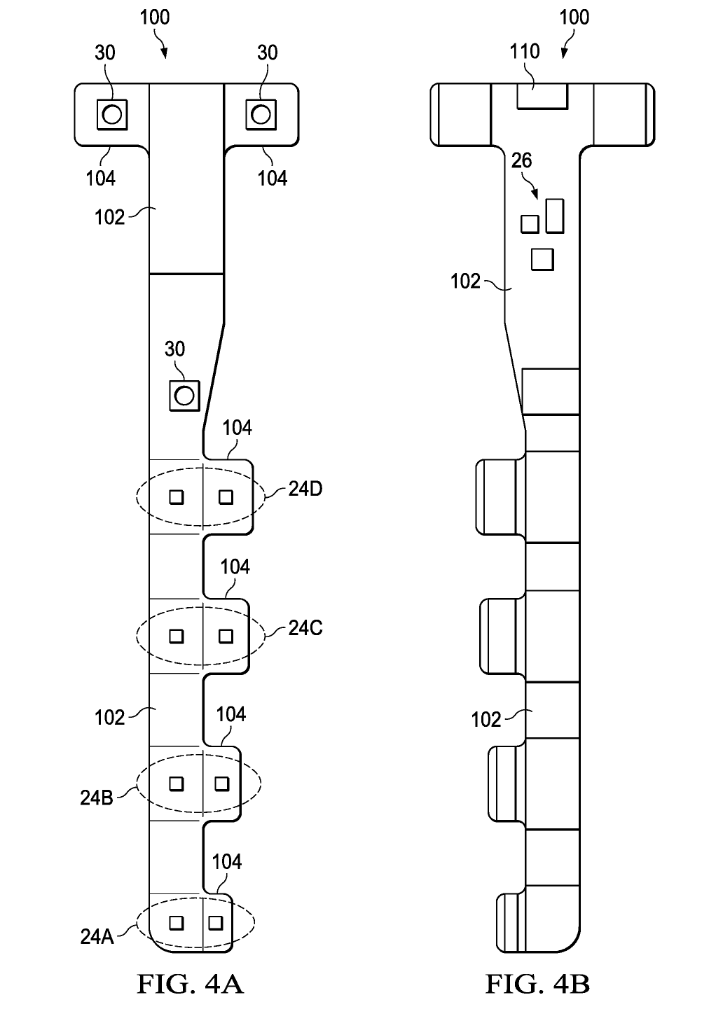

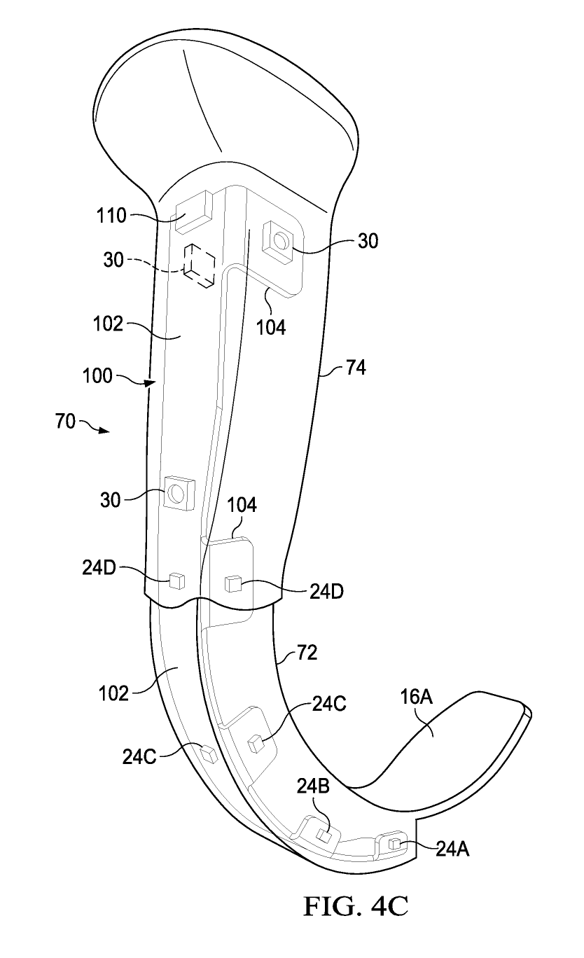

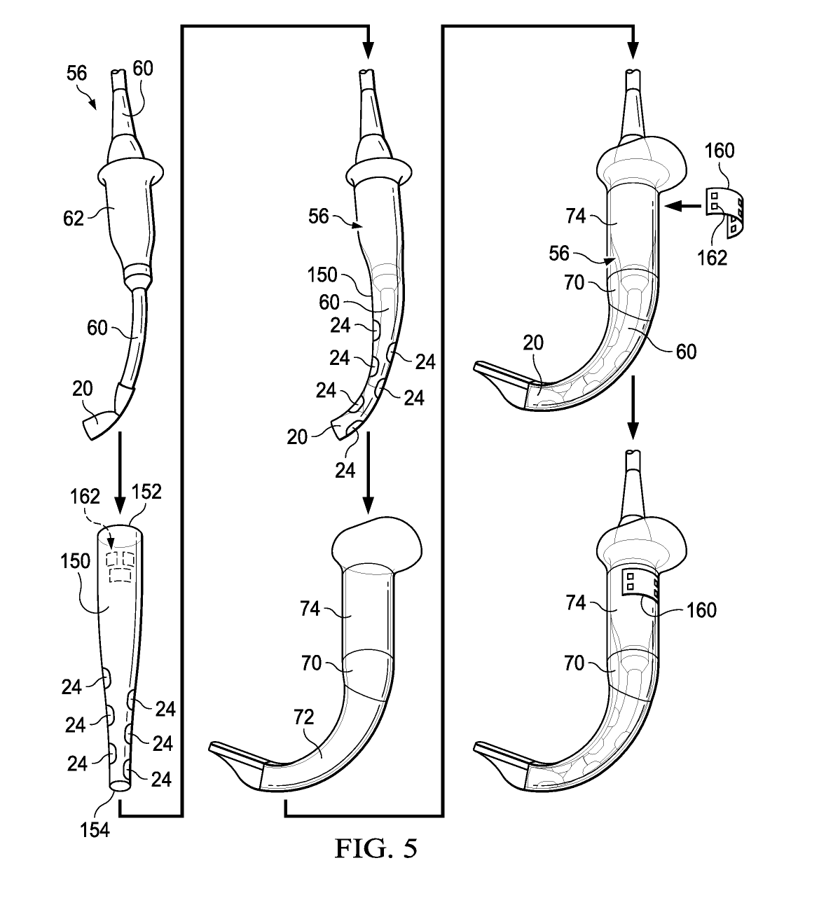

In some embodiments, some or all elements of non-optical sensor-based stylet guidance system 48 are manufactured integrally with handheld VL device 13 or other component(s) of V system 12. Thus, guided intubation system 10 may comprise a self-contained video laryngoscopy system with an integrated sensor-based (e.g., magnet-based) stylet guidance system 48. In other embodiments, e.g., as shown in FIGS. 4 and 5, some or all components of sensor-based stylet guidance system 48 may be separate from a respective VL system but configured to cooperate with the VL system, e.g., as an after-market complementary system, to provide sensor-based monitoring and feedback to facilitate an intubation procedure

A detectable element 46 may include any element that is detectable by a suitable detection sensor 24, and a detection sensor 24 may include any sensor or device that is configured to detect a detectable element 46. In some embodiments, detectable elements 46 may comprise one or more magnetized regions of an ETT stylet 44, and detection sensor(s) 24 may comprise a single magnetometer 24 or multiple magnetometers 24 configured to detect each magnetized region of ETT stylet 44 (i.e., each detectable element 46). In other embodiments, detectable elements 46 may include RF emitter (e.g., RFID), ultrasonic/acoustic emitter or reflector, infrared emitter, visible-light-based emitters, etc., and detection sensors 24 may include RF detector, ultrasound sensors, thermal cameras, photodetectors, etc. Further, in some embodiments, detection sensors 24 and detectable elements 46 may be arranged in a reverse manner as shown in FIG. 1, i.e., detectable elements 46 may be included in or otherwise associated with VL device 13 and detection sensors 24 may be included in or otherwise associated with ETT 40.

In some embodiments, the intubation guidance system is configured for use with a proprietary magnetized stylet, e.g., by analysis of the interaction between magnetometers secured to the VL device (e.g., at the handle and/or fiber optic cable) and the magnetized stylet. The magnetized stylet may include any number and orientation of magnets or magnetized regions. As examples only, the magnetized stylet may include a single local magnet at the distal end of the stylet, or may include multiple local magnets arranged spaced-apart along the length of the stylet, or may include one or more elongated magnets/magnetized regions extending along the length of the stylet, or the entire length of the stylet may be magnetized.

In one embodiment, the intubation guidance system is configured to detect the VL system being powered on, e.g., using a using voltage or current regulator in the guidance system sleeve to detect current flowing through the VL system, and automatically wake the intubation guidance system from a sleep/lower-power mode and activate the intubation guidance system display (e.g., LEDs). Thus, the user only needs to power the VL system on/off to also automatically power the intubation guidance system on/off.

Intubation Stylet

The magnetic intubation stylet 44 may provide an external magnetic field that can be detected by the magnetometers (e.g., 3D magnetometers) associated with the video laryngoscope. All or any portion(s) of stylet 44 may be magnetized. In some embodiments, the video laryngoscope with an embedded array of magnetometers is calibrated to work with a magnetic stylet with a particular size, shape, and magnetic field profile. In some embodiments, the stylet has several magnets incorporated that are oriented in different directions. For example, two magnets can be arranged orthogonally to each other, such that their magnetic fields produce a greater sphere of detection. Stated differently, by having magnets oriented in different directions, the distance at which the magnetometers can detect the magnetic field is increased along the sphere of detection (i.e. the radius of detection is increased).

In one embodiment, the magnetic intubation stylet is flexible and conformable, such that it can assume the shape desired by the operator. Some operators may wish to bend the stylet near its distal end or proximal end. When multiple magnets are present along the length of the ETT and the position and orientation of each magnet is known, a curve can be created through the magnets. The curvature will be representative of flexion. Additionally, electronics may be added to the ETT in order to create a resistive-based sensor, or other sensor known to one skilled in the art, that measures flexion. The stylet may be made be thin enough such that it can be reversibly inserted inside a standard endotracheal tube.

The magnet can be attached to the ETT through soldering the magnetic tip to the non-magnetic portion of the stylet. The magnet could also be adhered using adhesive or an epoxy overmold. Alternatively, a plastic or metal micro-enclosure could orient and affix the magnet to the end stylet. This enclosure could permanently or reversibly clamp onto the end of the stylet. The magnet can be incorporated into any portion of the stylet, or the entire stylet could be magnetized.

In one embodiment, the distal end of the intubation stylet is magnetic. For example, the distal end of the stylet can be composed of a neodymium cylinder magnet that is ⅛″ in diameter and 1″ long. In another embodiment, there are two magnets arranged along the length of the stylet: one at the distal tip of the stylet and another more proximally. In another embodiment, the entire stylet is magnetic. In this embodiment, the stylet may be composed of a weaker magnetic material such as iron, or nickel, or other material known to one skilled in the art. This approach may be easier to manufacture as the stylet would be a single material. While location of the tip specifically could not be determined, the location of the whole stylet would be determined through detection of the relative magnetic fields sensed by multiple magnetometers.

Those familiar with the art will recognize that there are many possible ways of incorporating a magnet into the intubation stylet, or magnetizing all or a portion of the intubation stylet. As mentioned previously, any device or apparatus that is intended to be inserted into or near the trachea (such as a bougie, stylet, endotracheal tube, suction catheter, nasal airway, laryngoscope blade tip, etc.) can be magnetized in a fashion that will enable guidance using the techniques described herein.

Guidance Information Output Devices

Guidance information output devices for outputting guidance information to a proceduralist or other user may include any suitable devices for outputting visual, audible, haptic, or other type of human-perceptible information to a user. For example, the guidance system may include one or more guidance indicators 30 (e.g., an LCD or LED screen or one or more discrete LEDs) configured to display guidance information indicating or based on position information of styletted ETT 40 determined by electronics 26. In some embodiments, such guidance indicator(s) 30 may be integral with, secured to, or otherwise associated with handheld VL device 13 (e.g., one or more LEDs integrated in or secured to VL handle portion 14 as shown in FIG. 1), or may be integral with, secured to, or otherwise associated with VL monitor 22.

In some embodiments, e.g., as discussed below with reference to FIGS. 31-34, electronics 26 may display certain guidance information via video screen 23 of video monitor 22. For example, electronics 26 may display virtual information regarding the location of VL device 13, stylet 40, and/or anatomical features of the patient (e.g., the trachea) in combination with video images captured by camera 20.

Guidance information may indicate, for example (a) a detected position and/or angular orientation of styletted ETT 40, (b) whether the ETT 40 is positioned/oriented properly or improperly during an intubation procedure, (c) camera plane crossing events (e.g., indicating the distal tip 46 of stylet 44 has crossed camera plane CP and has thus become visible/hidden via video screen 23), and/or any other information for assisting an intubation procedure.

In some embodiments, guidance information may be displayed via the video laryngoscope monitor (which may be integrated with the video laryngoscope device or provided as a separate unit (e.g., on a wheeled cart), a computer display (e.g., a display of a desktop computer, laptop computer, or tablet computer), a smartphone display, or any other type of display device. In such embodiments, the visual display device may display stylet position information or other guidance information based on the stylet position information. In addition, the depth, location, boundary limits, or other parameters of the patient's trachea or other anatomic landmarks can be visually represented on the relevant video display, e.g., a smart phone, VL monitor, computer screen, etc. Information regarding relative location of devices or structures can be displayed via an overlay of the video laryngoscope feed, a separate 3D-simulated model, or through multiple 2D displays that show the relative location in distinct planes, for example.

In some embodiments, guidance information output devices may include audio and/or haptic feedback devices for outputting position information. For example, system 10 may include a speaker configured to output defined tones or voice-based feedback or instructions based on detected position information of ETT 40, to provide any of the various types of guidance information discussed above. Such speaker may be located in the VL handle 14, monitor 22, or other component of system 10. As another example, system 10 may include vibration device(s) configured to output haptic feedback to the user based on detected position information of ETT 40. System 10 may control one or more vibration parameters, e.g., vibration magnitude, vibration pulse duration, pattern of multiple vibration pulses, etc., to communicate corresponding position information. For example, system 10 may adjust the magnitude of vibration pulses as a function of the extent to which ETT 40 is misaligned or misoriented with respect to a proper alignment or orientation. As another example, system 10 may generate a defined pattern of vibration pulses, e.g., 3 short pulses, to indicate an insertion-direction camera crossing event, which informs the proceduralist to switch attention to the VL camera view displayed via screen 23.

Acoustic user interfaces can include chimes to indicate successful or unsuccessful milestones in the procedures. This can include a positive chime for successful placement or a warning chime if the endotracheal tube is either in the esophagus or too far from the video laryngoscope or in jeopardy of contacting an airway structure (i.e. tonsil). A speaker could also emit varying frequencies to communicate a parameter such as tube depth or distance of the tube from the video laryngoscope. For example, as the tube travels further from the video laryngoscope, the audio frequency could increase which would alert the user of this non-ideal behavior.

Haptic user interfaces may employ off-balance motors to create vibration feedback. A haptic user interface may be included in secure to the VL handle. An advantage of this technique is that information may be communicated to the user un-obstructively and without the creation of potentially distracting noise and can be used in a loud environment. Haptic feedback may communicate information similar to acoustic feedback. Known vibration patterns may act as chimes, and continuous vibration frequencies could communicate information for a given parameter.

Electronics and Data Analysis System

Electronics 26 may include a data analysis system 28 including one or more processors, memory devices, and computer-readable instructions stored in the memory device(s) and executable by the processor(s) to perform any of the guidance related functions disclosed herein. The computer instructions may include any suitable algorithms or other computer code embodied as software, firmware, or in any other suitable manner for performing any of the functions disclosed herein. For example, data analysis system 28 may include software or firmware executable to analyze signals from detection sensor(s) 24, determine position information regarding detectable element(s) 46 associated with styletted ETT 40, and control guidance information output device(s) to provide guidance information to a user indicating or based on the determined position information.

Data analysis system 28 may analyze signals from magnetometer(s) 24 to determine and monitor position information regarding stylet 44 (and thus, styletted ETT 40) relative to VL device 13 and/or relative to detected anatomical landmark(s) of the patient (e.g., the trachea), and control one or more guidance information output devices (e.g., guidance indicator LEDs 30, a speaker, or a haptic feedback device) to output intubation guidance information indicating or otherwise based on the determined stylet position information, to facilitate an intubation procedure. In some embodiments, data analysis system 28 may analyze signals from magnetometer(s) 24 to determine any or all of the following categories of stylet position information:

Stylet spatial location information indicating a spatial location of stylet 44 or each magnetic region of stylet 44 in one, two, or three dimensions (e.g., along the x, y, and/or z axes) relative to VL device 13 or specified point(s) associated with VL device 13, e.g., relative to camera lens 20, relative to a distal tip of VL blade 16, relative to one or more magnetometer(s) 24, or relative to any other specified point or points within or on a surface of VL device 13.

Stylet distance information indicating a distance between stylet 44 or each magnetic region of stylet 44 and VL device 13 or specified point(s) associated with VL device 13 in one, two, or three dimensions (e.g., along the x, y, and/or z axes).

Stylet orientation information indicating an angular or rotational orientation of stylet 44 or a potion of stylet 44 (e.g., proximate magnetized region(s) 46 of stylet 44) relative to one or more specified axes, e.g., the x, y, and/or z axes. In some embodiments, stylet orientation information may indicate a pitch, yaw, and/or roll of stylet 44.

Stylet penetration information indicating how far the magnetized stylet 44 has been advanced along the longitudinal axis of the VL device 13. The stylet penetration metric may be calculated based on any of the spatial location information, distance information, and/or angular/rotational orientation information discussed above.

Camera plane crossing information indicating whether a specified portion or point on stylet 44 (e.g., corresponding with a magnetized region 46 at the distal tip of stylet 44) has crossed a “camera plane” defined by camera 20 in an insertion (downstream) and/or withdrawn (upstream) direction.

Data analysis system 28 may analyze signals from magnetometer(s) 24 and/or any stylet position information determined from such magnetometer signals and output (e.g., via indicator(s) 30 and/or video monitor 22) intubation guidance information based on such analysis. In some embodiments, data analysis system 28 may determine and monitor state information (including state changes) regarding stylet 44, e.g., by comparing magnetometer signals and/or stylet position information to one or more threshold values maintained by data analysis system 28 or otherwise analyzing such data. For example, data analysis system 28 may determine any of the following state information based on magnetometer signals and/or determined stylet position information:

Stylet proximity state (e.g., close, medium, far, etc.) may indicate the distance of stylet 44 from VL device 13 and may be determined based on (a) determined stylet distance information (e.g., by comparing stylet distance information in one or more dimensions with respective threshold values for each defined state (e.g., close, medium, or far), and/or other input data), (b) stylet orientation information (e.g., by comparing a determined pitch, yaw, and/or roll of stylet 44 with respective threshold values), and/or (c) other input data.

Camera plane crossing state (e.g., stylet upstream of camera plane, camera plane crossed in an insertion (downstream) direction, stylet downstream of camera plane, camera plane crossed in a withdrawal (upstream) direction) may be determined based on (a) analysis of raw magnetometer signals (e.g., comparing magnetometer signal magnitudes with respective threshold values for each plane crossing state), (b) a determined stylet proximity state (e.g., close, medium, far, etc.), (c) stylet orientation information and/or (d) other input data.

Guidance system state (e.g., off, standby mode, active mode, insertion complete, etc.) may be determined, e.g., based on (a) analysis of raw magnetometer signals (e.g., comparing magnetometer signal magnitudes with respective threshold values for each defined guidance system state), (b) a determined stylet proximity state (e.g., close, medium, far, etc.), (c) stylet orientation information, (d) a determined camera plane crossing state (e.g., stylet upstream of camera plane, camera plane crossed in an insertion (downstream) direction, stylet downstream of camera plane, camera plane crossed in a withdrawal (upstream) direction), and/or (e) other input data.

Intubation safety state (e.g., safe, warning, danger) may indicate the current level of safety/danger during an intubation procedure, and may be determined, e.g., based on based on (a) analysis of raw magnetometer signals (e.g., comparing magnetometer signal magnitudes with respective threshold values for each defined guidance system state), (b) a determined stylet proximity state (e.g., close, medium, far, etc.), (c) stylet orientation information, (d) a determined camera plane crossing state (e.g., stylet upstream of camera plane, camera plane crossed in an insertion (downstream) direction, stylet downstream of camera plane, camera plane crossed in a withdrawal (upstream) direction), and/or (e) other input data.

Data analysis system 28 may generate and output any suitable intubation guidance information based on any of the stylet position information, state information, an/or any other suitable information accessible to or determined by data analysis system 28. As discussed above, data analysis system 28 may output intubation guidance information via any suitable guidance system output device, e.g., via guidance indicator(s) 30 and/or via video monitor 22. For example, data analysis system 28 may indicate any current state or state change via guidance indicator(s) 30 and/or video monitor 22.

Although electronics 26 are shown in FIG. 1 as located in VL handle 14, electronics 26 may be located in any one or more components of system 10. For example, electronics 26 may include a processor, memory, and software/firmware provided in monitor 22, and a secondary processor (e.g., provided on a microcontroller) and other electronics (e.g., a multiplexer, etc.) provided in VL handle 14 and communicatively coupled to the electronics in monitor 22.

Camera Plane Crossing

As discussed above, data analysis system 28 may determine camera plane crossing events and/or camera plane crossing status information indicating, e.g., whether a specified portion or point on stylet 44 (e.g., a magnetized element at/near the distal end of stylet 44) has crossed a “camera plane” that distinguishes a region in space viewable by VL camera 20 (e.g., downstream of camera 20) from a region in space not viewable by VL camera 20 (e.g., upstream of camera 20). Upon detecting an insertion direction camera plane crossing, data analysis system 28 may output a notification (e.g., a visual, audible, or haptic notification) informing the proceduralist that it is safe or appropriate to switch look away from the mouth and toward the VL video screen 23.

As discussed above, data analysis system 28 may determine camera plane crossing events/status using magnetic tracking, that is, using magnetometer(s) 24 associated with VL device 13 to detect magnetic elements 46 associated with styletted ETT 40. In some embodiments, system 10 may also include a “machine vision” system 50 configured to analyze video images captured by VL camera 20 to identify the presence/absence of an ETT and/or ETT stylet within the field of view of camera 20. In some embodiments, machine vision system 50 may be configured to detect one or more identifiable features of or associated with ETT 42 and/or stylet 44, e.g., one or more machine-identifiable or “machine-readable” colors, shapes, markings, or patterns associated with ETT 42 and/or stylet 44, portions of ETT 42 and/or stylet 44, or elements formed integral with or otherwise secured to ETT 42 and/or stylet 44. Machine vision system 50 may use a processor to execute any known or other suitable algorithms (e.g., embodied in software or firmware of system 10) to identify the presence or absence of an ETT and/or ETT stylet from video images captured by VL camera 20.

Machine vision system 50, acting alone, may have limited effectiveness when camera 20 is blocked or occluded, for example, by blood or other substance in the airway or on the camera lens, or by condensation or moisture on the camera lens.

Thus, some embodiments of system 10 include both (a) magnetic tracking of stylet 44 (via detection of magnetic element(s) 46 by magnetometer(s) 24) and (b) machine vision system 50 as inputs for determining camera plane crossing events or camera plane crossing status, which may improve the camera plane crossing analysis, by providing redundancy and increased accuracy. In some embodiments, data analysis system 28 may use the magnetic tracking data and data from machine vision system 50 (e.g., the presence/absence of ETT 42 or stylet 44 in the camera view) as discrete, redundant inputs. For example, data analysis system 28 may (a) determine whether the magnetic tracking system detects a camera plane crossing and (b) determine whether machine vision system 50 detects the presence of ETT 42 or stylet 44, and (c) identify a camera plane crossing upon a positive determination by at least one of the magnetic tracking system and the machine vision system 50 (or alternatively, only upon a positive determination by both of the magnetic tracking system and the machine vision system 50).

In some embodiments, data analysis system 28 may analyze the magnetic tracking data and data from machine vision system 50 (e.g., the presence/absence of ETT 42 or stylet 44 in the camera view) collectively to identify a camera plane crossing event. For example, data analysis system 28 may (a) determine a “magnetic tracking confidence metric” representing a statistical confidence that the magnetic tracking data indicates a camera plane crossing, (b) determine a “machine vision confidence metric” representing a statistical confidence of a visual detection of ETT 42 or stylet 44 by machine vision system 50, (c) mathematically combine the two confidence metric to compute a combined plane crossing confidence metric, and (d) compare the combined plane crossing confidence metric with a defined threshold value to identify a camera plane crossing event, and if so, output a notification to the proceduralist as discussed above.

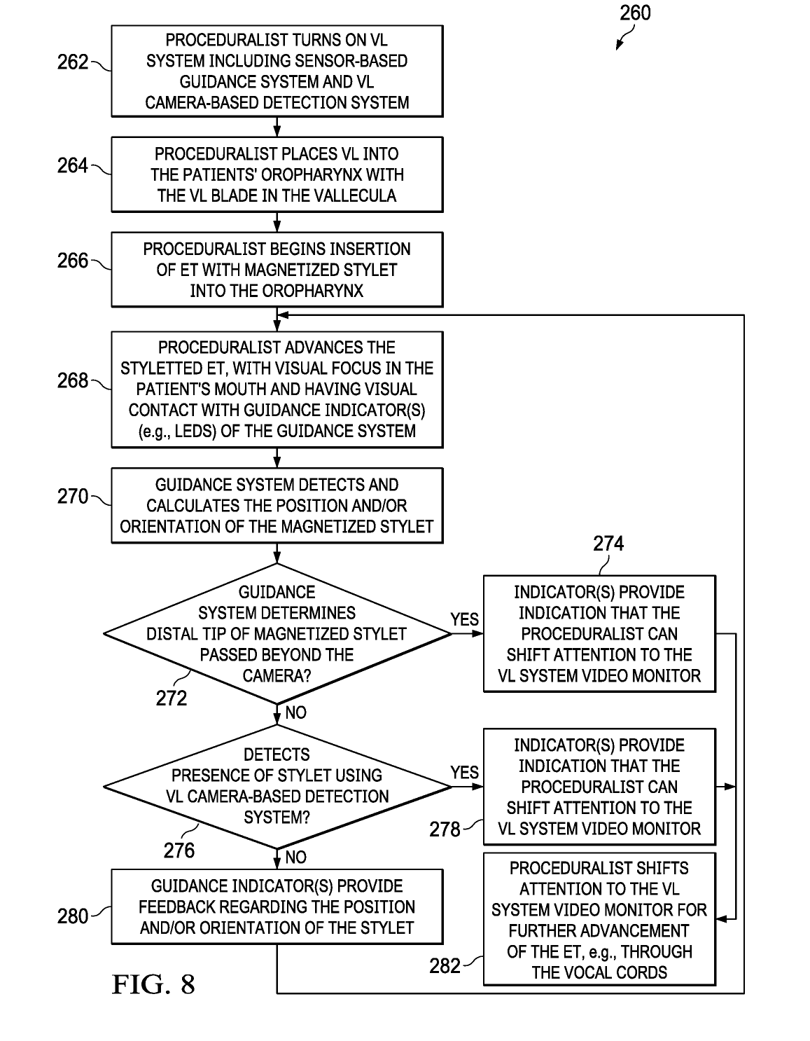

Example methods for using both magnetic tracking and machine vision data for identifying camera crossing events during an intubation process are shown in FIGS. 8 and 9, which are discussed below. At least in some situations, using a magnetic tracking system as disclosed herein in combination with a machine vision system 50 for identifying camera plane crossing events/status may provide increased accuracy and reliability as compared with using either system alone.

Integrated Guided Intubation System

FIG. 2A illustrates an example guided intubation system 10A including a video laryngoscope (VL) system 12A, an endotracheal tube (ET) 42, and an endotracheal stylet 44 including one or more magnetized regions 46, according to an example embodiment. VL system 12A may include a camera 20 connected to a video monitor 22 by an optical cable 60, a VL housing 62 formed around the optical cable 60, and a hollow disposable blade 70 configured to receive a distal portion of optical cable 60 terminating at video camera 20. The portion of VL system 12A extending from (and including) VL housing 62 to camera 22 may define a handheld VL device 56 configured to be received within disposable blade 70. In particular, disposable blade 70 may include a lower blade portion 72 that receives camera 20 and an upper handle portion 74 that receives VL housing 62. VL housing 62 may house or include any components discussed above with respect to system 10 shown in FIG. 1. For example, VL housing 62 may house or include one or more detection sensors (e.g., magnetometers 24), any electronics 26 discussed above (e.g., data analysis system 28, machine vision system 50, etc.), guidance indicator(s) 30 (e.g., one or more LEDs), and/or any other system components.

System 10A may include a non-optical sensor-based stylet guidance system 48, which may be manufactured integrally with VL system 12A, including an array of detection sensors 24, e.g., magnetometers or other sensors, integrated with or secured to handheld VL device 56, e.g., integrated with or secured to optical cable 60, VL housing 62, and/or camera 20. For example, detection sensors 24 may be arranged along the longitudinal length of handheld VL device 56, e.g., extending from VL housing 62 to the distal end of optical cable 60, i.e., at or proximate camera 20. Detection sensors 24 may any type of sensor(s) configured to detect one or more detectable elements 46 integrated with or secured to ETT 42 or stylet 44.

In some embodiments, handheld VL device 56 may include an array of magnetometers 24 configured to detect one or more magnetized portions 46 of stylet 44. Data analysis system 28 may be configured to analyze signals from magnetometer(s) 24 to determine and monitor stylet position information of stylet 44 (inserted in ETT 40) relative to the magnetometers 24 included in or secured to handheld VL device 56, and control one or more guidance information output devices (e.g., video screen 23 or guidance indicators 30 provided in video monitor 22, LEDs or other guidance indicators 30 included in or secured to handheld VL device 56, a speaker, or a haptic feedback device) to output intubation guidance information indicating or otherwise based on the determined stylet position information, to facilitate an intubation procedure, as discussed herein.

In some embodiments of guided intubation system 10A, magnet-based stylet guidance system 48 may be permanently integrated with VL system 12A. Some embodiments include a combined camera/sensor apparatus 100, e.g., carried by a common PCB or other substrate, that provides the VL video camera 20 along with the components of magnet-based stylet guidance system 48.

For example, FIGS. 2B and 2C illustrate an example camera/sensor apparatus 90 configured to be integrated (e.g., permanently) with handheld VL device 56, e.g., within a flexible or semirigid outer housing of handheld VL device 56, e.g., as represented by cable 60 or housing 62 shown in FIG. 2A, or any other suitable housing. FIGS. 2B and 2C show a front side and rear side, respectively, of the camera/sensor apparatus 90, showing three LEDs 30 and four pairs of magnetometers 24 mounted on an elongated flexible PCB 102. As shown in FIG. 2B, two LEDs 30 and one magnetometer 24 in each pair is mounted on a flange or “wing” 104 extending laterally from the elongated PCB 102. As shown in FIG. 2C, VL video camera 20 and an illumination LED 21 may be mounted to PCB 102. Various electronics 26 may also be mounted on PCB 102, including, for example, any or all of the following: a multiplexer (MUX) configured to multiplex and pass on the signals generated by magnetometers 24A-24D; data analysis system 28, video processing electronics configured to process signals from VL camera 20, which may include a machine vision system 50 configured to analyze signals from VL camera 20 to identify the presence/absence of an ETT or stylet; and/or any other suitable electronics.

Camera/sensor apparatus 90 may also include a connection interface 110 for a wired connection to one or more other system components, e.g., a component that includes larger electronics of the intubation guidance system (e.g., a battery, processor(s), etc.), or a separate video monitor or display device (e.g., including one or more LEDs), such as a display device configured to be secured to the VL handle or a display device of an existing VL system, e.g., an LCD video screen attached to or integrated with the VL handle or remote from the VL handle, for example. In one embodiment, connection interface 110 may be configured for a wired connection to a secondary guidance system component.

When installed within a VL housing (e.g., during manufacturing), PCB flanges or wings 104 may flex or fold relative to the main elongated body of the flexible PCB 102, to fit within VL housing. As a result of the flexing/folding of the flanges or wings 104, each pair of magnetometers 24 are orientated in different planes, which may provide additional magnetometer data for determining position information regarding a magnetized stylet 44.

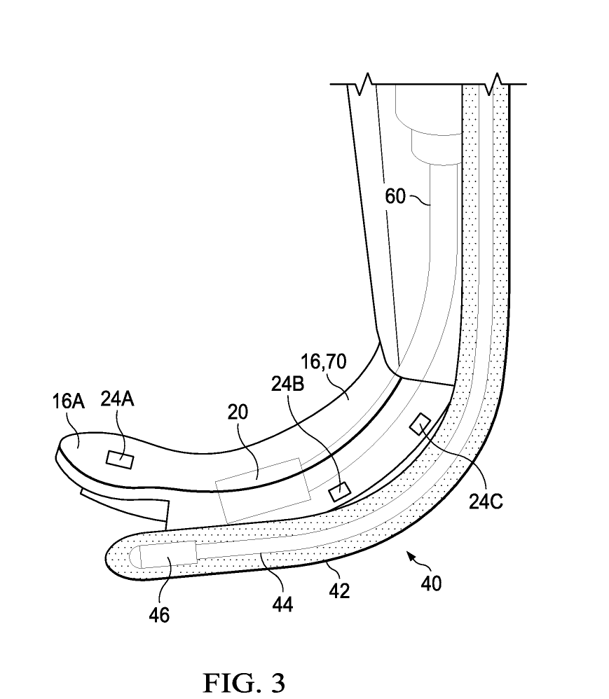

FIG. 3 illustrates a relative arrangement of a styletted ETT 40 and a portion of a handheld VL device, to illustrate various aspects of the present disclosure. The arrangement shown in FIG. 3 may correspond with system 10 shown in FIG. 1 or system 10A shown in FIG. 2. Thus, the handheld VL device may comprise, for example, a disposable blade 70 housing a camera 20, optical cable 60, and relevant electronics, or an integrated, reusable VL blade 16, e.g., including a camera 20, optical cable 60, and relevant electronics housed in a titanium blade housing. As shown, a plurality of detections sensors 24 (e.g., magnetometers) may be arranged along the length of the handheld VL device, which may be configured to detect one or more detectable elements 46 associated with the styletted ETT 40 (e.g., magnetized portion(s) of stylet 44 or magnet(s) secured to stylet 44 or ETT 42).

Removable Sensor-Based Guidance Apparatus

In some embodiments, a guided intubation system may include a removable sensor apparatus configured to be removably arranged inside a detachable VL blade, e.g., blade 70 shown in FIG. 2. The removable sensor apparatus may include an array of detection sensors 24 (e.g., magnetometers), one or more guidance indicators 30 and/or other electronics 26 provided on a carrier, e.g., a flexible printed circuit board (PCB) or sleeve structure.

The removable sensor apparatus may be arranged adjacent a handheld VL device (e.g., camera and optical cable) received in a detachable VL blade. For example, an alternative embodiment of FIG. 2A may replace the detection sensors 24 (e.g., magnetometers) integrated with handheld VL device 56 with detection sensors 24 provided on removable sensor apparatus that can be inserted inside blade 70 prior to insertion of handheld VL device 56, such that the sensor apparatus is arranged between handheld VL device 56 and the inner surface of blade 70.

FIGS. 4A-4C illustrate an example sensor apparatus 100 configured to be removably arranged inside a detachable or disposable VL blade 70 adjacent a handheld VL device (e.g., camera, optical cable, and handle portion) also inserted in the VL blade 70, according to one example embodiment. Sensor apparatus 100 may be generally similar to camera/sensor apparatus 90 shown in FIGS. 2B-2C and discussed above, but may exclude the VL camera 20 and illumination LED 21, as such components may be provided by the handheld VL device inserted adjacent sensor apparatus 100 in the detachable or disposable VL blade 70.

FIGS. 4A and 4B show a front side and rear side, respectively, of the removable sensor apparatus 100, showing three LEDs 30 and four pairs of magnetometers 24 mounted on an elongated flexible PCB 102. As shown in FIG. 4A, two LEDs 30 and one magnetometer 24 in each pair is mounted on a flange or “wing” 104 extending laterally from the elongated PCB 102. As shown in FIG. 4B, various electronics 26 may also be mounted on PCB 102. Electronics may include a multiplexer (MUX) to multiplex and pass on the signals generated by the various magnetometers 24.

Sensor apparatus 100 may also include a connection interface 110 for a wired connection to one or more other system components, e.g., a component that includes larger electronics of the intubation guidance system (e.g., a battery, processor(s), etc.), or a separate video monitor or display device (e.g., including one or more LEDs), such as a display device configured to be secured to the VL handle or a display device of an existing VL system, e.g., an LCD video screen attached to or integrated with the VL handle or remote from the VL handle, for example. In one embodiment, connection interface 110 may be configured for a wired connection to a secondary guidance system component (e.g., configured to clip onto the VL handle or fiber optic cable), e.g., similar to clip 160 shown in FIG. 5 or a similar component.

FIG. 4C shows example sensor apparatus 100 inserted in an example VL blade 70. In this embodiment, the PCB flanges or wings 104 on which certain LEDs 40 and magnetometers 24 are mounted are configured to flex or fold relative to the main elongated body of the flexible PCB 102, to fit within VL blade 70. As a result of the flexing/folding of the flanges or wings 104, each pair of magnetometers 24 are orientated in different planes, which may provide additional magnetometer data for determining position information regarding a magnetized stylet 44. A handheld VL device, e.g., similar to handheld VL device 56 shown in FIG. 2, may be inserted in VL blade 70, e.g., after insertion of sensor apparatus 100, such that sensor apparatus 100 lies adjacent to the handheld VL device.

In one embodiment, removable sensor apparatus 100 may define a complete one-piece intubation guidance system, e.g., including the components shown in FIGS. 4A-4C, a power source (e.g., battery), microprocessor(s), memory device(s), and/or any other components for providing any or all of functionality disclosed herein.

FIG. 5 illustrates an example embodiment of a part removable intubation guidance system, according to one example embodiment. In one embodiment, removable intubation guidance system is a two-part system includes (a) a guidance system sleeve 150 including one or more detection sensors 24 (e.g., 3D magnetometers) designed to be arranged over a portion of a handheld VL device and then inside a disposable video laryngoscopy blade, and (b) a secondary guidance system component 160 including additional electronic components 162 (e.g., LEDs, a battery, processer, etc.) that may be connected to the electronics of the guidance system sleeve 150 by a cable or other interface. The two-part removable intubation guidance system may define a self-contained system for detecting, monitoring, and providing user feedback regarding the position of an ETT stylet. Guidance system sleeve 150 may include a number of detection sensors 24 (e.g., 3D magnetometers) configured to detect one or more detectable elements 46 associated with a styletted ETT, e.g., a magnetized stylet 44 including one or more discrete single-point or elongated magnets arranged along the length of the stylet 44. Guidance system sleeve 150 may be rigid or flexible.

Secondary guidance system component 160 may be configured to include or carry larger electronic components 162 (e.g., one or more guidance indicators (e.g., LEDs) 30, a battery, a microprocessor and/or microcontroller, user interface button(s), etc.). Thus, secondary guidance system component 160 may provide power, user-input (i.e. on/off switch), data analysis capabilities, and/or guidance feedback to a user. Secondary guidance system component 160 may be rigid or semi-rigid, and may be designed to clip onto an outside surface of blade handle portion 74 of disposable blade 70 (e.g., in the example embodiment shown in FIG. 5). Secondary guidance system component 160 may be connected to guidance system sleeve 150 by any suitable connection interface, e.g., a cable or wireless interface.

As shown in FIG. 5, a handheld VL device 56 may be inserted into through an opening 152 in a first end of guidance system sleeve 150 such that camera 20 is arranged at an opening 154 at a second end of sleeve 150. The handheld VL device 56 and guidance system sleeve 150 may be inserted into a disposable blade 70. Secondary guidance system component 160 may then be removably attached to the handle portion 74 of VL blade 70, e.g., by clipping or snapping onto the VL blade 70 or directly to handheld VL device 56 or optical cable 60.

In other embodiments, secondary guidance system component 160 may be designed to clip onto handheld VL device 56 (e.g., onto VL housing 62 or optical cable 60) prior to insertion of handheld VL device 56 and sleeve 150/secondary guidance system component 160 into disposable blade 70. In still other embodiments, secondary guidance system component 160 may be designed to clip onto handheld VL device 56 (e.g., onto VL housing 62 or optical cable 60) at a location that is clear of (e.g., upstream of) the installed disposable blade 70, and thus may be clipped into place before or after insertion of handheld VL device 56 and sleeve 150 into disposable blade 70.

In other embodiments, the removable intubation guidance system is embodied as a single-piece sleeve 150 including detection sensors 24 (e.g., 3D magnetometers) as well as additional electronic components 162 (e.g., LEDs, a battery, processer, etc.). Such an embodiment is indicated in the lower left portion of FIG. 5 by the optional electronics 162 (indicated by dashed lines) integrated into sleeve 150. Such embodiment may eliminate the separate secondary guidance system component 160 and thus provide a single-piece, self-contained system for detecting, monitoring, and providing user feedback regarding the position of an ETT stylet.

In one embodiment, guidance system sleeve 150 may be stored in a rolled-up configuration designed to be rolled onto the VL device 56 like a condom, starting at the camera end and then rolling upwards towards and/or over the VL handle. Sleeve 150 may include flexible electronics including an array of 3D magnetometers 24. As noted above, the distal end of sleeve 150 may have an opening 154 (or may be clear) so that sleeve 150 does not obstruct the camera view of the VL camera 20. Sleeve 150 may be designed to fit snugly around VL device 56, such that the disposable blade 70 can fit over the VL device 56 and sleeve 150.

In alternative embodiments, guidance system sleeve 150 may be designed as an elongated strip or any other structure configured to be secured in any suitable manner between the disposable VL blade 70 and the fiber optic cable 60 and/or VL housing 62. For example, guidance system sleeve 150 may be designed as an elongated adhesive strip configured to be adhered to an outer surface(s) of fiber optic cable 60 and/or VL housing 62, or to the inside of disposable blade 70, wherein such elongated adhesive strip may include multiple magnetometers 24 arranged along the length of the adhesive strip. In yet another embodiment, disposable blade 70 may be manufactured with integrated magnetometers 24 arranged along the longitudinal length of the blade.

Guided Intubation with VL Camera Plane Detection

As discussed above, a guided intubation system (e.g., including any of the systems or apparatuses shown in FIGS. 1-5 or discussed above) may comprises a sensor-based stylet guidance system configured to determine and monitor position information regarding a styletted ETT and output guidance information indicating or based on such position information via any suitable guidance information output device, to thereby facilitate an intubation procedure. In some embodiments the sensor-based stylet guidance system may be used with a video laryngoscope to facilitate a VL-based intubation process, e.g., to help guide the insertion of a styletted ETT when the ETT not viewable by the VL camera, such as when the ETT has not been advanced into the camera's view and/or when the VL camera is blocked or unreliable.

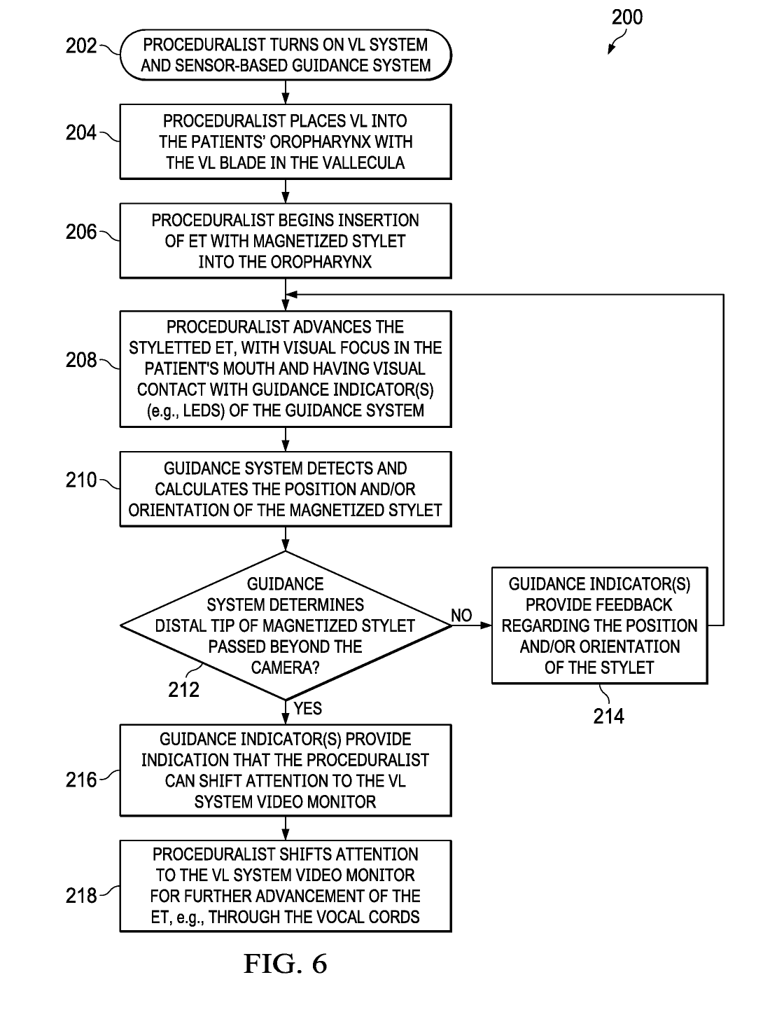

FIGS. 6-8 illustrate example methods for performing an intubation using a guided intubation system including a video laryngoscope system 12 with a non-optical sensor-based stylet guidance system 48, according to example embodiments. In the examples shown in FIGS. 6-8 and discussed below, the non-optical sensor-based stylet guidance system 48 comprises a magnet-based stylet guidance system 48 that includes magnetometer(s) 24 provided in or at the handheld video laryngoscope device that are configured to detect magnet(s) or magnetized region(s) of or associated with the stylet 44, e.g., as discussed above. However, guidance system 48 may utilize any other types of detection sensors 24 and detectable elements 46. Further, the stylet guidance system 48 may be integrated with or separate from video laryngoscope system 12.

FIG. 6 illustrates an example method 200 for performing an intubation procedure using a guided intubation system that includes a video laryngoscope system 12 and (or including) a magnet-based stylet guidance system 48 for detecting and monitoring a styletted endotracheal tube 40, according to example embodiments.

At 202, a proceduralist turns on the video laryngoscope system 12 and magnet-based stylet guidance system 48. As noted above, the stylet guidance system 48 may be integrated with or separate from video laryngoscope system 12. Where the stylet guidance system 48 is integrated with the video laryngoscope system 12, the full system may be turned on by a single interface, e.g., switch or button. Where the stylet guidance system 48 is distinct from the video laryngoscope system 120, the proceduralist may need to turn on each system via a respective interface, e.g., switch or button.

At 204, the proceduralist may hold insert the video laryngoscope 13 into the patient's oropharynx with the VL blade arranged in the vallecula, as known in the art. At 206, the proceduralist may begin insertion of a styletted endotracheal tube (ET) 40 into the oropharynx, while looking into the patient's mouth. The stylet 44 may include one or more magnets or magnetized regions 46 detectable by magnetometers(s) 24 located in, secured to, or otherwise associated with the video laryngoscope 13. At 208, the proceduralist may advance the styletted ETT 40, with visual focus in the patient's mouth and also maintaining visual contact with visual guidance indicator(s) 30 of the stylet guidance system 48, e.g., LEDs or other visual indicator(s) on or near the VL handle.

At 210, as the styletted ETT 40 is advanced relative to the video laryngoscope 13, data analysis system 28 of stylet guidance system 48 may detect and calculate position information regarding the magnetized stylet 44 by analyzing signals from magnetometer(s) 24. At 212, data analysis system 28 of stylet guidance system 48 may determine whether stylet 44 (or more particularly a defined point of stylet 44, e.g., the distal tip of stylet 44 or the location of a magnetized element 46 on stylet 44) has crossed a camera plane defined by the VL video camera 20. If data analysis system 28 determines that the stylet 44 has not crossed the camera plane, as indicated at 214, data analysis system 28 controls guidance indicator(s) 30, e.g., one or more LEDs, regarding the calculated position information of stylet 44, and the method returns to 208 for continued monitoring by stylet guidance system 48 while the proceduralist continues to advance the styletted ETT 40.

Once data analysis system 28 determines at 212 that the stylet 44 has crossed the camera plane, data analysis system 28 may control guidance indicator(s) 30 (e.g., LEDs provided on or near the VL handle) to output an indication that the proceduralist can shift their attention from the patient's mouth to the VL video monitor 23, as indicated at 216. In response to this visual notification, the proceduralist may shift their attention from the patient's mouth to the VL video monitor 23 at 218, and continue the intubation, e.g., including advancing the ETT though the vocal cords.

In other embodiments, stylet guidance system 48 may provide feedback to the proceduralist via audible or haptic feedback, instead of (or in addition to) visual feedback via guidance indicator(s) 30. In such embodiments, method 200 would be modified accordingly, to effectively facilitate the intubation procedure.

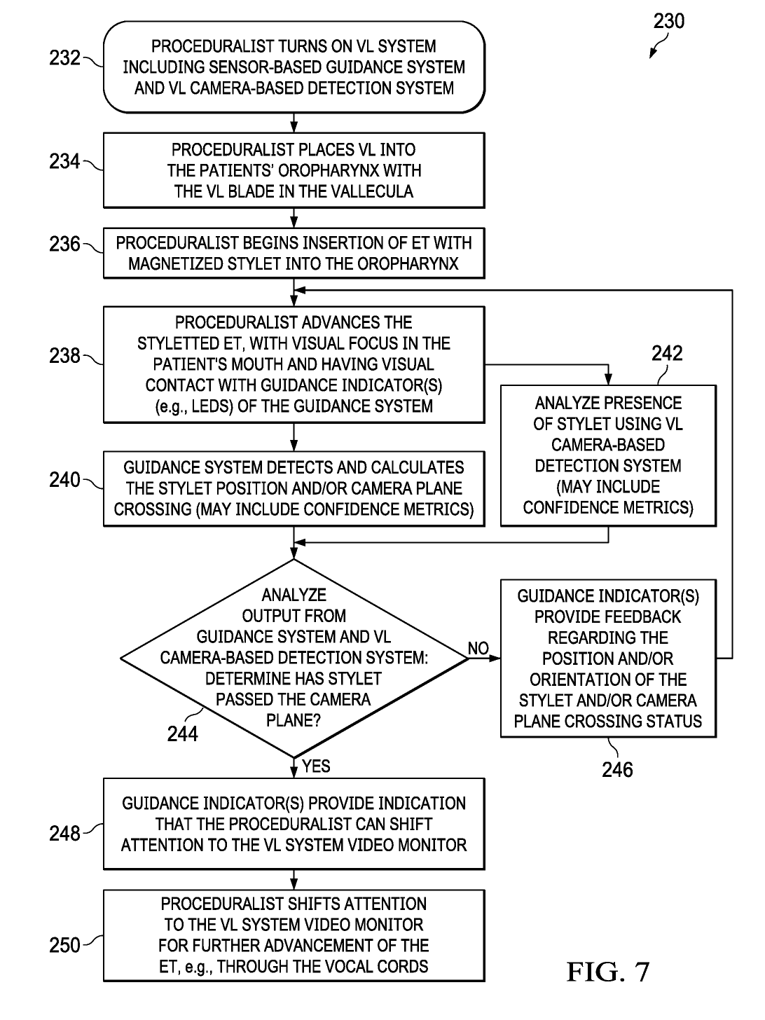

FIG. 7 illustrates an example method 230 for performing an intubation procedure using a guided intubation system that includes a video laryngoscope system 12 with (a) a magnet-based stylet guidance system 48 and (b) a VL camera-based detection system 50 for detecting and monitoring a styletted endotracheal tube 40, according to an example embodiment. Magnet-based stylet guidance system 48 and/or VL camera-based detection system 50 may be integrated with or separate from VL system 12, depending on the particular embodiment.

Steps 232-238 are similar to steps 202-208 of method 200 of FIG. 6 discussed above. At 232, a proceduralist turns on the video laryngoscope system 12, magnet-based stylet guidance system 48, and VL camera-based detection system 50, using one or more use interfaces. At 234, the proceduralist may hold insert the video laryngoscope 13 into the patient's oropharynx with the VL blade arranged in the vallecula, as known in the art. At 236, the proceduralist may begin insertion of a styletted endotracheal tube (ET) 40 into the oropharynx, while looking into the patient's mouth. At 238, the proceduralist may advance the styletted ETT 40, with visual focus in the patient's mouth and also maintaining visual contact with visual guidance indicator(s) 30 of the stylet guidance system 48, e.g., LEDs or other visual indicator(s) on or near the VL handle.

As the styletted ETT 40 is advanced relative to the video laryngoscope 13, both (a) magnet-based stylet guidance system 48 and (b) VL camera-based detection system 50 may simultaneously (or otherwise in parallel) detect or attempt to detect the styletted ETT 40 at steps 242 and 244. At 240, magnet-based stylet guidance system 48 may analyze magnetometer data to detect magnetized stylet 44 and (a) calculate position information regarding stylet 44 and (b) determine whether stylet 44 has crossed the VL camera plane (and is thus within the camera's view). In some embodiments, magnet-based stylet guidance system 48 may determine confidence metrics regarding whether stylet 44 has crossed the VL camera plane (and is thus within the camera's view).

Simultaneously (or otherwise in parallel), at 242, VL camera-based detection system 50 may analyze video images capture by camera 20 to identify or attempt to identify styletted ETT 40. For example, VL camera-based detection system 50 may execute any suitable algorithms to identify/attempt to identify machine-readable/machine-identifiable markings, colors, shapes, patterns, etc. on stylet 44 or ETT 42, to identify the presence or absence of styletted ETT 40 in the camera view. In some embodiments, VL camera-based detection system 50 may determine confidence metrics regarding the presence or absence of styletted ETT 40 in the camera view.

At 244, the guided intubation system may analyze output from magnet-based stylet guidance system 48 (calculated at 240) and output from VL camera-based detection system 50 (calculated at 242) to determine whether stylet 44 (or styletted ETT 40) has crossed the VL camera plane, and is thus visible via the VL video display 23. For example, system may execute an algorithm to mathematically combine the respective stylet/ETT camera plane confidence metrics determined by magnet-based stylet guidance system 48 and VL camera-based detection system 50, and compare the combined value with a threshold value to determine whether a camera plane crossing event has occurred. As another embodiment, the system may execute an algorithm to compare the respective camera plane crossing confidence metrics calculated by magnet-based stylet guidance system 48 and VL camera-based detection system 50 with respective threshold values (or a common threshold value), and determine whether a camera plane crossing event has occurred based on the results of such comparisons.

If the system determines at 244 that a camera plane crossing event has not occurred, magnet-based stylet guidance system 48 may provide feedback regarding the calculated position information of stylet 44 via guidance indicator(s) 30, e.g., one or more LEDs, at 246, and the method returns to 238 for continued monitoring by stylet guidance system 48 while the proceduralist continues to advance the styletted ETT 40.

Alternatively, if the system determines at 244 that a camera plane crossing event has occurred, the system may provide feedback, e.g., via guidance indicator(s) 30 (e.g., LEDs provided on or near the VL handle or other output device perceptible by proceduralist), indicating that the proceduralist can shift their attention from the patient's mouth to the VL video monitor 23, as indicated at 248. In response to this visual notification, the proceduralist may shift their attention from the patient's mouth to the VL video monitor 23 at 250, and continue the intubation, e.g., including advancing the ETT 40 though the vocal cords.

FIG. 8 illustrates an example method 260 for performing an intubation procedure using a guided intubation system that includes a video laryngoscope system 12 with (a) a magnet-based stylet guidance system 48 and (b) a VL camera-based detection system 50 for detecting and monitoring a styletted endotracheal tube 40, according to another example embodiment. Method 260 is generally similar to method 230 shown in FIG. 7, but specifies that each of (a) magnet-based stylet guidance system 48 and (b) VL camera-based detection system 50 performs an independent camera plane crossing determination, and the system notifies the proceduralist to shift their attention to the VL video monitor 23 upon a positive camera plane crossing detection by either system 48 or 50.

Steps 262-268 are similar to steps 202-208 of method 200 of FIG. 6 and steps 232-238 of method 230 of FIG. 7, discussed above. Advancing to step 270, as the styletted ETT 40 is advanced relative to the video laryngoscope 13, stylet guidance system 48 may detect can calculate position information regarding the magnetized stylet 44 by analyzing signals from magnetometer(s) 24. At 272, stylet guidance system 48 may determine whether stylet 44 has crossed the VL camera plane.

If stylet guidance system 48 determines at 272 that the stylet 44 has crossed the camera plane, stylet guidance system 48 may control guidance indicator(s) 30 (e.g., LEDs provided on or near the VL handle) to output an indication that the proceduralist can shift their attention from the patient's mouth to the VL video monitor 23, as indicated at 274, and the proceduralist may thus shift their attention to the VL video monitor 23 at 282, and continue the intubation, e.g., including advancing the styletted ETT 40 though the vocal cords. Alternatively, if stylet guidance system 48 determines at 272 that the stylet 44 has not crossed the camera plane, the method proceeds to 276, where camera-based detection system 50 determines whether stylet 44 (or styletted ETT 40) has crossed the VL camera plane, and is thus visible via the VL video display 23.

If camera-based detection system 50 determines at 276 that the stylet 44 (or ETT 42) has crossed the camera plane, camera-based detection system 50 may control guidance indicator(s) 30 or other visual indicator(s) to output an indication that the proceduralist can shift their attention from the patient's mouth to the VL video monitor 23, as indicated at 278, and the proceduralist may thus shift their attention to the VL video monitor 23 at 282, and continue the intubation, e.g., including advancing the ETT though the vocal cords. Alternatively, if camera-based detection system 50 determines at 276 that the stylet 44 (or ETT 42) has not crossed the camera plane, the magnet-based stylet guidance system 48 may provide feedback regarding the calculated position information of stylet 44 (calculated at 270) via guidance indicator(s) 30, at 280, and the method returns to 268 for continued monitoring by stylet guidance system 48 while the proceduralist continues to advance the styletted ETT.

Intubation Guidance Systems Configured to Monitor and Display Stylet Position Information (e.g., Stylet Laterality, Depth, and Penetration) and/or Guidance System Status Information

Some embodiments provide an intubation guidance system that displays ETT guidance information at the video laryngoscope handle or otherwise in view of an intubation proceduralist, e.g., while the proceduralist is looking in the patient's mouth. For example, such system may include one or more display devices (e.g., an LCD or series of LEDs) integrated in or at the video laryngoscope handle, which may indicate the position of a styletted endotracheal tube (e.g., having a magnetized stylet) relative to a reference point, axis, or plane associated with the VL device, referred to herein as a “Spatial Reference Element” or “SRE.”

A “Spatial Reference Element” or “SRE” may include, for example, any of the following:

(a) a reference point along the VL blade;

(b) a reference point at the distal tip of the VL blade or a reference point at or adjacent the VL camera;

(c) a linear reference axis extending through the VL device in any direction (e.g., in or relative to the x, y, or z direction), for example, a longitudinal axis extending in the longitudinal direction of the handheld VL device/blade, or a lateral axis extending laterally through a point in or on the surface of the handheld VL device/blade;

(d) a non-linear reference axis, for example, a curved longitudinal axis extending through each of a plurality of magnetometers 24 arranged along a longitudinal length of the handheld VL device/blade (wherein such curved axis may comprise a segment curve defined by a sequence of linear segments between adjacent magnetometers, or a mathematically smoothed representation of such segmented curve);

(e) a longitudinal reference plane extending through a longitudinal axis of the VL device/blade, e.g., extending through centerline CL shown in FIG. 9A; and/or

(f) a transverse reference plane extending orthogonal to a longitudinal center plane, for example, a transverse reference plane passing through a point on or proximate a lens of VL video camera 20, referred to herein as a “VL camera plane” or simply a “camera plane,” e.g., VL camera plane “CP” shown in FIG. 1. The VL camera plane may thus distinguish a 3D space downstream of the VL camera from a 3D space upstream of the VL camera. As discussed herein, some embodiments are configured to detect when a styletted ETT crosses through a VL camera plane in an insertion (downstream) or withdrawal (upstream) direction (a “camera plane crossing event”), and output an appropriate user notification such that an intubation proceduralist may switch their visual focus, e.g., to/from the patient's mouth or to/from a VL video screen 23) in response to the camera plane crossing event.

Some embodiments may provide guidance data for facilitating the intubation procedure, e.g., regarding stylet position information relative to an SRE. In some embodiments, the intubation guidance system may be configured to determine any one or more of (a) a “lateral” position of the ETT relative to an SRE, (b) a “depth” of the ETT relative to an SRE, (c) a measure of “penetration” of the ETT relative to an SRE, (d) a measure of the rotational orientation of the ETT (about the longitudinal axis of the tube) relative to an SRE, or (e) any other measures of the position or orientation of the ETT relative to an SRE.

FIGS. 9A-9C illustrate definitions for “laterality,” “depth,” and “penetration” of the styletted endotracheal tube 40 (e.g., as defined by the location of a magnet 46 or other reference point on stylet 44 or reference axis associated with stylet 44) relative to one or more Spatial Reference Elements (SREs) associated with a VL device/blade to determine such positional information regarding ETT 40.

With reference to FIG. 9A, “laterality” is defined as the distance between the magnetized stylet 44 and a laterality SRE defined by a longitudinal reference plane extending through a longitudinal axis of the VL device/blade, e.g., extending through centerline CL shown in FIG. 9A, or an offset plane parallel to such longitudinal reference plane (e.g., tangential to or passing through a point on a lateral outer surface of the VL device/blade).

With reference to FIG. 9B, “depth” is defined as the distance between the magnetized stylet 44 and a depth SRE defined by a reference point, a linear reference axis, a non-linear reference axis (e.g., curved longitudinal axis extending through a plurality of magnetometers 24 arranged along a longitudinal length of the handheld VL device/blade), or other reference point, axis, or plane.

With reference to FIG. 9C, “penetration” represents an extent to which the magnetized stylet is inserted along the VL device/blade, e.g., in the insertion (downstream) or withdrawal (upstream) direction. Penetration may be measured by the location and/or orientation of magnetized stylet 44 and a penetration SRE defined by any suitable reference point, axis, or plane, e.g., a transverse reference plane extending orthogonal to a longitudinal axis of the VL device/blade, and passing through a defined point along the length of the VL device/blade. In one embodiment, the penetration metric increases as the stylet approaches the camera lens 20, and reaches a maximum value at the point of an insertion direction camera plane crossing, at which the stylet appears in the visual field of the camera.

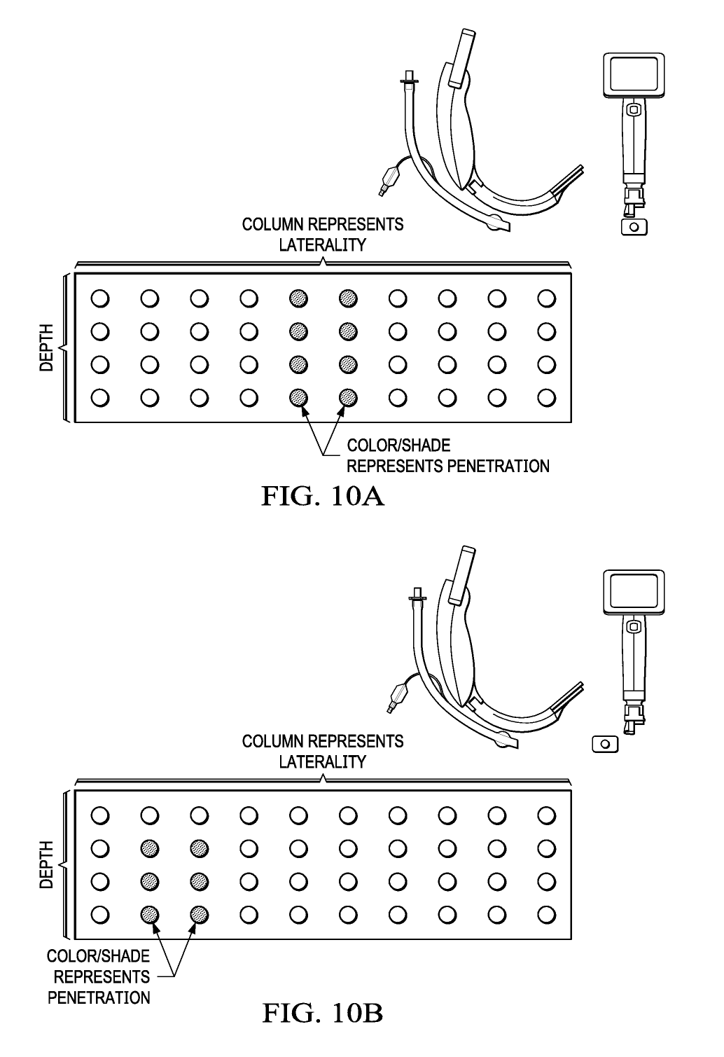

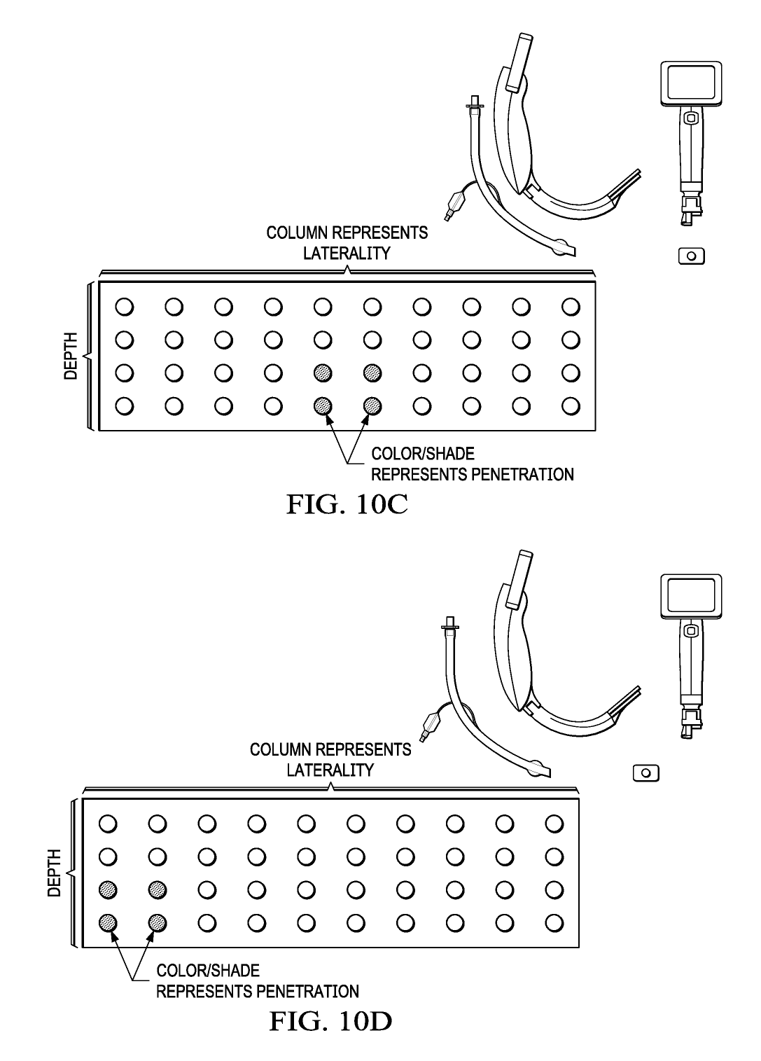

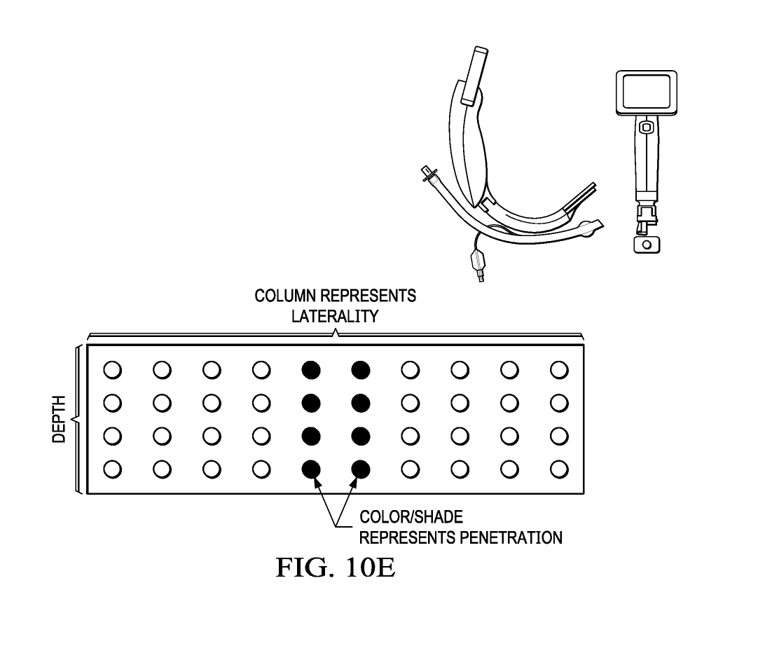

FIGS. 10A-10E illustrate an example user interface and guidance information feedback system for displaying the “laterality,” “depth,” and “penetration” of the styletted ETT 40 relative to the respective laterality SRE, depth SRE, and penetration SRE via an LED array 30, according to one embodiment. The LED array 30 may be integrated in the VL device handle; integrated into the VL screen 23 or monitor 22, e.g., in embodiments in which the video monitor 22 is integrated with or located at the VL handle, e.g., as shown in FIGS. 10A-10E; otherwise integrated into the VL device; or provided as a separate display device, for example. The LED array 30 may be located to be within the field of the proceduralist while the proceduralist is looking in the patient's mouth.

The example LED array 30 includes a two-dimensional array of LEDs, which may be single-colored or multi-colored LEDs. As shown in FIGS. 10A-10E, the 2D LED array 30 may indicate the detected stylet “laterality” by illuminating one or more LED columns laterally corresponding with the detected lateral position of stylet 44. Thus, as data analysis system 28 detects a lateral movement of stylet 44 relative to the laterality SRE associated with the VL device, data analysis system 28 may adjust in real-time the presently illuminated column of LEDs.

Further, as shown in FIGS. 10A-10E, the 2D LED array 30 may indicate the detected stylet “depth” by controlling the number of LEDs illuminated in the particular column(s) presently being illuminated (i.e., the column(s) corresponding with the currently detected stylet laterality, as discussed above), starting with the lowest LED in each respective column and moving upward as a function of decreasing depth (i.e., as the stylet moves closer to the VL device). Thus, as shown in FIG. 10A, all four LEDs in the selected columns are illuminated when stylet 44 is immediately adjacent the VL device; whereas in FIG. 10C, only two of four LEDs in the selected columns are illuminated when stylet 44 is positioned further away from the VL device.

Finally, as shown in FIGS. 10A-10E, the 2D LED array 30 may indicate the detected stylet penetration by controlling the color, shade, and/or brightness of the presently illuminated LEDs as a function of the currently determined penetration metric for stylet 44. For example, comparing FIGS. 10A and 10E, the illuminated LEDs are illuminated with a first color, shade, or brightness level corresponding with a first penetration extent of the stylet (FIG. 10A) and a first color, shade, or brightness level corresponding with the second (greater) penetration extent of the stylet (FIG. 10E).

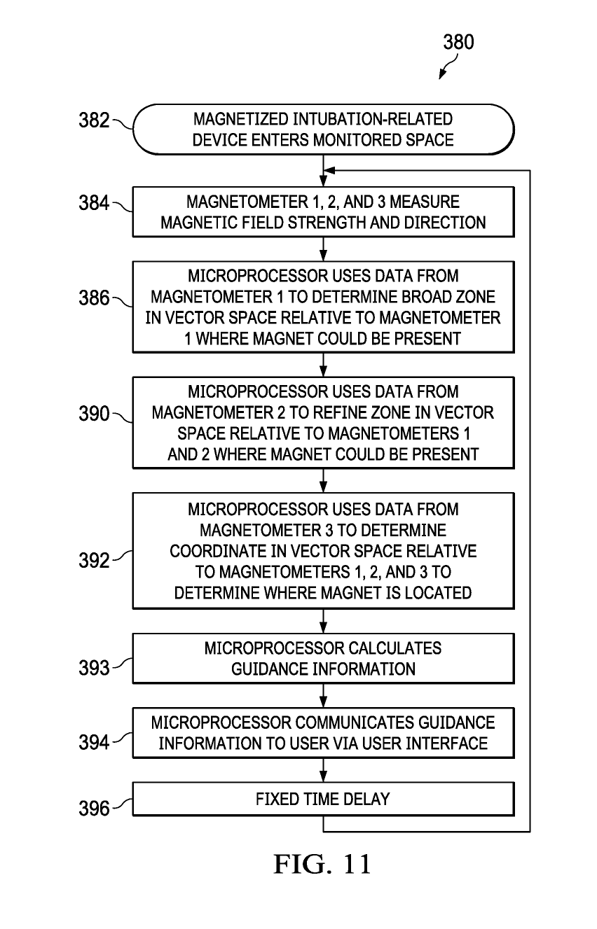

FIGS. 11-15 illustrate example algorithms, executable by data analysis system 28, for determining the three dimensional location of a stylet 44 relative to one or more Spatial Reference Elements (SREs) associated with a VL device/blade, from which data analysis system 28 may calculate “laterality,” “depth,” and “penetration” metrics and control LED array 30, according to example embodiments. The example algorithms shown in FIGS. 11-15 relate to an example magnet-based stylet guidance system 48 that includes (a) one or more magnetometers 24 associated with the VL device, for example, corresponding with one or more magnetometers 24A, 24B, 24C, . . . shown in any of the embodiments shown in any FIGS. 1-4, and (b) one or more stylet magnets 46 associated with stylet 44, e.g., at least one magnet region at or near the distal tip of stylet 44 or at least one magnet secured to stylet 44 at or near the distal tip of stylet 44.

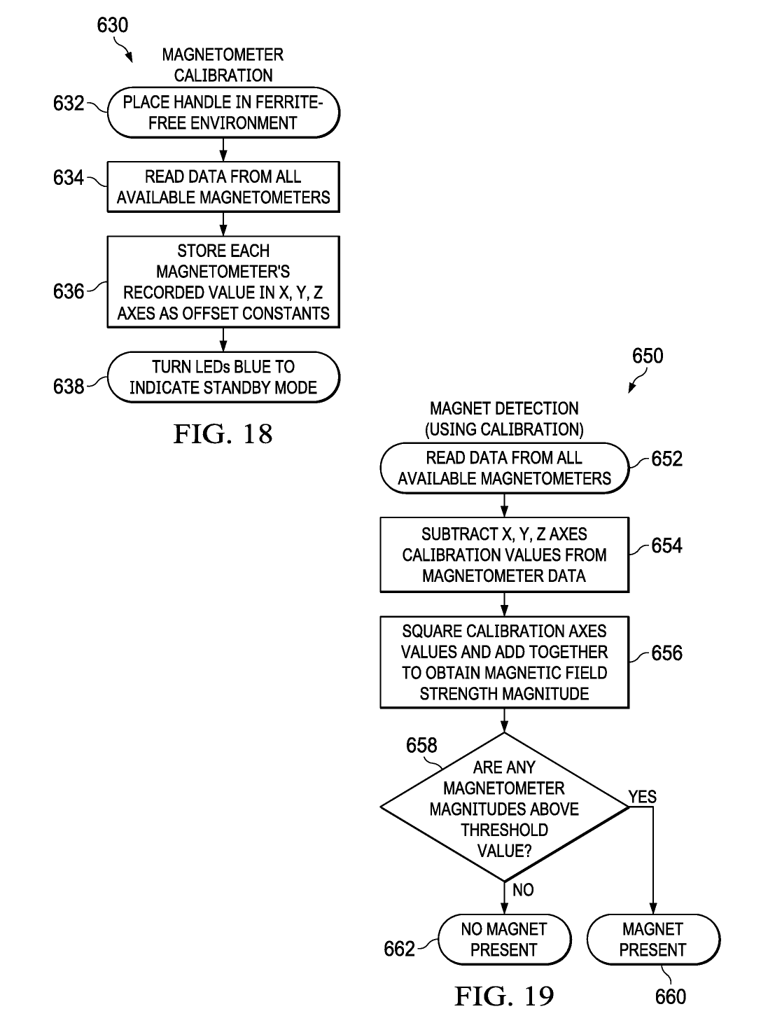

In some embodiments, each algorithm shown in FIGS. 11-15 may include or utilize the results of a magnetometer calibration process, e.g., using the calibration algorithm 630 shown in FIG. 18, discussed below.

FIG. 11 illustrates an example triangulation-based stylet location algorithm 380 executable by data analysis system 28, that utilizes triangulation techniques to determine the 3D location of stylet 44 (i.e., the 3D location of stylet magnet 46) using signals from at least three non-coplanar magnetometers 24 to detect a single stylet magnet 46, and provides guidance information to a user, e.g., via LED array 30, to facilitate an intubation procedure, according to an example embodiment. For example, with reference to example embodiments shown in FIGS. 2A and 4A, the triangulation algorithm 380 could use the laterally-facing magnetometer 24A, the laterally-facing magnetometer 24B, and the rear-facing magnetometer 24D, which are collectively non-coplanar with each other.

At 382, a styletted ETT 40 including stylet 44 with magnet 46 enters a space monitored by magnet-based stylet guidance system 48. At 384, each of magnetometers 1, 2, and 3 generates signals based on detected magnetic field strength and direction, and communicates such signals (data) to a processor of data analysis system 28.

At 386, the processor uses data from magnetometer 1 to determine a broad zone in vector space relative to magnetometer 1 where magnet 46 could be present. At 390, the processor uses data from magnetometer 2 to refine the zone in vector space relative to magnetometers 1 and 2 where magnet 46 could be present. At 392, the processor uses data from magnetometer 3 to determine coordinates in vector space relative to magnetometers 1, 2, and 3 where magnet 46 is located.

At 393, the processor may determine or calculate guidance information based on the magnet coordinates determined at 392. For example, the processor may calculate laterality, depth, and/or penetration values based on the determined magnet coordinates. At 394, the processor may communicate the guidance information to a user via a suitable guidance information output device. For example, the processor may control selected LEDs of the example LED array 30 shown in FIGS. 10A-10E to visually communicate the calculated laterality, depth, and/or penetration of stylet 44. As another example, the processor may display or otherwise output the calculated magnet coordinates in any suitable manner. The system may then implement a fixed time delay, e.g., in the range of 1-100 ms, and then repeat steps 384-394.

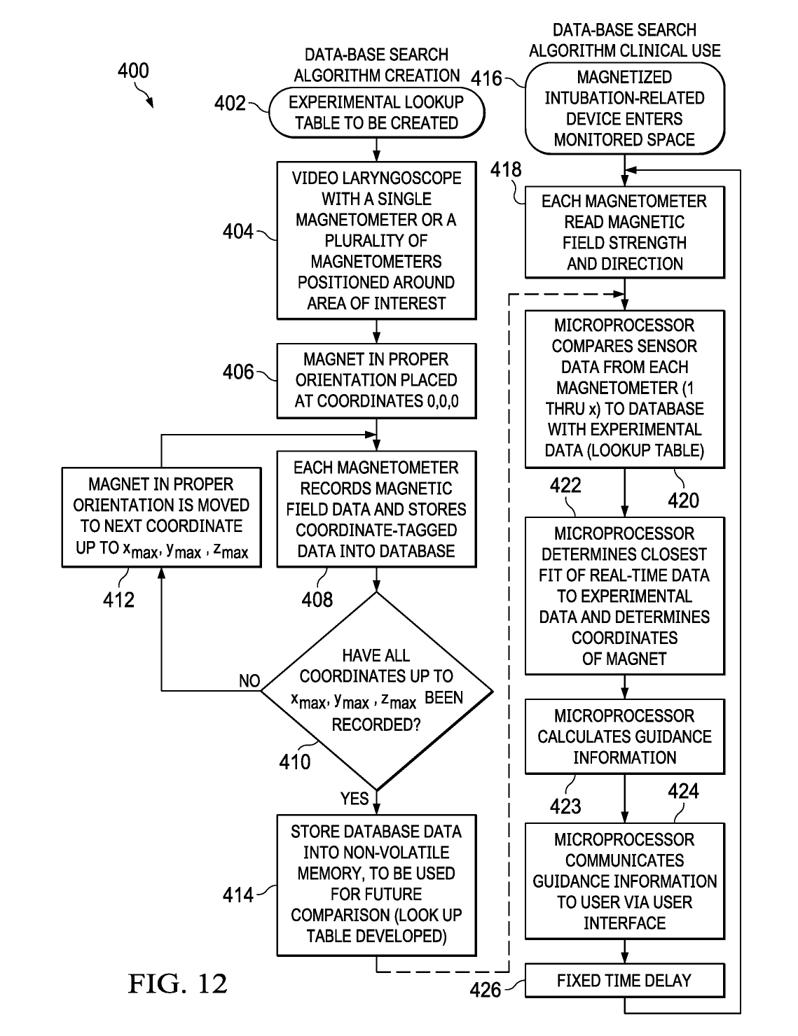

FIGS. 12-15 illustrate further algorithms for determining the location of a stylet magnet 46, without relying on the triangulation technique shown in FIG. 11. Thus, in some embodiments, effective execution of the algorithms shown in FIGS. 12-15 may not require at least three non-coplanar magnetometers 24. However, the quality of results of such algorithms may correspond with the number and arrangement of available magnetometers 24. For example, it may be advantageous to have an array of magnetometers 24 that spans the full range or area to be monitored, e.g., including a plurality of magnetometers 24 arranged along the full range of penetration to be monitored. Further, for monitoring a specific zone of stylet depth and/or laterality (e.g., the space to the left, right, or rear of the handle) it may be advantageous to arrange magnetometers 24 close to those zones so that signal interference from the VL handle itself may be reduced or minimized.

Further, some embodiments of guidance system 48 may include only one single magnetometer 24, and any suitable algorithms, e.g., algorithm 400 shown in FIG. 12 or any other algorithm disclosed herein—e.g., modified for use with a single magnetometer, where appropriate, using any suitable mathematical or data analysis techniques known by those skilled in the art—for determining stylet position information, e.g., a stylet location, orientation, movement velocity and/or direction, camera plane crossing status, etc.

FIG. 12 illustrate an example algorithm 400, executable by data analysis system 28, for generating a reference database of magnet location data and using the reference database for determining the current location of a stylet magnet 46, according to an example embodiment. Algorithm 400 may be executed for embodiments of stylet guidance system 48 including only a single magnetometer 24 or including multiple magnetometers 24. A database of reference magnet location data is generated at steps 402-414, and then utilized for determining the real-time location of a stylet 44 and providing guidance information to a user, e.g., via LED array 30, at steps 416-426, to facilitate an intubation procedure.

At 402, an experimental/reference database (e.g., lookup table) is created. A video laryngoscope including a single magnetometer 24 or a plurality of magnetometers 24 is provided at 404. At 406, a magnetized stylet 44 (e.g., stylet 44 including one or more magnets or magnetized regions 46) is placed in a proper orientation (e.g., corresponding to a typical intubation process) and at a location relative to the VL with defined x, y, z, coordinates of 0, 0, 0. At 408, each magnetometer 24 generates magnetic field data and the system stores coordinate-tagged data in the reference database created at 402. At 410, the system or user determines whether all 3D coordinates for the three-dimensional space to be mapped have been and evaluated and recorded (by arranging stylet 44 at the respective coordinates and recording magnetometer data in the reference database). If not, at 412 the stylet 44 is moved to the next coordinate set within the three-dimensional space to be mapped, and the magnetometer data for this coordinate set is recorded and stored as coordinate-tagged data in the reference database.

Once it is determined at 410 that all 3D coordinates in the three-dimensional space to be mapped have been evaluated and recorded, the reference database data may be stored in non-volatile memory of or accessible by a guided intubation system, e.g., in the form of a lookup table that associates magnetometer readings for the one or more magnetometers 24 with a corresponding three-dimensional coordinate (xᵢ, yᵢ, zᵢ).

The reference database (e.g., lookup table) may then be used by a magnet-based stylet guidance system 48 for determining the real-time location of a stylet 44 and providing guidance information to a user during an intubation procedure, at 416-426. At 416, a styletted ETT including stylet 44 with magnet(s) 46 enters a space monitored by magnet-based stylet guidance system 48. At 418, each magnetometer 24 of system 48 (e.g., a single magnetometer 24 or a plurality of magnetometers 24) reads magnetic field strength and direction data. At 420, a processor (e.g., provided by data analysis system 28 of guidance system 48) compares data from each magnetometer 24 (magnetometer 1 through magnetometer x) with reference data in the reference database (e.g., lookup table), and determines at 422 a best fit of the currently detected magnetometer data with the reference magnetometer data to identify a corresponding three-dimensional coordinate (xᵢ, yᵢ, zᵢ) of stylet 44.

At 423, the processor may determine or calculate guidance information based on the magnet coordinate determined at 422. For example, the processor may calculate laterality, depth, and/or penetration values based on the determined magnet coordinates. At 424, the processor may communicate the guidance information to a user via a suitable guidance information output device. For example, the processor may control selected LEDs of the example LED array 30 shown in FIGS. 10A-10E to visually communicate the calculated laterality, depth, and/or penetration of stylet 44. As another example, the processor may display or otherwise output the calculated magnet coordinates in any suitable manner. The system may then implement a fixed time delay at 426, e.g., in the range of 1-100 ms, and then repeat steps 418-426 as the stylet 44 is moved through 3D space during the intubation procedure.

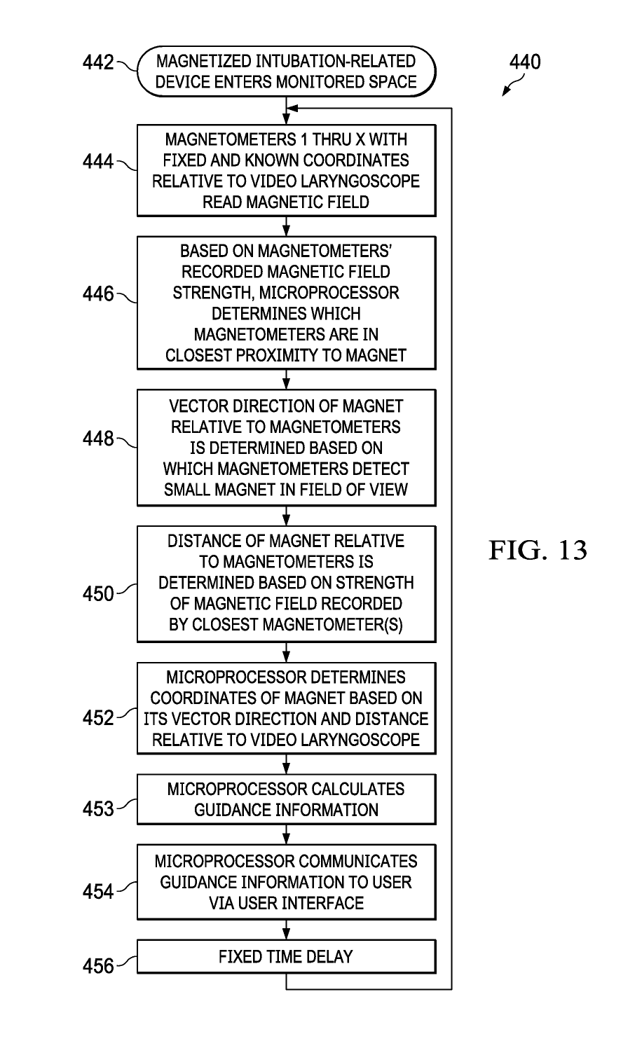

FIG. 13 illustrate an example stylet detection algorithm 440, executable by data analysis system 28, for determining a location of a magnetized stylet 44 and providing guidance information to a user, e.g., via LED array 30, to facilitate an intubation procedure, according to an example embodiment. In general algorithm 440 looks at the strongest magnetometer magnitude to determine a broad area in space for the stylet magnet 46, then looks at the next strongest magnetometer magnitude(s) to fine-tune the magnet location (i.e. if the next strongest magnetometer is at an adjacent penetration location, the system can determine that the magnet penetration is between two adjacent magnetometers or magnetometer “rings.” Further, if the next highest magnetometer magnitude is on the back face of the VL device, the system can determine a greater stylet depth. The system can continue the evaluation to provide an effective location determination/prediction.

At 442, a styletted ETT including stylet 44 with magnet(s) 46 enters a space monitored by magnet-based stylet guidance system 48. At 444, each available magnetometer 24 of system 48 (e.g., a single magnetometer 24 or a plurality of magnetometers 24) reads magnetic field strength and direction data. At 446, a processor (e.g., provided by data analysis system 28 of guidance system 48) analyzes the magnetometer data to determine which magnetometer(s) 24 are in a defined proximity to the magnet 46. At 448, the processor may determine a 2D or 3D vector direction of magnet 46 relative to one or more magnetometers 24 or other SRE associated with the VL device, based on the analyzed magnetometer data. At 450, the processor may determine a distance of magnet 46 relative to one or more magnetometers 24 or other SRE associated with the VL device, based on the magnitude of the closest proximity magnetometer(s) 24 identified at 446. At 452, the processor may determine 2D or 3D coordinates of magnet 46 based on the determined vector direction and distance between the magnet 46 magnetometer(s) 24 or other SRE associated with the VL device.

At 453, the processor may determine or calculate guidance information based on the magnet coordinates determined at 452. For example, the processor may calculate laterality, depth, and/or penetration values based on the determined magnet coordinates. At 454, the processor may communicate such guidance information to a user via a suitable guidance information output device, e.g., LED array 30. As another example, the processor may display or otherwise output the calculated magnet coordinates in any suitable manner. The system may then implement a fixed time delay at 456, e.g., in the range of 1-100 ms, and then repeat steps 444-454 as the stylet 44 is moved through 3D space during the intubation procedure.

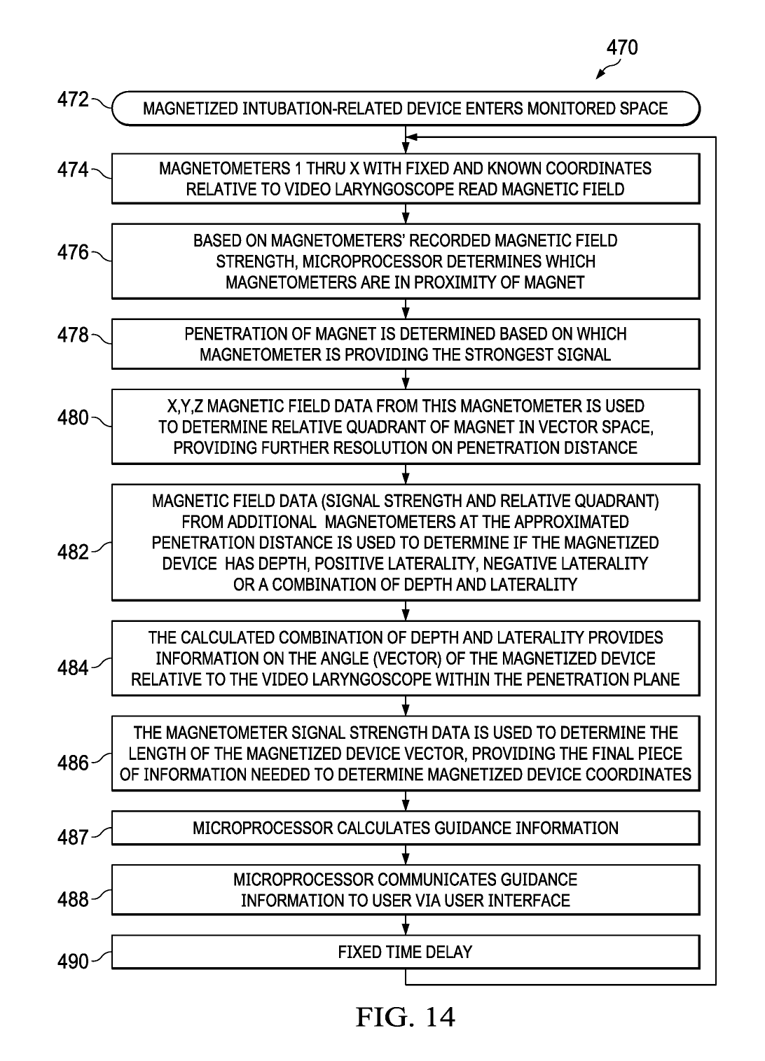

FIG. 14 illustrate another example stylet proximity detection algorithm 470, executable by data analysis system 28, for determining a location of a magnetized stylet 44 and providing guidance information to a user, e.g., via LED array 30, to facilitate an intubation procedure, according to an example embodiment. Data analysis system 28 may utilize algorithm 470 as an alternative to algorithm 440 shown in FIG. 13.

The orientation of magnet(s) 46 within a styletted ETT is generally restricted by physical constraints of the patient's trachea. Given this restricted orientation, it is known to one skilled in the art that a magnetometer's x, y, z magnetic field components will change sign as the magnet crosses a respective axis of the magnetometer. Data analysis system 28 may use this information to determine a relative quadrant (e.g. in the x-y plane) of a detected magnet 46.

Algorithm 470 may utilize such knowledge and use the magnetometer magnetic field data in each of the three axes (each axis has positive and negative value). Knowing the properties of a magnetic field, the system can use this data to find the location of a magnet 46 relative to a magnetometer 24. With one strong magnet 46, a single magnetometer 24 could be used to detect the magnet location. However, in may be more effective to use a smaller/weaker magnet 46 and multiple magnetometers 24, especially because data from multiple magnetometers 24 can be combined to increase the confidence level of a location determination/prediction.

At 472, a styletted ETT including stylet 44 with magnet(s) 46 enters a space monitored by magnet-based stylet guidance system 48. At 474, each available magnetometer 24 of system 48 (e.g., a single magnetometer 24 or a plurality of magnetometers 24) reads magnetic field strength and direction data. At 476, a processor (e.g., provided by data analysis system 28 of guidance system 48) analyzes the magnetometer data to determine which magnetometer(s) 24 are in a defined proximity to the magnet 46. At 478, the processor may determine a penetration of magnet 46 based on the highest magnitude magnetometer 24.

At 480, the processor may analyze the x, y, z magnetic field data from the highest magnitude magnetometer 24 to determine a relative quadrant of magnet 46 in vector space, thus providing further resolution of the penetration distance/extent. At 482, the processor may analyze magnetic field data (e.g., signal strength and relative quadrant data) from additional magnetometer(s) 24 to the approximated penetration distance/extent to determine the combination of stylet depth and laterality at that penetration distance/extent. As indicated at 484, the calculated combination of depth and laterality provides information regarding the angle (vector) of the magnet 46 relative to the VL device or relevant SRE within the penetration plane. At 486, the processor uses the magnetometer signal strength data to determine the length of the magnet 46 vector, thus providing the final piece of information needed to determine the 3D magnet coordinates.

At 487, the processor may determine or calculate guidance information based on the magnet coordinates determined at 486. For example, the processor may calculate laterality, depth, and/or penetration values based on the determined magnet coordinates. At 488, the processor may communicate such guidance information to a user via a suitable guidance information output device, e.g., LED array 30. As another example, the processor may display or otherwise output the calculated magnet coordinates in any suitable manner. The system may then implement a fixed time delay at 490, e.g., in the range of 1-100 ms, and then repeat steps 474-488 as the stylet 44 is moved through 3D space during the intubation procedure.

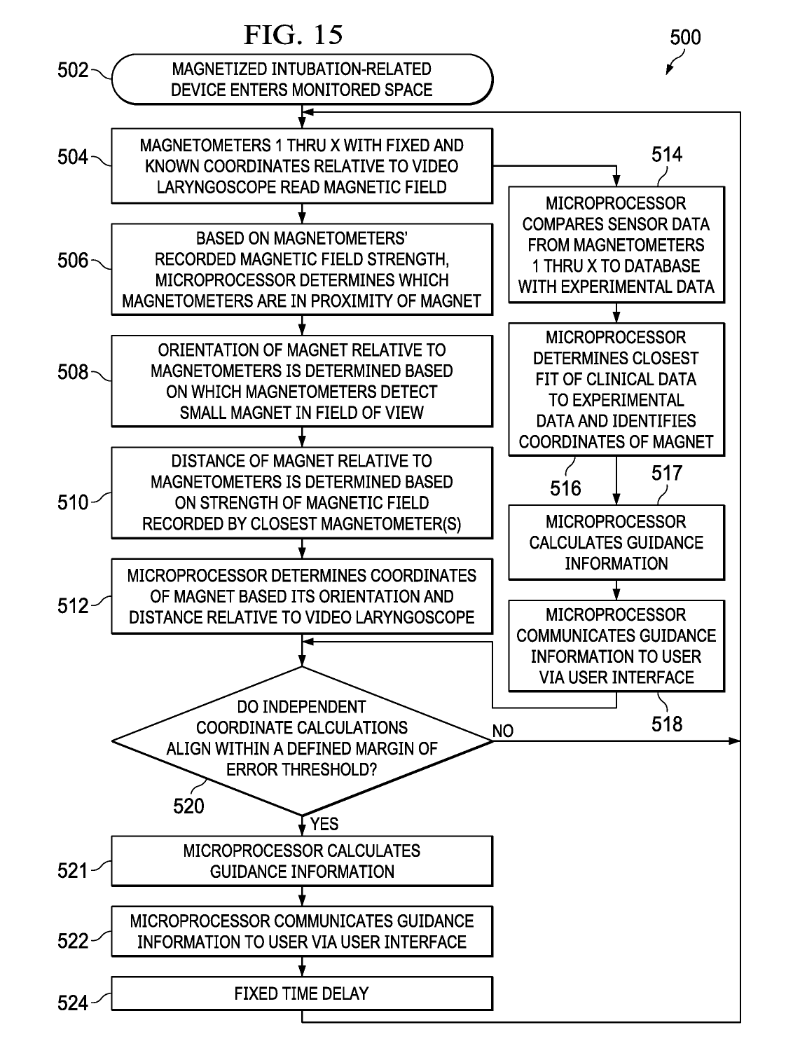

FIG. 15 illustrate another example stylet proximity detection algorithm 500, executable by data analysis system 28, for determining a location of a magnetized stylet 44 and providing guidance information to a user, e.g., via LED array 30, to facilitate an intubation procedure, according to an example embodiment. Algorithm 500 is essentially a hybrid of algorithm 400 shown in FIG. 12 and algorithm 440 shown in FIG. 13. Data analysis system 28 may utilize algorithm 500 as an alternative to algorithm 440 shown in FIG. 13 or algorithm 470 shown in FIG. 14.

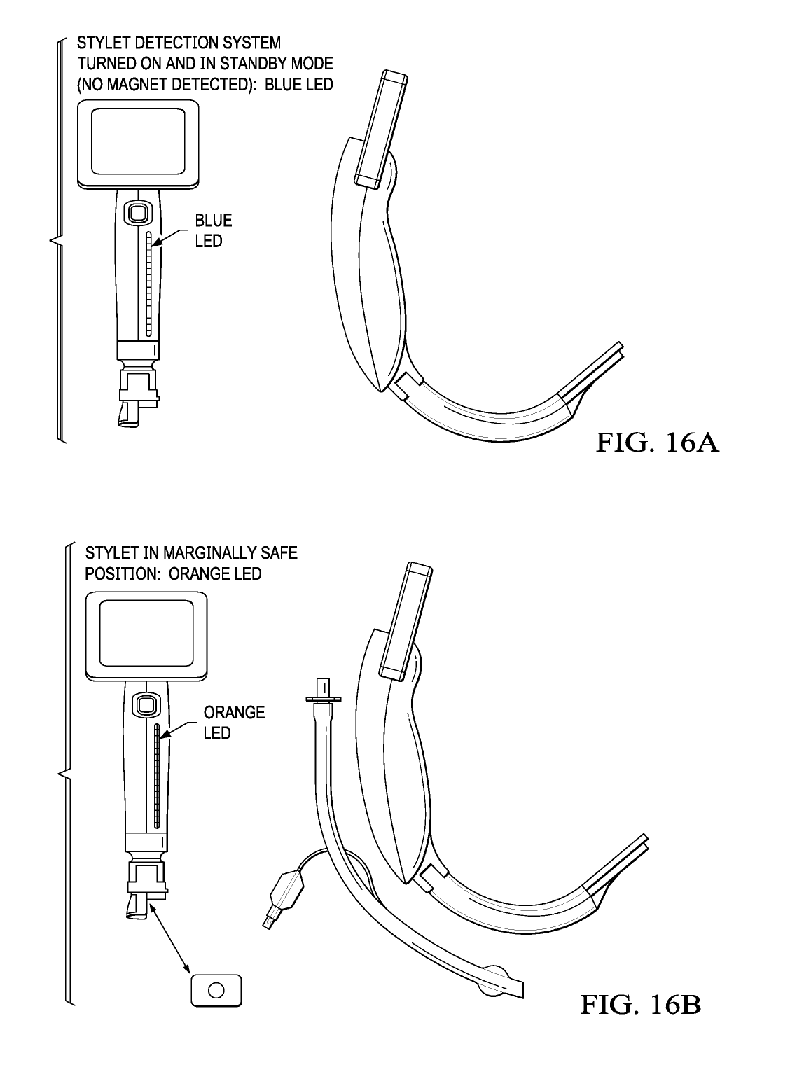

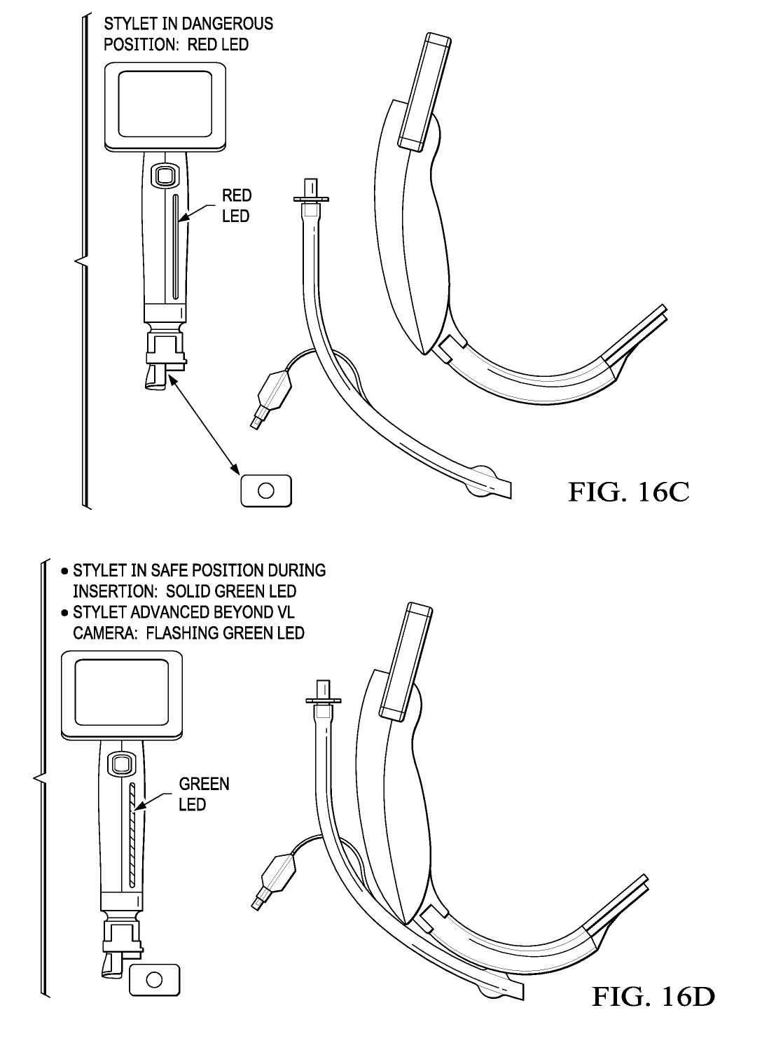

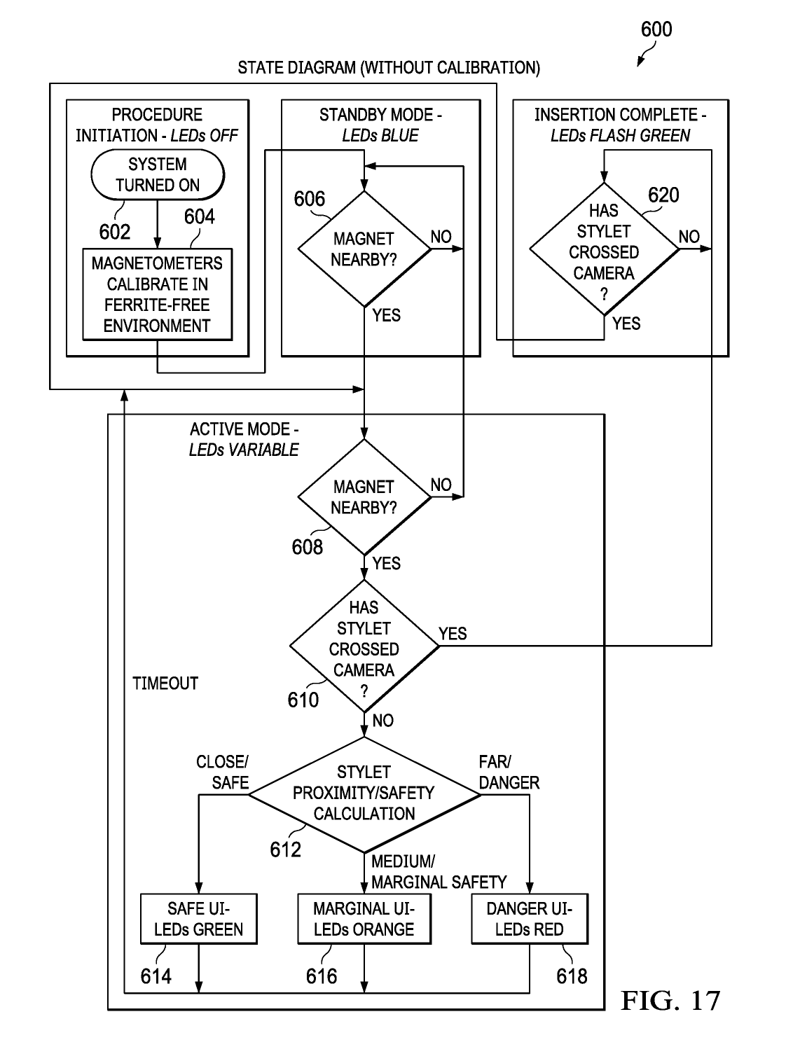

FIGS. 16A-16D illustrate another example guidance information display 30 for providing guidance information to a proceduralist, by indicating a current state of the guidance system, according to example embodiments. This example guidance information display 30 includes a series of colored LEDs 30 (or a single or multiple multi-colored LEDs) integrated in, attached to, or otherwise located on or at the handheld VL device, e.g., on the VL handle. The proceduralist may be instructed to use the video laryngoscope as they normally would and keep their attention focused “in the mouth.” As the user inserts a styletted endotracheal tube 40, data analysis system 28 may determine and monitor position information regarding the stylet 44 relative to one or more Stylet Reference Elements (SREs) associated with the VL device, e.g., using any techniques or algorithms disclosed herein (e.g., any of the algorithms shown in FIGS. 17-29 and discussed below, and/or any of the algorithms shown in FIGS. 11-15 discussed above). Data analysis system 28 then control the guidance information display 30 shown in FIGS. 16A-16D based on the determined stylet position information, e.g., in the following manner:

1. When the stylet guidance system 48 is turned on and calibrated and enters the standby mode, i.e., stylet 44 is not yet detected, the LED(s) 30 are illuminated blue, as shown in FIG. 16A.

2. When system 48 determines that stylet 44 is in a marginally safe position, the LED(s) 30 are illuminated yellow, as shown in FIG. 16B.

3. When system 48 determines that stylet 44 is in an unsafe position, the LED(s) 30 are illuminated red, as shown in FIG. 16C.

4. When system 48 determines that stylet 44 is in a safe position (e.g., close to the VL blade, and thus on a proper course for intubation), the LED(s) 30 are illuminated green, as shown in FIG. 16D.

5. Once stylet 44 has been inserted beyond the VL camera 20, the LED(s) 30 blink/flash green, which indicates to the proceduralist that they may look at VL video screen 23 for continued assistance with the intubation procedure.

In an alternative embodiment, LED(s) 30 are illuminated blue during the standby mode and also when stylet 44 is detected in an unsafe or marginal state, illuminated solid green when stylet 44 is detected in safe state, and flashing green when stylet 44 is inserted beyond the VL camera plane. In this embodiment, the system provides only positive feedback (via green illumination) and not negative feedback.

FIGS. 17-29 illustrate various algorithms that may be executed by an intubation guidance system for calibrating the system, detecting and monitoring the location and/or orientation of an endotracheal stylet 44, and displaying information defining a current state of the guidance system, e.g., via the guidance information display 30 shown in FIGS. 16A-16D, based at least on the determined stylet location and/or orientation.

FIG. 17 illustrates an example algorithm 600 for providing guidance-based facilitation of an intubation procedure using a magnet-based stylet guidance system 48, according to an example embodiment. Algorithm 600 is a state-based algorithm for monitoring the current state of magnet-based stylet guidance system 48 and providing guidance output via the example state-based guidance information display 30 shown in FIGS. 16A-16D, according to example embodiments. In general, data analysis system 28 of stylet guidance system 48 may analyze magnetometer data to determine position information regarding a magnetized stylet 44, determine a current state of stylet guidance system 48 based at least on the determined stylet position information, and output such state information via guidance information display 30 shown in FIGS. 16A-16D. Each decision step in algorithm 600 may be performed or facilitated by one or more stylet-position-based algorithms shown in FIGS. 18-29.

Referring to algorithm 600, in a “procedure initiation mode,” at step 602 a VL system 12 and magnet-based stylet guidance system 48 (integrated with or separate from VL system 12) are turned on. At 604, a magnetometer calibration is performed, e.g., in a ferrite-free environment. In one embodiments, stylet guidance system 48 may execute magnetometer calibration algorithm 630 shown in FIG. 18, which is discussed below. While system 48 is in the procedure initiation mode, the guidance LED display 30 shown in FIGS. 16A-16D remain off (non-illuminated).

After the magnetometer calibration, system 48 may enter a “standby mode,” wherein the guidance LED display 30 is illuminated blue. In the standby mode, stylet guidance system 48 determines whether stylet magnet 46 is nearby at 602. In one embodiments, stylet guidance system 48 may execute magnet detection algorithm 650 shown in FIG. 19, which is discussed below. Stylet guidance system 48 may continue to detect stylet magnet 46 nearby, e.g., by repeated execution of algorithm 650. Once stylet guidance system 48 detects a nearby magnet 46, the system may enter an “active mode,” in which system 48 evaluates the current position/camera plane crossing status of stylet 44 (via magnet 46), and controls the color of LED display 30 based on such determinations.

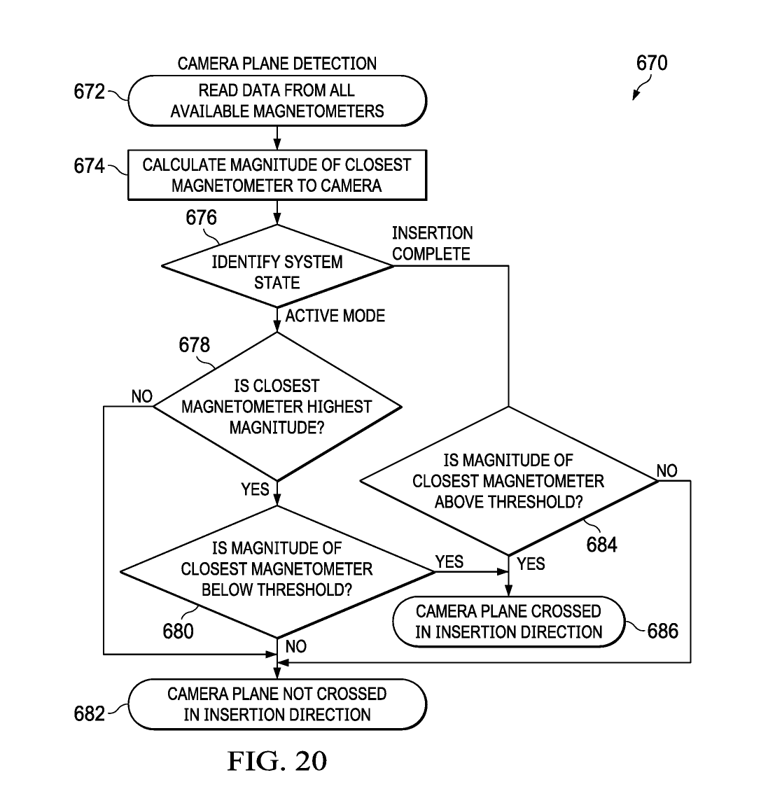

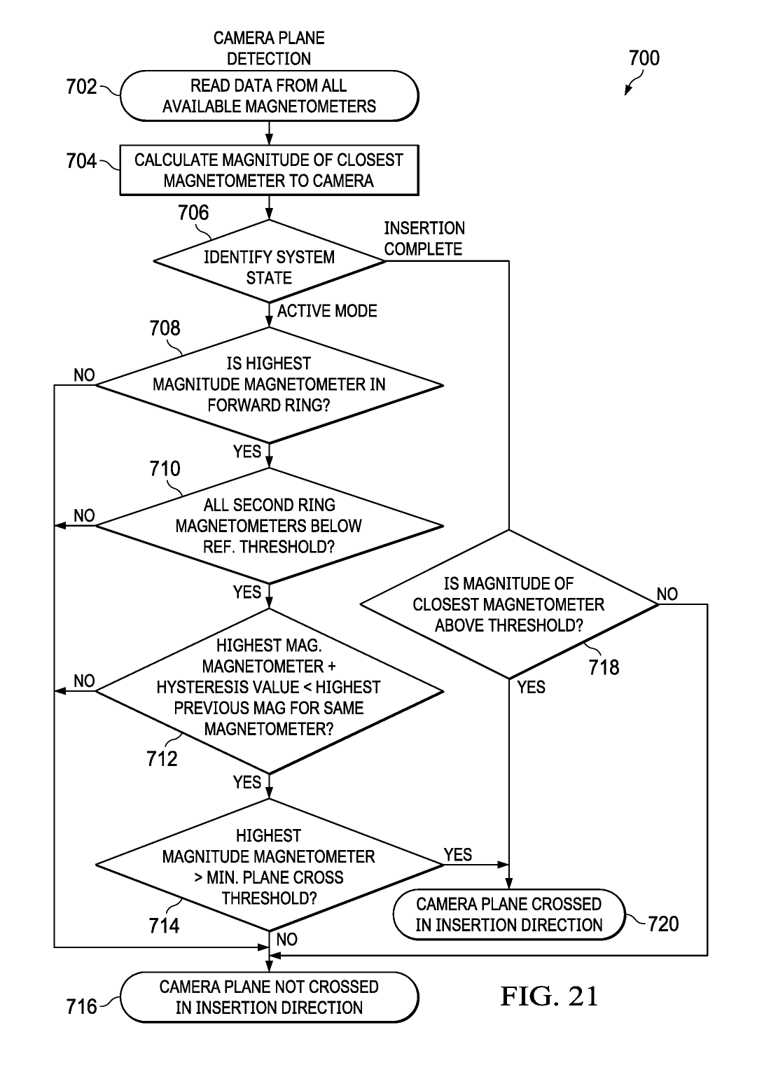

At 608, system 48 confirms that magnet 46 is nearby, e.g., using algorithm magnet detection algorithm 650 shown in FIG. 19. If not, the system 48 switches back to the standby mode, and LED display 30 is illuminated blue, during continued attempts to detect a nearby magnet 46. If system 48 confirms that magnet 46 is nearby, system 48 may determine whether stylet 44 has crossed the VL camera plane in an insertion direction at 610, e.g., by executing algorithm 670 shown in FIG. 20 or algorithm 700 shown in FIG. 21. If system 48 determines that stylet 44 has crossed the VL camera plane at 610, system 48 may enter an “insertion complete mode” in which LED display 30 is illuminated green. In the insertion complete mode, system 48 may (repeatedly) detect whether stylet 44 has crossed the VL camera plane in a withdrawal (upstream) direction at 620, e.g., by executing step 684 of algorithm 670 (FIG. 20) or step 718 of algorithm 700 (FIG. 21). If system 48 detects a withdrawal-direction camera plane crossing, system 48 may return to the “active mode” as shown in FIG. 17. Otherwise, system 48 may remain in the insertion complete mode.

Returning to step 610, system 48 determines that stylet 44 has not crossed the VL camera plane, system 48 may perform a stylet proximity or safety determination at 612 to determine one of a “safe” state, “marginal” state, or “danger” state, and control LED display 30 based on the results. In this example, system 48 illuminates LED display 30 green for a determined “safe” state at 614, orange for a determined “marginal” state at 616, or red for a determined “danger” state at 618. After performing the stylet proximity or safety determination at 612, the system may repeat the active mode steps 608, 610, and 612, to continuously monitor the state of stylet 44 and provide relevant feedback via LED display 30.

FIG. 18 illustrates an example magnetometer calibration algorithm 630 to calibrate magnetometers 24 located in/on the handheld VL device, which may be executed by stylet guidance system 48 (in particular, by data analysis system 28) at step 604 of state-based algorithm 600 shown in FIG. 17, according to one embodiment. At 632, a user may place the handheld VL device including magnetometers 24 in a ferrite-free environment, e.g., away from magnetized stylet 44 and other magnetized or ferrite-containing structures. At 634, system 48 may read data from all available magnetometers 24. At 636, system 48 may store the recorded data from each magnetometer 24 in the x, y, and z axes as offset constants. At 636, upon completion of the calibration, system 48 may enter the standby mode (see FIG. 17) and accordingly illuminate LED display 30 blue.

FIG. 19 illustrates an example magnet detection algorithm 650 for detecting whether a magnet 46 is nearby, which may be executed by stylet guidance system 48 (in particular, by data analysis system 28) at step 606 and 608 of state-based algorithm 600 shown in FIG. 17, according to one embodiment. At 652, system 48 may read data from all available magnetometers 24. At 654, system 48 may subtract x, y, and z axes calibration values (determined via algorithm 630) from the magnetometer data. At 656, system 48 may square and sum the calibration axes values for each magnetometer to calculate a magnetic strength magnitude for each magnetometer. At 658, system 48 may compare the magnetic strength magnitude for each magnetometer to a threshold value. If at least one of the magnetometer magnitudes exceeds the threshold value, the system 48 determines that a magnet is nearby at 660. Otherwise, if none of the magnetometer magnitudes exceed the threshold value, the system 48 determines that no magnet is nearby at 662.

FIGS. 20 and 21 illustrate two example camera plane detection algorithms 670 and 700 for determining whether stylet 44 (via magnet 46) has crossed a VL camera plane (e.g., camera plane CP shown in FIG. 1) in an insertion (downstream) and/or withdrawal (upstream) direction, according to example embodiments. For example, algorithm 670 or 700 may be executed by stylet guidance system 48 (in particular, by data analysis system 28) at step 610 (insertion-direction camera plane crossing) and step 620 (withdrawal-direction camera plane crossing) of state-based algorithm 600 shown in FIG. 17.

FIG. 20 illustrates a first example camera plane detection algorithm 670, according to one embodiment. At 672, system 48 may read data from all available magnetometers 24. At 654, system 48 may determine the magnitude of a selected “forward magnetometer 24,” which may be the magnetometer or magnetometer ring closest to camera 20 or closest to the distal tip of the VL blade, e.g., magnetometer or magnetometer ring 24A shown in any of FIGS. 1-4. At 674, system 48 may identify the current system state, e.g., with reference to state-based algorithm 600 shown in FIG. 17.

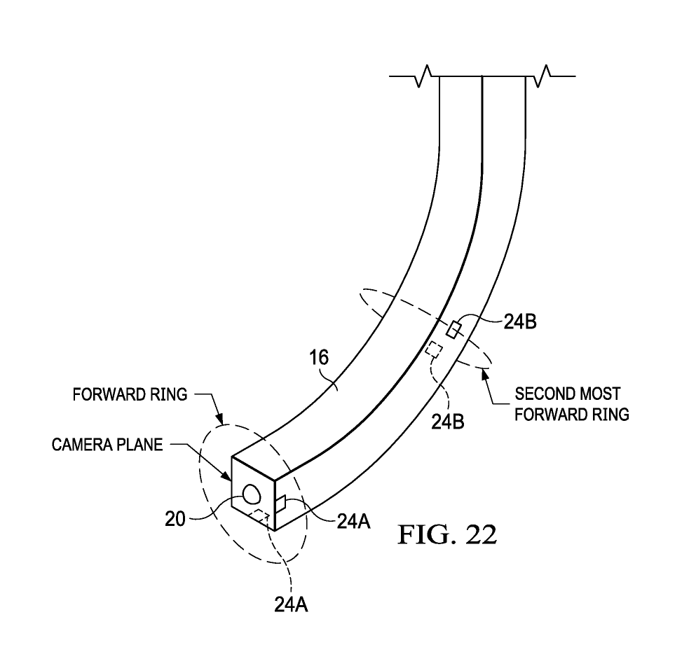

If the system is currently in the “active mode” (see FIG. 17, step 610), system 48 may execute steps 678 and 680 to detect an insertion-direction camera plane crossing event. At 678, system 48 determines whether a forward magnetometer 24 (e.g., either magnetometer in a forward magnetometer ring including two magnetometers 24, such as shown in FIG. 22, for example) has the highest magnitude of all available magnetometers 24. If not, this indicates that the magnet 46 is closer to an upstream magnetometer 24, and thus system 48 determines that stylet 44 has not crossed the camera plane in an insertion direction, at 682. Alternatively, if the forward magnetometer 24 does have the highest magnitude of all available magnetometers 24, the method proceeds to 680, where system 48 determines whether the magnitude of the forward magnetometer 24 is below a defined threshold value. If so, system 48 identifies that stylet 44 has crossed the camera plane in an insertion (downstream) direction, at 686. If not, system 48 determines that stylet 44 has not crossed the camera plane the an insertion direction, at 682.

Returning to step 674, if the system is currently in the “intubation complete mode” (see FIG. 17, step 620), system 48 may proceed to step 684 to detect a withdrawal-direction camera plane crossing. For example, at 684, system 48 may determine whether the magnitude of the forward magnetometer 24 exceeds a defined threshold value, which may be the same or different threshold value as used in step 680. If the forward magnetometer 24 magnitude exceeds a defined threshold value, system 48 identifies that stylet 44 has crossed the camera plane in a withdrawal direction, indicated in this instance at 682, and system 48 may return to the “active mode” as indicated in FIG. 17 by the “yes” decision at step 620, and may change the LED display color accordingly. If the forward magnetometer 24 magnitude does not exceed the defined threshold value, system 48 identifies that stylet 44 has not crossed the camera plane in the withdrawal direction, i.e., stylet 44 remains forward of the camera plane.

FIG. 21 illustrates a second example camera plane detection algorithm 700, according to one embodiment, and relates to the example magnetometer arrangement shown in FIG. 22, or other similar magnetometer arrangement. As shown in FIG. 22, a VL blade 16 may include (at least) two magnetometer rings arranged along the length of the VL blade 16. Each magnetometer ring may include one, two, or more magnetometers arranged in the same transverse plane, and may define a magnetic field “ring.” In this embodiment, two magnetometer rings are shown, (1) a forward ring including a forward pair of magnetometers 24A, e.g., at or near the camera plane defined by VL camera lens 20 and (2) and a second-most-forward ring including a pair of magnetometers 24B arranged along the VL blade 16 at a location set back from the forward ring. The forward ring may be co-planar or parallel with the camera plane to be detected for stylet crossings.

Algorithm 700 may be essentially similar to algorithm 670 shown in FIG. 20, except the “active mode” analysis steps 676-678 of algorithm 670 are replaced by “active mode” analysis steps 708-714 in algorithm 700. At 706, system 48 may determine whether one of magnetometers 24A in the forward ring has the highest magnetic magnitude of all magnetometers 24A and 24B (e.g., wherein the magnetic magnitude of each magnetometer 24A, 24B is determined based on a root square of the detected x, y, and z magnetic fields).

If yes, the method proceeds to 708, where system 48 may determine whether all magnetometers 24B in the second-most-forward ring are all below a reference threshold, thus indicating that stylet 44 is between the camera plane and the forward ring, and not between the forward ring and the second-most-forward rings.

If yes, the method proceeds to 708, where system 48 may determine whether the magnitude of the highest magnitude magnetometer 24A plus a defined hysteresis value is lower than the highest value previously calculated for that magnetometer, thus indicating that the magnet 44 has already passed the plane of the forward ring and is traveling past the camera plane in the insertion direction. In one embodiment, the hysteresis value is adjusted as a function of the distance between the forward ring plane and the camera plane.

If yes, the method proceeds to 708, where system 48 may determine whether the magnitude of the highest magnitude magnetometer 24A is greater than a defined minimum plane cross threshold. This may ensure that a plane cross event is not triggered when the stylet 44 is too far from the VL blade 16, as the focus should remain on bringing the stylet 44 closer to the VL blade 16 before shifting attention to VL video screen 23.

If yes (i.e., if all determinations at steps 708-714 provide a yes response), system 48 identifies that stylet 44 has crossed the camera plane in the insertion (downstream) direction, at 720. If any of the determinations at step 708-714 provide a no response, system 48 determines that stylet 44 has not crossed the camera plane the insertion direction, at 716.

In addition, in one embodiment, step 718 for identifying a withdrawal-direction camera plane crossing (while the system is in the “insertion complete mode”) may be replaced by an evaluation of steps 708 and 710. If both determinations at 708 and 710 provide a “no” result, system 48 determines that stylet 44 has crossed the camera plane in the withdrawal direction, and the system may return to the “active mode” as indicated in FIG. 17 by the “yes” decision at step 620, and may change the LED display color accordingly.

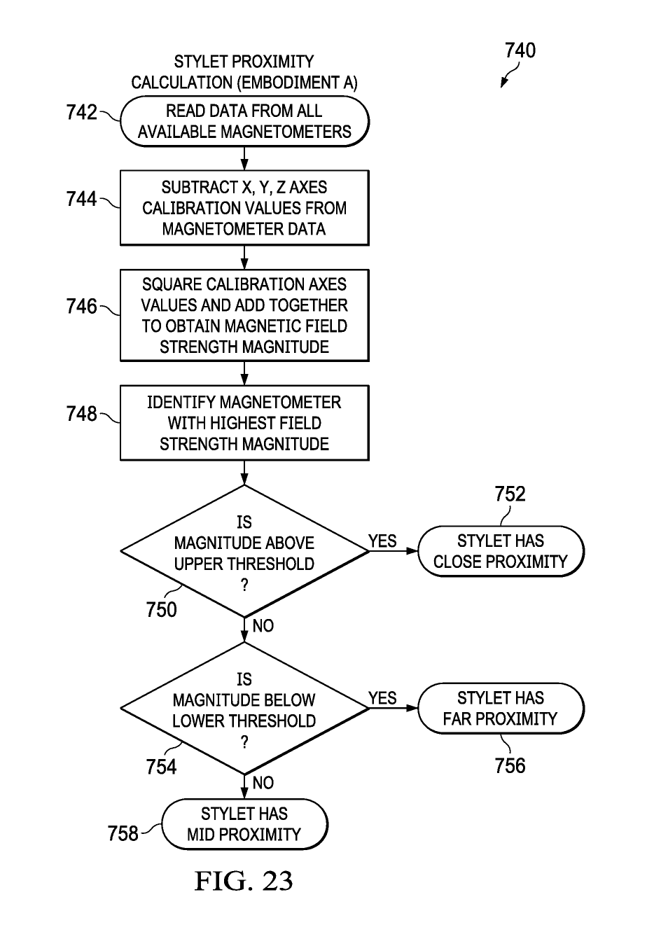

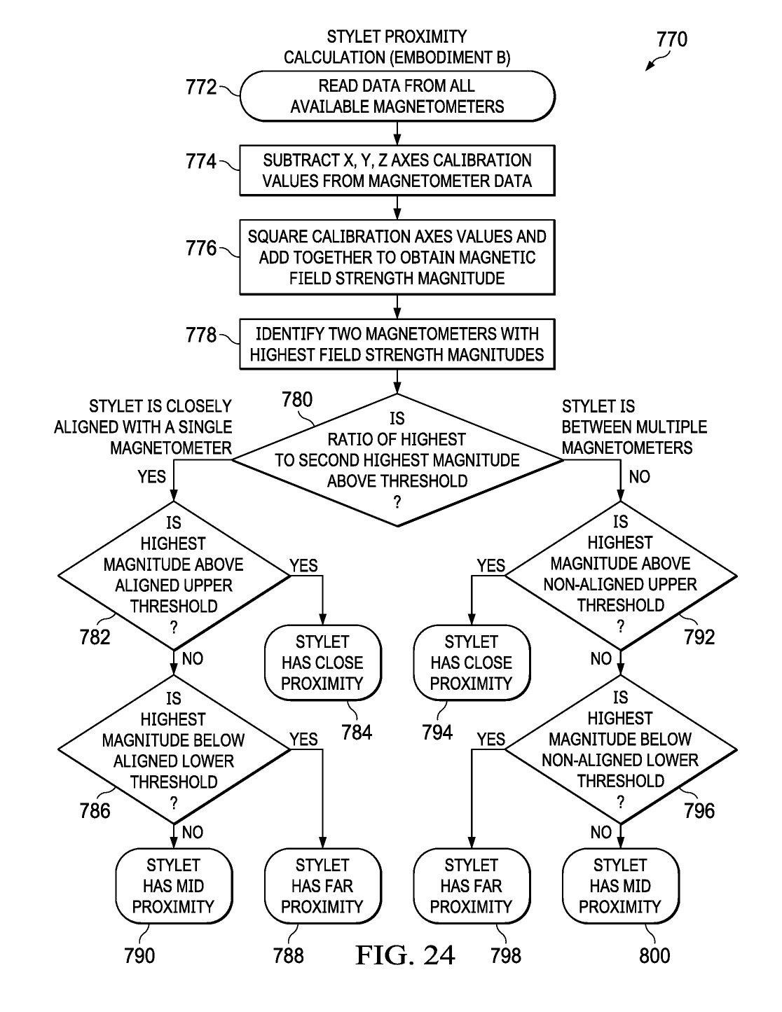

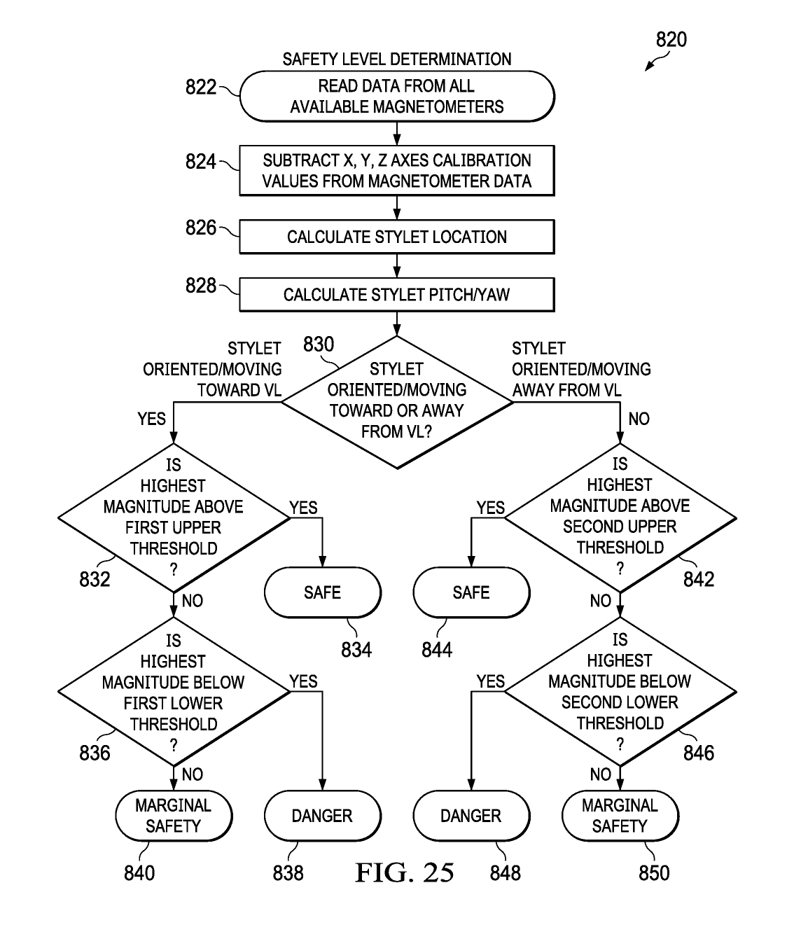

FIGS. 23 and 24 illustrate two example algorithms 740 and 770 for calculating a stylet proximity metric (e.g., with respect to one or more magnetometers 24 or other SREs associated with the VL device), according to example embodiments. FIG. 25 illustrates an example algorithm 820 for calculating a safety level, based on detected stylet location and orientation (e.g., pitch/yaw), according to an example embodiment. Algorithm 740, 770, or 820 may be executed by stylet guidance system 48 (in particular, by data analysis system 28) at step 612 of state-based algorithm 600 shown in FIG. 17, to determine stylet proximity or safety level data used for controlling LED display 30 (at step 614, 616, or 618).

Turning first to FIG. 23, stylet proximity algorithm 740 is executable to calculate a stylet proximity metric, e.g., with respect to one or more magnetometers 24 or other SREs associated with the VL device, according to an example embodiment. At 742, system 48 may read data from all available magnetometers 24. At 744, system 48 may subtract x, y, and z axes calibration values (e.g., determined via algorithm 630) from the magnetometer data. At 746, system 48 may square and sum the calibration axes values for each magnetometer to calculate a magnetic strength magnitude for each magnetometer.

At 748, system 48 may identify the magnetometer 24 having the highest field strength magnitude. At 750, system 48 may determine whether the highest field strength magnitude exceeds a defined upper threshold value. If so, system 48 determines that the stylet 44 has a close proximity, at 752. If not, the method may proceed to 754, where system 48 may determine whether the highest field strength magnitude is below a defined lower threshold value (which is lower than the defined upper threshold value). If so, system 48 determines that the stylet 44 has a far proximity, at 756. If not, system 48 determines that the stylet 44 has a medium proximity, at 758.

FIG. 24 illustrates another stylet proximity algorithm 770 executable to calculate a stylet proximity metric, e.g., with respect to one or more magnetometers 24 or other SREs associated with the VL device, according to an example embodiment. Stylet proximity algorithms 740 and 770 may represent alternatives to each other. At 772, system 48 may read data from all available magnetometers 24. At 774, system 48 may subtract x, y, and z axes calibration values (e.g., determined via algorithm 630) from the magnetometer data. At 776, system 48 may square and sum the calibration axes values for each magnetometer to calculate a magnetic strength magnitude for each magnetometer.

At 778, system 48 may identify the two magnetometers 24 having the highest field strength magnitudes of all available magnetometers 24. At 780, system 48 may determine whether the ratio of the highest magnetometer magnitude to the second highest magnetometer magnitude exceeds a defined threshold ratio. If the calculated ratio exceeds the threshold, system 48 determines that the stylet 44 is closely aligned with a single magnetometer 24, and proceeds to step 782. If the calculated ratio does not exceed the threshold, system 48 determines that the stylet 44 is between multiple magnetometers 24, and proceeds to step 792.

For a “closely aligned” determination at 780, the method proceeds to 782. At 782, system 48 determines whether the highest magnetometer magnitude exceeds an aligned-stylet upper threshold value. If so, system 48 determines that the stylet 44 has a close proximity, at 784. If not, the method proceeds to 786, where system 48 determines whether the highest field strength magnitude is below an aligned-stylet lower threshold value (which is lower than the aligned-stylet upper threshold value). If so, system 48 determines that the stylet 44 has a far proximity, at 788. If not, system 48 determines that the stylet 44 has a medium proximity, at 790.

For a “between two magnetometers” (i.e., not closely aligned) determination at 780, the method proceeds to 792. At 792, system 48 determines whether the highest magnetometer magnitude exceeds a non-aligned-stylet upper threshold value. If so, system 48 determines that the stylet 44 has a close proximity, at 794. If not, the method proceeds to 796, where system 48 determines whether the highest field strength magnitude is below a non-aligned-stylet lower threshold value (which is lower than the non-aligned-stylet upper threshold value). If so, system 48 determines that the stylet 44 has a far proximity, at 798. If not, system 48 determines that the stylet 44 has a medium proximity, at 800.

In one embodiment, the aligned-stylet upper threshold value and aligned-stylet lower threshold value are each higher than the respective non-aligned-stylet upper threshold value and non-aligned-stylet lower threshold value, as higher magnetometer readings are expected in an aligned position of the stylet.

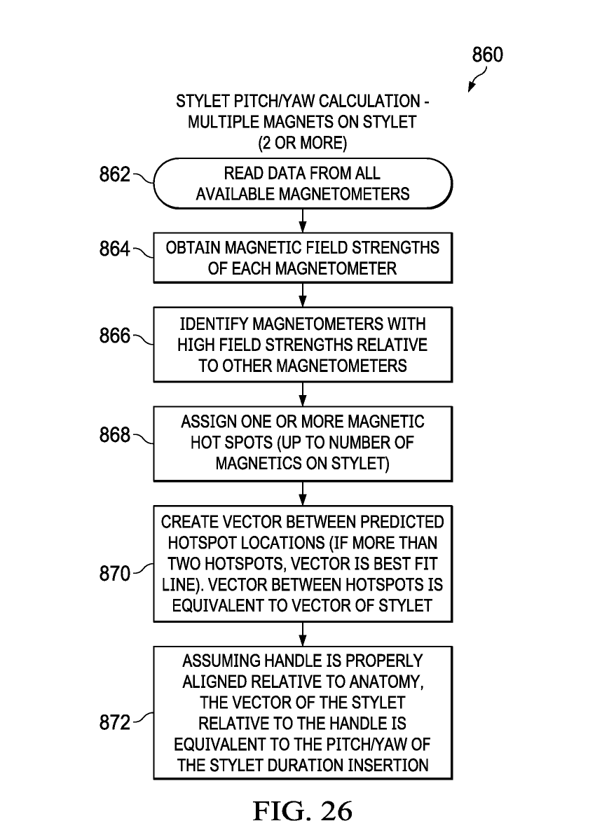

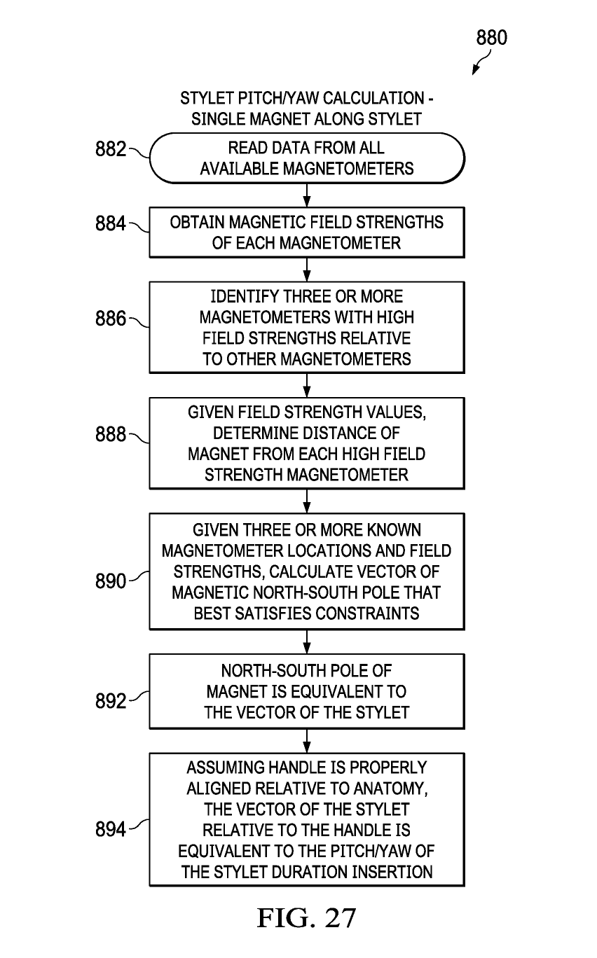

FIG. 25 illustrates an example algorithm 820 for calculating a safety level, based on detected stylet location and orientation (e.g., pitch/yaw), according to an example embodiment. As discussed above, system 48 may execute algorithm 820 at step 612 of algorithm 600 shown in FIG. 17. As discussed below, step 828 of algorithm 820 involves calculating angular orientation data regarding stylet 44, e.g., pitch and/or yaw data calculated using algorithm 860 shown in FIG. 26 or algorithm 880 shown in FIG. 27. As algorithm 860 relies on data from multiple stylet magnets 46, embodiments of stylet guidance system 48 that utilize algorithm 860 as input for algorithm 820, which in turn may be used as input for algorithm 600 shown in FIG. 17 (step 612) may include multiple magnets or magnetized regions 46 arranged along the length of stylet 44. Embodiments that utilize algorithm 880 for calculating pitch/yaw data may include a single stylet magnet 46.

At 822, system 48 may read data from all available magnetometers 24. At 824, system 48 may subtract x, y, and z axes calibration values (e.g., determined via algorithm 630) from the magnetometer data.

At 826, system 48 may calculate or determine a three-dimensional location of stylet 44, e.g., the location of the stylet magnet 46 (in embodiments that include a single magnet 46) or the location of the leading stylet magnet 46 (in embodiments that include multiple stylet magnets 46). System 48 may calculate or determine the three-dimensional location of stylet 44 using any of the algorithms or techniques disclosed herein, e.g., using any of the algorithms shown in FIGS. 11-15.

At 828, system 48 may calculate angular orientation data regarding stylet 44, e.g., the pitch and/or yaw of stylet 44, using algorithm 860 shown in FIG. 26 or algorithm 880 shown in FIG. 27, as discussed above. At 830, system 48 may determine whether stylet 44 is oriented or moving toward or away from the VL blade or relevant SRE, based on the stylet location calculated at 826 and the angular orientation (pitch/yaw) data calculated at 828.

If stylet 44 is oriented or moving toward the VL blade or relevant SRE, the method proceeds to step 832. Alternatively, if stylet 44 is oriented or moving away from the VL blade or relevant SRE, the method proceeds to step 842.

The situation in which stylet 44 is oriented or moving toward the VL blade or relevant SRE is discussed first. At 832, system 48 determines whether the highest magnetometer magnitude exceeds first upper threshold value. If so, system 48 determines a “safe” status at 834, and may thus proceed to step 614 in state-based algorithm 600 (FIG. 17). If not, the method proceeds to 836, where system 48 determines whether the highest field strength magnitude is below a first lower threshold value (which is lower than the first upper threshold value). If so, system 48 determines a “danger” status at 838, and may thus proceed to step 618 in state-based algorithm 600 (FIG. 17). If not, system 48 determines a “marginal safety” status at 840, and may thus proceed to step 616 in state-based algorithm 600 (FIG. 17).

The situation in which stylet 44 is oriented or moving away from the VL blade or relevant SRE is now discussed. At 842, system 48 determines whether the highest magnetometer magnitude exceeds second upper threshold value. If so, system 48 determines a “safe” status at 844, and may thus proceed to step 614 in state-based algorithm 600 (FIG. 17). If not, the method proceeds to 846, where system 48 determines whether the highest field strength magnitude is below a second lower threshold value (which is lower than the second upper threshold value). If so, system 48 determines a “danger” status at 848, and may thus proceed to step 618 in state-based algorithm 600 (FIG. 17). If not, system 48 determines a “marginal safety” status at 850, and may thus proceed to step 616 in state-based algorithm 600 (FIG. 17).

In one embodiment, the second upper threshold value and second lower threshold value are each higher than the respective first upper threshold value and first lower threshold value, to thereby enforce more stringent proximity conditions for the second case, i.e., the case in which the stylet 44 is oriented or moving away from the VL blade or relevant SRE.

Determining Stylet Orientation (e.g., Pitch, Yaw, Etc.)

Once the location of the magnet(s) 46 has been determined, there are multiple possible ways to determine the angular orientation of the styletted ETT. For example, once the relative location of a magnet 46 at the distal tip of the stylet 44 is determined, the combination of the 3-axis magnetic field data from one magnetometer 24 and the known distance between the magnet 46 and magnetometer 24 can be used to determine a relative orientation of the magnet's polarity. As the magnetic polarity is fixed on the endotracheal tube (or stylet 44), the orientation of the entire styletted tube 40 can be determined. As discussed above, the stylet 44 may be placed in the internal lumen of the endotracheal tube 42, such that the location/orientation of the stylet 44 correlates to the location/orientation of the endotracheal tube 42.

An additional approach to calculate orientation is to use historical location measurements. A “best fit” line in 3D space can be created between the recent historical locations, which represents the recent direction of motion of the ETT 40. Assuming a rigid ETT, the direction of motion is equivalent to the orientation.

An additional approach is to use several magnets 46 with known spacing that are placed along the stylet 44. Each magnet 46 is detectable by distinct magnetometers 24. The system may determine vectors between the magnets 46, which may represent the orientation of the ETT 40.

FIG. 26 illustrates an example algorithm 860 for calculating stylet pitch/yaw data for a stylet including two or more magnets or magnetized regions 46, according to one embodiment. The two or more magnets or magnetized regions 46 may be spaced apart along the length of stylet 44. At 862, system 48 may read data from all available magnetometers 24. At 864, system 48 may determine a magnetic field strength of each magnetometer 24. At 866, system 48 may identify one or more magnetometers 24 with high field strengths relative to other magnetometers 24. At 868, system 48 may assign one or more magnetic hot spots (up to the number of magnets 46 on stylet 44).

At 870, system 48 may calculate a vector between predicted hotspot locations (if there are more than two hotspots, the vector may be calculated as a best fit line). The vector between hotspots may be equivalent to the orientation vector of stylet 44. As indicated at 872, assuming the VL handle is properly aligned relative to the patent anatomy, the vector of stylet 44 relative to the VL handle is equivalent to the pitch/yaw of the stylet 44 during insertion.

FIG. 27 illustrates an example algorithm 880 for calculating stylet pitch/yaw data for a stylet including a single magnet or magnetized region 46, according to one embodiment. At 882, system 48 may read data from all available magnetometers 24. At 884, system 48 may determine a magnetic field strength of each magnetometer 24. At 886, system 48 may identify three or more magnetometers 24 with high field strengths relative to other magnetometers 24.

At 888, based on the magnetometer field strength values, system 48 may determine a distance of stylet magnet 46 from each of the three or more high field strength magnetometers 24. At 890, based on the known locations of the three or more high field strength magnetometers and their respective field strengths, system 48 may calculate a vector of the magnetic north-south pole that best satisfies the constraints. As indicated at 892, the north-south pole of magnet 46 may be equivalent to the orientation vector of stylet 44. Further, as indicated at 894, assuming the VL handle is properly aligned relative to the patent anatomy, the vector of stylet 44 relative to the VL handle is equivalent to the pitch/yaw of the stylet 44 during insertion.

Intubation Guidance without Magnetometer Calibration

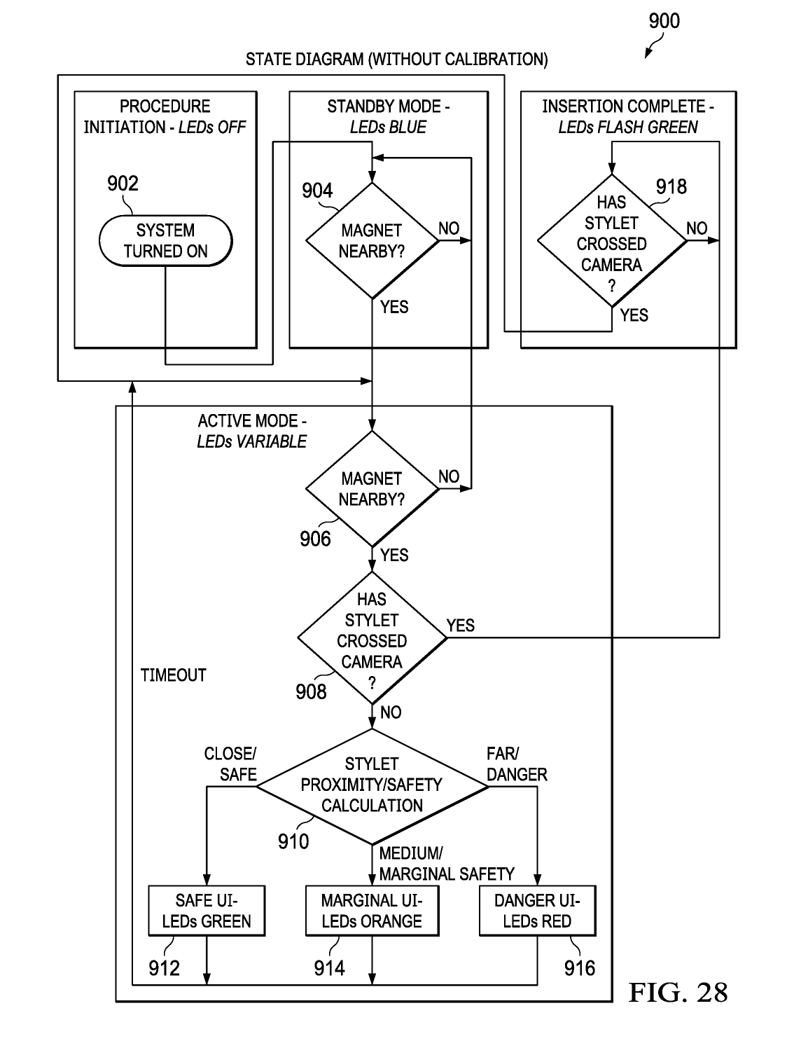

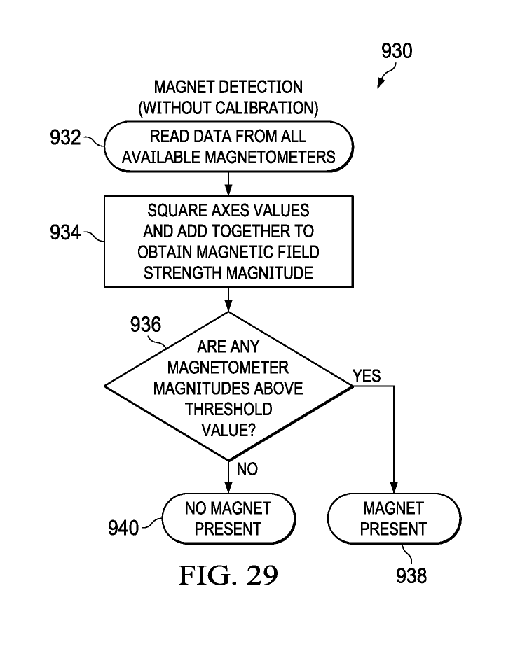

FIG. 28 illustrates an example state-based algorithm 900 for providing guidance-based facilitation of an intubation procedure using a magnet-based stylet guidance system 48, according to an example embodiment. Algorithm 900 is an alternative to algorithm 600 shown in FIG. 17, for a case without magnetometer calibration. Thus, algorithm 900 is similar to algorithm 600, but omitting the magnetometer calibration step (step 604 of algorithm 600). As a result of omitting the magnetometer calibration step, algorithm 900 shown in FIG. 28 utilizes a modified magnet detection algorithm 930 shown in FIG. 29 in place of magnet detection algorithm 650 shown in FIG. 19.

FIG. 29 illustrates a magnet detection algorithm 930 for detecting whether a magnet 46 is nearby, for an embodiment without magnetometer calibration, according to one embodiment. At 932, system 48 may read data from all available magnetometers 24. At 934, system 48 may square and sum the calibration axes values for each magnetometer to calculate a magnetic strength magnitude for each magnetometer. At 936, system 48 may determine whether any magnetometer magnitudes are above a defined threshold value. If at least one magnetometer magnitude exceeds the threshold value, the system 48 determines that a magnet is nearby at 938. Otherwise, if none of the magnetometer magnitudes exceed the threshold value, the system 48 determines that no magnet is nearby at 940.

Airway Anatomy Mapping, e.g., Via Acoustic and/or Electromagnetic Interrogation

In some embodiments, an intubation guidance system (e.g., system 48 discussed above) may include additional sensors configured to map out a patient's airway. Such airway mapping sensors may be incorporated into the VL handle, for example. For example, the system may include one or more airway mapping sensors arranged in the VL handle and configured to create a 3D map of the patient's airway in real-time. The system may use the ultrasound mapping of the airway to measure distances to specified structures (e.g., the pharyngeal wall, tonsils, trachea, esophagus, etc.). As discussed above, the system may use signals from 3D magnetometers to determine the distance between the stylet and VL device. The system may combine these two sensing modalities to ensure that stylet 44 is not too close or too far from specified airway structures. For example, in embodiments in which the system uses stylet-VL distance thresholds regarding the detected distance between the stylet and VL device (e.g., to trigger defined feedback via guidance indictors 30), the system may set or adjust these distance thresholds based on the patient's unique anatomy, as determined using the airway mapping sensors. The system may include any suitable type of sensors that can be used for airway mapping, e.g., ultrasound, optical, radiofrequency, radar, or laser. These sensors can be used alone or in combination to generate an accurate 3D model of the airway in real-time.

Additionally, the system may utilize airway mapping sensors to identify the location of the trachea/vocal cords. The system may use this information to help guide styletted ETT 40 towards the trachea, which may be particularly useful if the trachea/vocal cords are not visible (e.g., visually occluded by blood, fluid, secretions, etc.). For example, the system may generate an augmented reality view via video monitor 22, which may display virtual representations of selected airway features (e.g., the trachea/vocal cords) and/or medical devices (e.g., ETT 40 and/or VL blade 16) superimposed over or otherwise combined with the video images captured by VL camera 20. Example embodiments of such augmented reality system are discussed below with reference to FIGS. 31-33.

Some embodiments may include various combinations of one or more types of sensors, e.g., one or more accelerometers, Hall sensors, 3-axis Hall sensors, magnetometers, bioimpedance sensors, acoustic sensors, ultrasonic sensors, radar, lidar, sonar, and/or other types of sensors for the purpose of determining stylet positioning information and/or anatomical mapping of the airway.

In some embodiments, intubation guidance system may be integrated in a video laryngoscopy system during manufacturing. In other embodiment, an intubation guidance system may be designed as an “after-market” system that can be used in conjunction with an existing video laryngoscopy system. For example, as discussed below, some embodiments provide an intubation guidance system including (a) sensors for detecting the location and/or orientation of a styletted endotracheal tube, (b) processing and memory devices for executing algorithm(s) to analyze sensor data from such sensors, and (c) at least one display (e.g., LEDs) for indicating the location and/or orientation of the styletted endotracheal tube, provided in one or more units that can attach to a video laryngoscopy handle and/or fiber optic cable and/or can be inserted or arranged within the disposable blade of a video laryngoscopy system.

Some embodiments may be configured to perform real-time 3D mapping of the airway or other anatomical structures, e.g., to detect or avoid a possible injury to the patient. The system may be configured to determine that the 3D mapping of the throat is anatomically atypical or irregular, and in response trigger a warning to the operator to pay special attention to the case. One example embodiment may include a ring of sensors arranged in one plane of the handheld VL device. As the VL device is inserted, data from accelerometers, gyroscopes and/or other sensors can be used to detect the location of the VL device along the curve of throat. The ring of sensors may take “cross-section” measurements of the throat at a defined frequency. The system may then create a 3D model by combining the cross-sections along the curve of the throat. Some embodiment may utilize ultrasonic and/or RF techniques for such mapping, e.g., due to the presence of fluids in the mouth. The system may utilize any suitable 3D scanning technologies known by those skilled in the art.

In some embodiments, an ultrasonic transducer may be embedded within, or otherwise associated with, the electronic apparatus of the video laryngoscope. In some embodiments, the ultrasonic transducer is integrated near the distal tip of the video laryngoscope. The ultrasonic transducer produces acoustic or ultrasonic waves that reflect off nearby tissues. An ultrasonic map of the airway anatomy can be produced based on the pattern of the reflected waves. The density of tissues and their proximity to the ultrasonic transceiver can be determined by the ultrasonic pattern generated.

Ultrasound technology has provided a reliable and effective way to interrogate airway anatomy. Structures within the airway produce unique ultrasonic signatures that can be readily identified. For example, the vocal cords will appear as triangular hypoechoic structures outlined by hyperechoic structures (vocal ligaments). Cartilaginous structures (such as the trachea) will appear as hypoechoic regions with a characteristic bright interface that represents the intraluminal surface (i.e. tissue-air boundary).

As discussed previously in this application, ultrasonic examination of the airway anatomy can be performed in a transverse plane, through the anterior surface of the neck. However, with an ultrasonic transceiver integrated into the video laryngoscope, the ultrasonic interrogation approach is intraoral. Because airway structures are filled with air, the quality of ultrasonic imaging can be poor. Air has a high acoustic impedance and does not transmit ultrasound signals well. Ideally, the ultrasonic transducer will be in direct contact with tissue to produce the best quality image. With the intraoral approach, the ultrasonic transducer may not be in direct contact with tissue.

Ultrasonic range finding, or sonar, can also be used to interrogate the airway anatomy. With this method, an ultrasonic pulse is generated in a known direction. When the pulse hits structures of varying density, the pulse will be reflected back to the transmitter as an echo in a characteristic manner. It is possible to determine the distance away from structures of varying density by measuring the difference in time between the pulse being generated and the echo being received.

Similar to sonar, the airway can also be interrogated using radar or lidar. Radar and lidar are similar to sonar, but instead they emit an electromagnetic pulse, instead of an acoustic pulse. Radar utilizes radio waves while lidar utilizes light (or laser) waves. Radio and lidar may be perform better in open air, as they are not negatively impacted by the high acoustic impedance of air.

Ultrasound, sonar, radar, and lidar can be used individually or in combination to define the airway anatomy in real time. This information can be combined with optical information produced by a video laryngoscope. In such a manner, a comprehensive assessment of a patient's airway can be provided that is less sensitive to “blind spots” or undesirable visual obstructions (i.e. blood, fluid, vomit, etc. obscuring camera). Ultrasound, sonar, radar, and lidar can also help define the airway anatomy in multiple dimensions simultaneously, which allows the airway to be monitored even outside of the camera's field of view.

Comprehensive, multi-modality airway visualization/interrogation provides a number of potential benefits beyond optical-only visualization:

### Example Benefit #1: Minimize/Prevent Airway Trauma

As described elsewhere in this application, the video laryngoscope's “blind spot” is a region where instruments/equipment (i.e. endotracheal tube) need to traverse without visual guidance. Given the lack of visual guidance in the “blind spot”, it is possible for airway structures, such as the tonsils, to be damaged by instruments/equipment. By providing information obtained from acoustic or electromagnetic airway interrogation, the blind spot can be effectively eliminated. For example, while the camera of a video laryngoscope may be focused on the primary target (vocal cords, trachea), acoustic and electromagnetic sensing modalities can be used to monitor the peripheral anatomy. When an instrument is then placed into the airway (such as an endotracheal tube), which can be identified based on its magnetic signature, the system can ensure that the magnetically tagged instrument stays sufficiently far away from airway tissues, such as the tonsils. If the instrument gets too close to airway tissues, this information can be provided to the user via acoustic feedback (i.e. beeps) or visual feedback (i.e. virtual representation of the spatial relationship between the airway anatomy and the instrument), or haptic feedback (i.e. vibration).

### Example Benefit #2: Facilitate Identification of Airway Targets

As described elsewhere herein, there are situations when the optical image produced by a video laryngoscope can be obscured (i.e. blood, vomit, etc.). When an obscured optical image, it can be difficult to visually locate critical airway structures, such as the vocal cords. By leveraging non-optical based imaging techniques (i.e. ultrasound, sonar, lidar, radar), airway structures can be identified to facilitate endotracheal intubation despite poor optics. For example, lidar can be used to identify anatomic structures consistent with the trachea and this information can be visually presented as an overlay on the optical image.

Danger Avoidance

Some embodiments may use magnetometer(s) 24 and magnet(s) 46 to help actively avoid dangerous areas. For example, one embodiment includes an electromagnetic or multiple electro-magnets included within the VL handle, and the system is configured to an “active feedback.” If the system determines that the stylet magnet 46 has strayed too far from the VL handle, the system could activate the electromagnet(s) to provide resistance before a dangerous event occurs. This electromagnetic system could also be used continuously as a form of guidance without a mechanical track.

The system may dynamically modulate the strength of the electromagnet(s) to prevent stylet 44 from magnetically “sticking” to the VL handle. Once the stylet 44 is sufficiently close to the VL handle, the system may deactivate the VL electromagnet(s). As the stylet enters a more dangerous position (e.g., further from the VL handle), the system may activate and increase/decrease the strength of the VL electromagnet(s) as a function of stylet distance. Once the stylet passes the camera plane in the insertion direction, the system may turn off the VL electromagnet(s) to allow completion of the intubation procedure.

One embodiment may also use this electromagnetic guidance or “encouragement” the of the stylet positioning when the airway anatomy is more accurately mapped out via any of the sensing modalities or techniques disclosed herein (e.g., 3D airway mapping using ultrasound, RF, laser, etc.). If the system determines that the stylet is nearing a dangerous structure or position, the system may activate the VL electromagnet(s) to “encourage” the stylet toward a direction to thereby avoid danger.

Neck Sensor Apparatus

As discussed above, some embodiments of the intubation guidance system may include or operate in cooperation with an external neck sensor apparatus placed on the outside surface of a patient's neck and configured to determine the location of the trachea (or other anatomical features), which may be displayed to the proceduralist via a virtual display to facilitate a non-optical intubation. For example, some embodiments may incorporate or operate in cooperation with any of the neck apparatuses or techniques disclosed in any of the Neck Sensor Applications listed above.

The neck sensor apparatus may be configured to assess the airway by identifying physiological information regarding the patient, e.g., the trachea depth or location. Some embodiments may also be able to determine the patient's neck circumference, which has a known relationship to tracheal depth, based on signals from a plurality of gyroscopes/accelerometers (i.e., angle gauge) positioned in an array around at least a portion of the patient's neck. The gyroscopes/accelerometers may provide data to a microcontroller or microprocessor which can then calculate neck circumference algorithmically. Alternatively, neck circumference could be measured using an automated tape measure that optically determines exposed tape length via a color pattern on the tape. Alternatively, or in conjunction, ultrasonic transceivers can be used to measure the depth and location of the trachea. In this embodiment, the depth of the trachea will be determined directly by measuring the acoustic impedance between pre-tracheal soft tissue and tracheal air. The sensing device can utilize a software application for displaying anatomic data on a video laryngoscope screen or other display device, such as a smartphone, computer, or the like. Given that certain anatomic relationships and features (i.e. large neck circumference) correlate with difficulty of intubation, the system may provide an objective assessment of the likelihood of a difficult intubation and may further provide, depending on the embodiment, both visual and audible feedback regarding the location of an medical device relative to the trachea.

Some embodiments provide a comprehensive airway management system includes a neck apparatus for analyzing the airway anatomy, in combination with a system for determining the location of a styletted endotracheal tube relative to a video laryngoscope. This combination may allow for improved guidance of the styletted endotracheal tube into the trachea, thus increasing the chances of safe intubation and reducing the possibility of a traumatic intubation.

In some embodiments, an electromagnet is incorporated into the neck apparatus, such that the strength of the magnetic field provided by the electromagnet can be modulated. In this manner, the magnetic intubation stylet can be physically guided into the trachea using magnetic attraction forces. The neck apparatus can push or pull the stylet based on the stylet's location relative to the trachea. The strength of magnetic attractive forces applied may vary based on the location of the stylet relative to the trachea.

The neck apparatus may include a sensor-based sensing apparatus placed on, near, or around the patient's neck. The neck apparatus can be placed in a standard orientation with respect to the patient's neck. For example, the neck apparatus may be placed in the midline of the patient's neck, directly over the thyroid cartilage. The orientation of the neck sensor apparatus with respect to the patient can be defined or determined. In some embodiments, the neck apparatus includes markings, or a characteristic shape, or another means for indicating how to properly orient the neck sensor apparatus with respect to the patient. In some embodiments, the orientation of the neck sensor apparatus with respect to the patient is automatically determined. In some embodiments, the neck sensor apparatus can automatically determine the depth and location of the trachea and other anatomic landmarks relative to the device regardless of device orientation with respect to the patient.

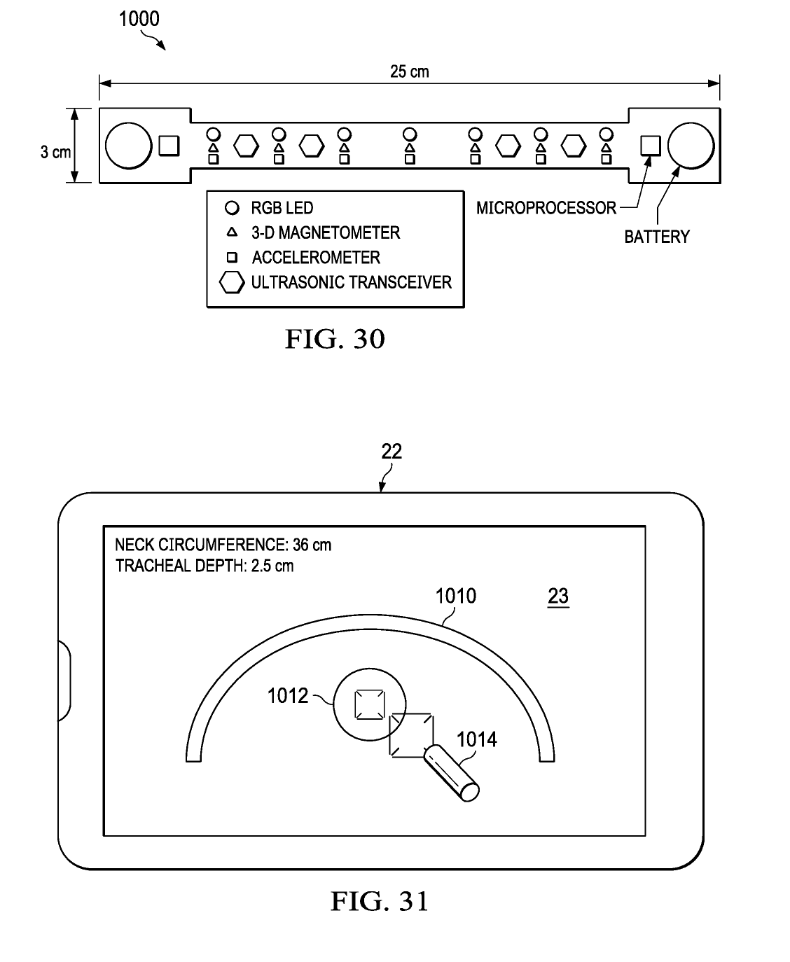

FIG. 30 illustrates an example neck sensor apparatus 1000, e.g., as disclosed in the Neck Sensor Applications, which may be included in certain embodiments of the present invention. The neck sensor apparatus may be configured to be placed substantially on or around a patient's neck, and may utilize a variety of sensor modalities to determine the location of the patient's trachea.

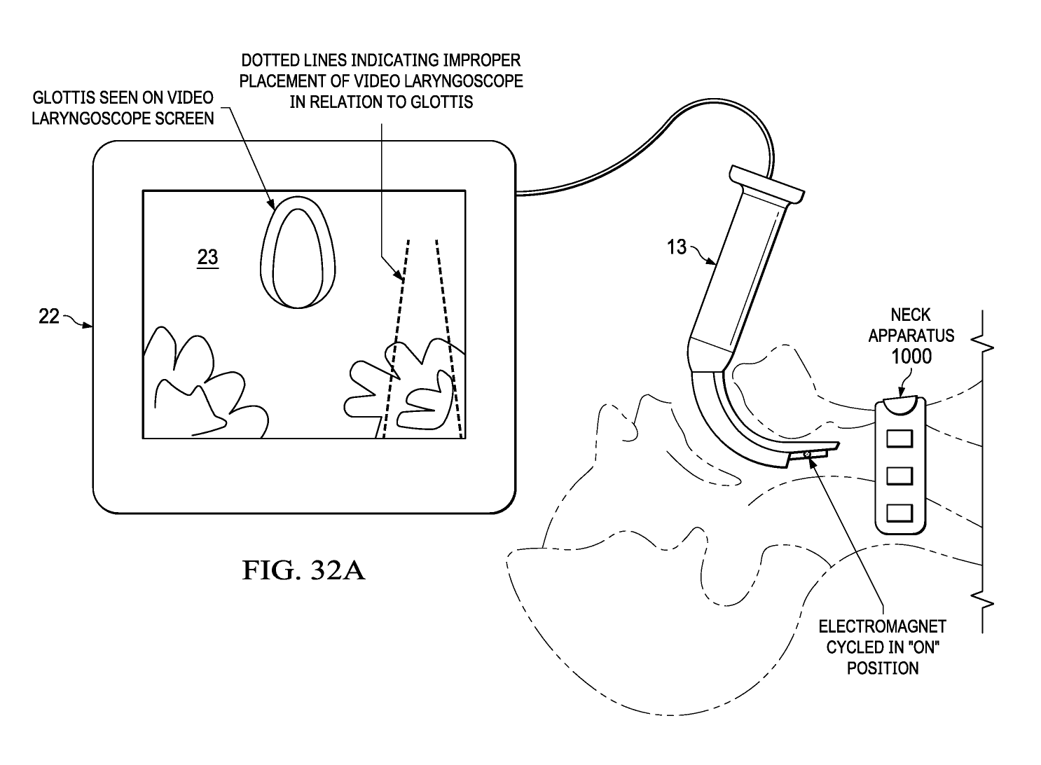

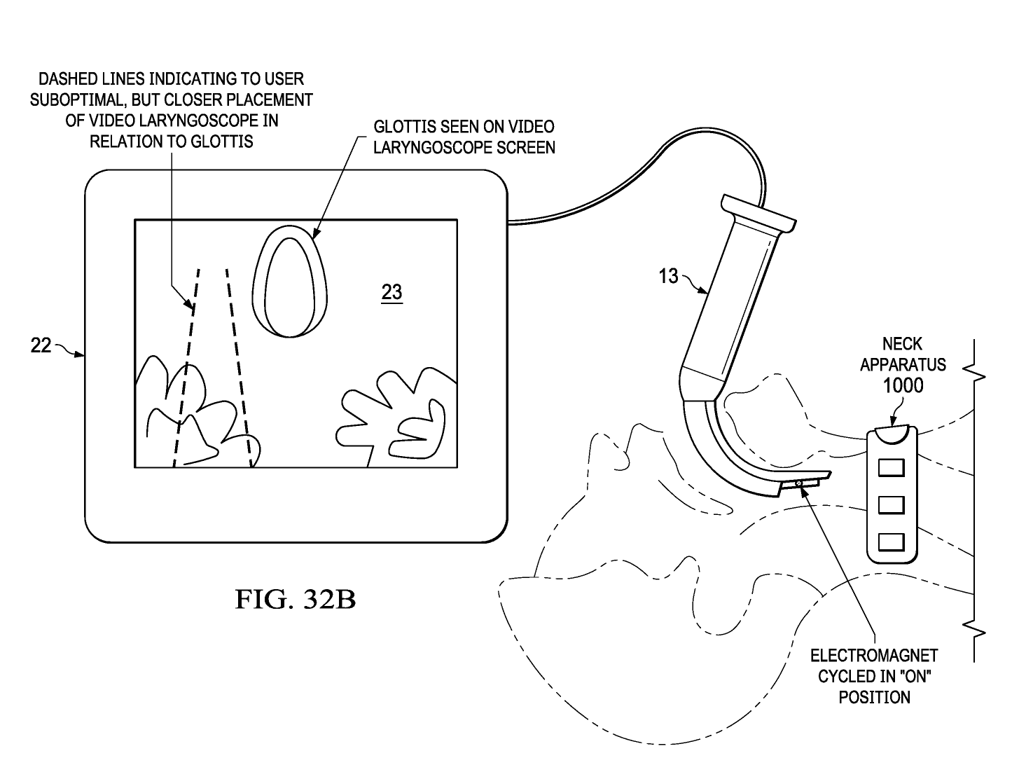

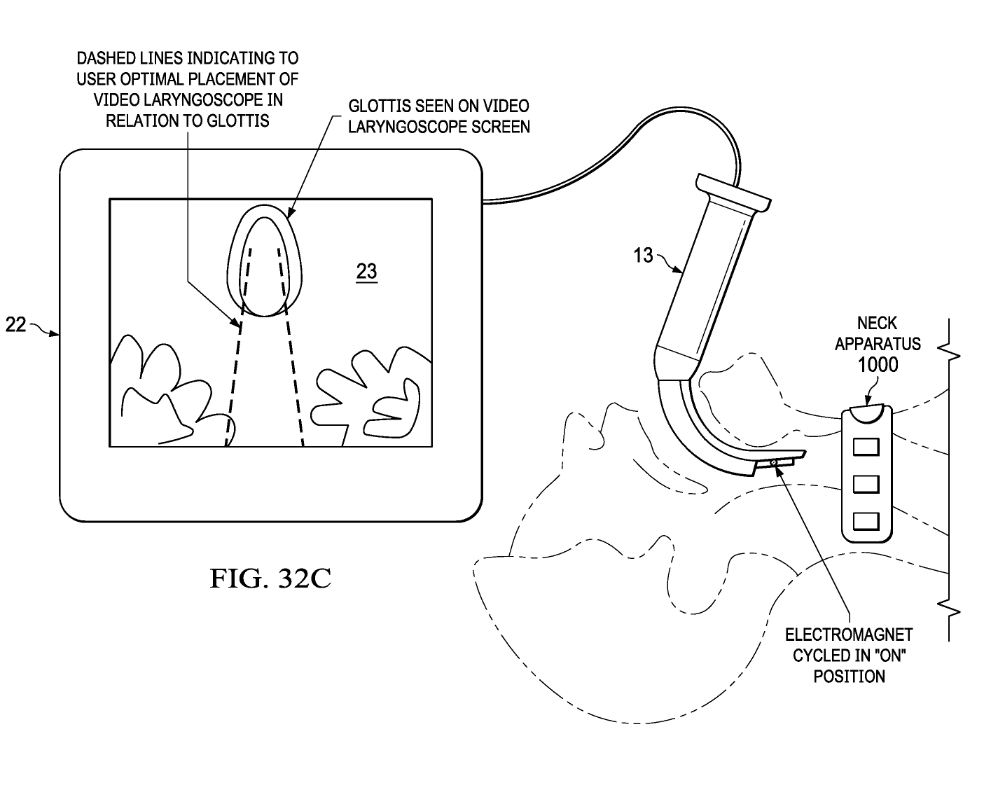

FIGS. 32A-32C illustrate a system including a video laryngoscope 13, neck sensor 1000, and a VL monitor 22 with video screen 23, according to an example embodiment. In particular, FIGS. 32A-32C illustrate the arrangement and interaction of neck sensor apparatus 1000 arranged on the neck and video laryngoscope 13 positioned in a patient's airway, and an example display on screen 23 that indicates virtual representations of the glottis and the video laryngoscope 13, as detected by neck sensor apparatus 1000, overlaid on a video image captured by the VL camera. In some embodiments, the system also includes a magnet-based stylet guidance system 48, e.g., as discussed above, which may be integrated with or distinct from video laryngoscope 13. In such embodiments, the stylet guidance system 48 may detect and monitor stylet position information, and display a virtual representation of the styletted ETT on display 23, e.g., based on the spatial relationship between the styletted ETT and video laryngoscope 13 as determined by the magnet-based stylet guidance system 48.

Referring to FIG. 30, the example neck sensor apparatus 1000 may include a substrate that supports or houses a controller or processor together with other appropriate logic as will be understood by those of ordinary skill in the art. In some embodiments, the substrate includes a tab that may provide an asymmetry to the substrate to help facilitate proper orientation of the substrate with respect to the patient, and can be sized to permit comfortable placement of the substrate on the neck. The neck apparatus may include one or more sensors that are responsive to a magnetic field and provide their outputs to the controller. Examples of possible sensors include Hall effect sensors, 3-axis Hall sensors, magnetometers, electronic compass, reed switches, fluxgate magnetometers, magnetoresistance-based sensors, or other magnetic field or proximity sensors. Throughout this disclosure, the term “magnetometer” is used for clarity of disclosure, but is intended to encompass any suitable magnetic or electric field sensor. In some embodiments, the sensors may include accelerometers for assisting in the determination of neck circumference, as discussed in greater detail below. The accelerometers and magnetic sensors may or may not be implemented in the same electronic chip.

In one embodiment, at least a portion of the magnetic neck apparatus extends directly over the trachea, when arranged on the patient's neck, e.g., as shown in FIGS. 32A-32C. The trachea is located anterior to the esophagus, with the vocal cords located proximate to the entry to the trachea. In other embodiments, the neck apparatus can be placed over the neck in any orientation and the orientation of the apparatus relative to the patient is automatically determined. In this manner, the sensitivity and specificity of detecting endotracheal placement of the magnetic intubation stylet can be increased.

The curvature of a patient's neck and the degree of soft tissue compliance can vary widely among patients. In some embodiments, the neck apparatus is sufficiently flexible to bend and conform to the curved surface of the patient's neck. The apparatus may have a flexible substrate, such as a polyimide conductive fabric, or any other suitable material. In some embodiments, the neck apparatus may be wrapped completely around the neck or around a portion of the neck. In other embodiments, the neck apparatus may not be in direct contact with the patient, but instead is remotely associated with the patient in a known orientation/location relative to the patient. In other embodiments, the substrate may comprise a material having memory, such that once positioned on a patient's neck, the shape of the neck is preserved at least somewhat by the memory characteristic of the material, thus helping to ensure proper placement and retention on the anterior surface of the next. In some embodiments the neck apparatus may contain a sensing unit or system to determine the extent to which the apparatus is flexed, bent, wrapped around, or is otherwise associated with the patient's neck or other tissues. Furthermore, in some embodiments the neck apparatus may include a system or unit for determining the degree of contact with a subject's skin, such as by capacitive or thermal sensing or other means. In some embodiments, the neck apparatus may provide user feedback to indicate whether the apparatus is making sufficient contact with the patient, or the neck apparatus can automatically calibrate itself based on the degree to which the neck apparatus is contacting the patient.

In some embodiments, the output of the sensor(s) is related to the magnitude and direction of an externally applied magnetic field. In some embodiments, the apparatus includes an array of magnetometers. The magnetometers may be arranged in different relative orientations, which may be predefined or determined. In some embodiments, the neck apparatus has a curved structure or is flexible. When arranged along an arc or a curve, a three-dimensional magnetic sensor array is created. By orientating the magnetometers in a partially or completely circumferential pattern around the target, the relative location, orientation, and trajectory of an externally applied magnet can be accurately determined. Having a magnet with a known orientation can help users align the short-axis of the video laryngoscope blade with the long-axis of the trachea in order to facilitate endotracheal intubation. In some embodiments, particularly if single-axis magnetometers are used, some magnetometers may be placed orthogonally to others.

In one embodiment, the neck sensor apparatus contains an array of LEDs. The brightness or color of each LED can indicate the location of the video laryngoscope tip or intubation stylet relative to the trachea. For example, if the direction of the video laryngoscope trajectory is lateral to the trachea, the LEDs corresponding to the lateral side of the apparatus can be illuminated. As the video laryngoscope tip moves in the direction and trajectory of the trachea (midline structure), the central LEDs may begin to illuminate while the lateral LEDs get progressively dimmer or change color. The brightness of each LED can also indicate the depth of the video laryngoscope tip or styletted endotracheal tube with respect to the trachea. For example, as the magnet is positioned deeper to the trachea (more posterior), the LED intensity can decrease. In addition to manipulating the brightness, the color of each LED can also be altered to provide specific feedback to the operator. The LEDs may also flash at a fixed or variable rate to provide additional visual feedback. Any pattern of visual (or auditory) feedback can be employed to facilitate endotracheal intubation, as long as the visual/auditory cues properly represent the relative location, orientation, and trajectory of the video laryngoscope tip or intubation stylet with respect to the trachea and can be understood by a user, for example, emergency personnel or an anesthesiologist.

In some embodiments, successful insertion of the magnetic intubation stylet into the trachea is represented by a characteristic light pattern, an audio chime, or a haptic pulse on the video laryngoscope handle. When the location of the magnetic intubation stylet is determined to be within the boundaries of the trachea, in indication of successful intubation can be provided to the care provider.

As noted above, in some embodiments, the brightness of color of one or more LEDs can indicate the probability that either the tip of the magnetic video laryngoscope blade is aligning with the trachea or that the magnetic stylet has been successfully inserted into the trachea. However, if the stylet is inadvertently placed into the esophagus, the LEDs may not illuminate, or may be dim, or may change a characteristic color, depending on the embodiment. Any pattern of visual or auditory feedback that provides meaningful information to the care provider can be employed to indicate the probability that the intubating stylet is in the correct location.

In some embodiments, data from the neck sensor apparatus is communicated through a user-interface that displays a visual representation of the direction of the magnetic video laryngoscope tip to anterior neck topography, the trachea or other anatomic landmarks. Similarly, once the video laryngoscope is properly placed, the magnet within the video laryngoscope tip can be cycled “OFF” to allow for the magnetic intubation stylet to be located in relation to both the trachea as well as the video laryngoscope. In this manner, a virtual 2-D or 3-D image of the intubation procedure can be displayed to a user. In some embodiments, the user-interface is communicated wirelessly to a remote display, such as a computer terminal or mobile device. In other embodiments, a wired communication link exists between the neck apparatus and the user-interface display. The virtual 2-D or 3-D image of the intubation procedure can also be overlaid onto the existing video laryngoscope monitor.

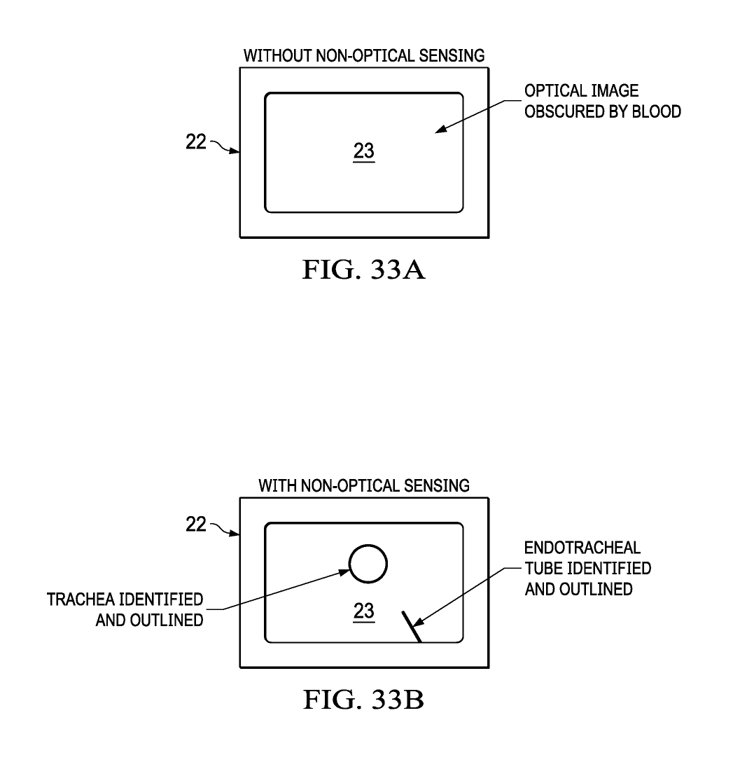

FIGS. 33A and 33B illustrate an example VL video display for a VL system with and without non-optical sensing, for a situation in which the VL camera is blocked/occluded, according to an example embodiment.

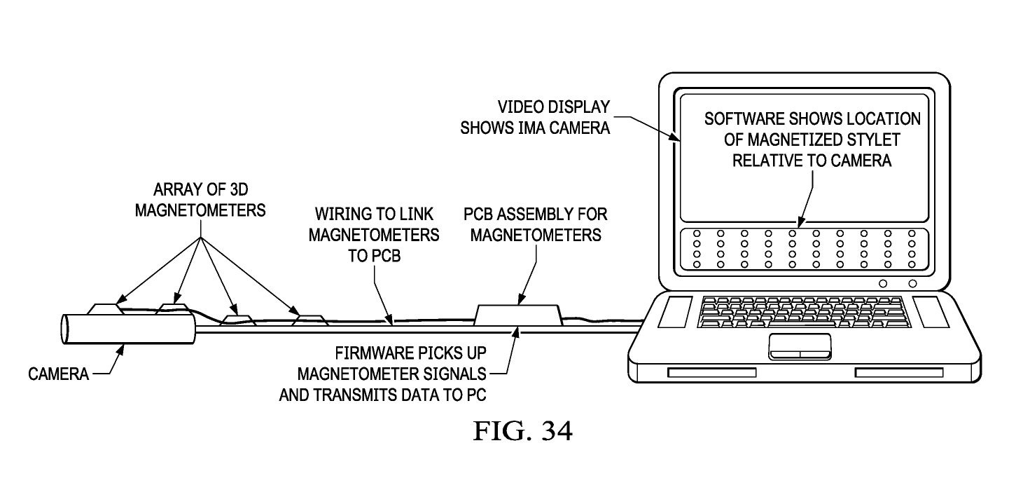

FIG. 34 illustrates an example system for collecting and analyzing signals generated by an array of 3D magnetometers integrated into a video laryngoscope, e.g., to determine the location or orientation of an endotracheal tube relative to the laryngoscope camera, and displaying the determined endotracheal tube location or orientation via a video display, according to an example embodiment.

Intubation Guidance System with Neck Sensor and a Cycled Electromagnet

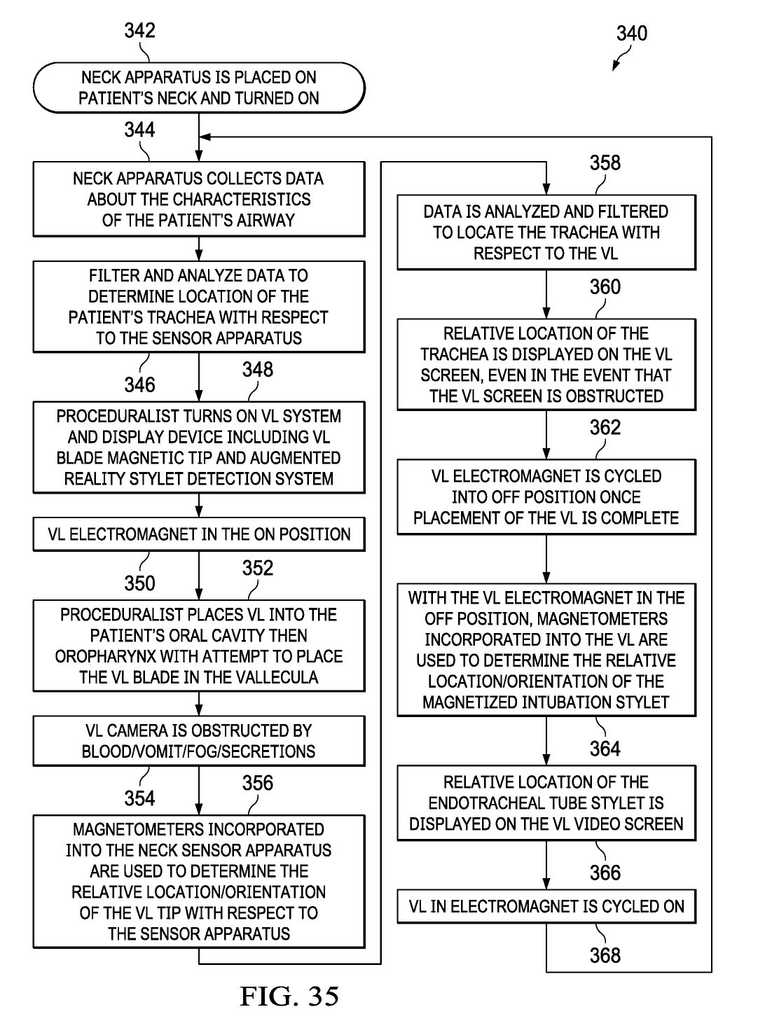

FIG. 35 illustrates an example clinical algorithm 340 in the setting of intubation when the VL camera is obstructed, but with guidance from the neck sensor apparatus, according to an example embodiment. In this embodiment, an embedded electromagnet at the tip of the laryngoscope is switched between an ON state and an OFF state to provide. By cycling the electromagnet ON and OFF, and when used in conjunction with the neck sensor apparatus 1000, the system may be able to assess both the relationship of the video laryngoscope to the trachea as well as the position of the styletted endotracheal tube to the video laryngoscope, as discussed below.

Some embodiments provide a video laryngoscope with a plurality of magnetic field sensors distributed substantially along the axis of the laryngoscope, as well as an embedded electromagnet positioned at the tip of the video laryngoscope blade. These sensors may be arranged circumferentially. The arranged array of magnetic field sensors may extend substantially from the base to the tip of laryngoscope. The embedded electromagnet at the tip of the laryngoscope may be switched between an ON state and an OFF state. When the electromagnet is switched ON, the magnetic field signature can be detected by an external magnetic field sensor, such as those in the neck sensor apparatus shown in FIG. 30, for example. When the electromagnet is switched OFF, the only magnetic field signature in the environment is from a permanent magnet, such as from a magnetized endotracheal tube stylet. In such a fashion, it is possible to determine the location/orientation of the video laryngoscope with respect to the neck sensor apparatus. Given that the neck sensor apparatus is designed to determine the relative location of the trachea and other anatomic structures, it is possible to transitively determine the location/orientation of the video laryngoscope with respect to the trachea and other anatomic structures. Furthermore, by using the array of magnetometers in the video laryngoscope, it is possible to determine the relative location of a magnetized ETT stylet with respect to the video laryngoscope and anatomic structures.

In some embodiments, the video laryngoscope or the video laryngoscope disposable blade may include a plurality of magnetometers throughout the length of the instrument. The array of magnetometers may span from the handle of the instrument to the tip of the blade or in some embodiments to the level of the video laryngoscope camera. The magnetometers may be embedded directly into the video laryngoscope, or alternatively, built into a disposable blade of the video laryngoscope. The plurality of magnetometers embedded within the laryngoscope or laryngoscope blade can be designed around any previously existing laryngoscope blade or VL system. The arrangement of the magnetometers may be designed in any suitable manner and configuration, such that a magnet is detectable from any angle of the laryngoscope, from handle to blade tip, for example. Those of ordinary skill in the art will recognize that there are many possible ways of incorporating a plurality of magnetometers into a non-disposable laryngoscope or disposable laryngoscope blade. Any device or apparatus that is intended to be used as an instrument for intubation or a method of visualizing the airway can be embedded with a plurality of magnetometers to enable detection of a magnetized medical device, such as an endotracheal tube stylet.

The magnetometers that may be incorporated into the laryngoscope may draw power from the video laryngoscope. Additionally, an electromagnet may be incorporated into the distal tip of the video laryngoscope and positioned in a fixed and known orientation with respect to the video laryngoscope, such as at the distal tip of the camera. The electromagnet may be rapidly cycled “ON” and “OFF” and may be powered by the video laryngoscope battery. By cycling the electromagnet ON and OFF, and when used in conjunction with the neck sensor apparatus, the system may be able to assess both the relationship of the video laryngoscope to the trachea as well as the position of the styletted endotracheal tube to the video laryngoscope, e.g., as illustrated in example algorithm 340 shown in FIG. 35.

In the case of an intubation that may be difficult, or wherein it is predicted that the VL camera may become obstructed (i.e. blood/fluid), prior to the introduction video laryngoscope or magnetic stylet into the person, the neck sensor apparatus can be placed on the neck as described under the section “Neck Apparatus” to perform a pre-intubation airway assessment. As detailed in algorithm 340 shown in FIG. 35, the video laryngoscope may be introduced in the standard fashion, with the magnetic tip of the video laryngoscope cycled in the “ON” state. When the distal tip electromagnet is in the “ON” state, the tip of the video laryngoscope can be detected by the neck sensor apparatus's magnetic sensors. Once appropriate positioning of the video laryngoscope is confirmed, the video laryngoscope electromagnet can be cycled to the “OFF” state. While in the “OFF” state, the magnetic styletted endotracheal tube can be introduced to the system. In some embodiments, the magnetic stylet provides the only external magnetic field and can be detected by both the video laryngoscope with its embedded array of magnetometers, as well as by magnetic sensors in the neck apparatus. Therefore, the system may continually detect and indicate to the proceduralist (e.g., via a display means) the relationship of the magnetic endotracheal stylet relative to the video laryngoscope, as well as the relationship of the magnetic endotracheal stylet to the trachea.

The components of the system described herein can be used in combination or separately. In some implementations, the neck sensor apparatus is not used. In this case, the video laryngoscope (or standard laryngoscope or other intubation device) uses an array of magnetometers to detect the relative location, orientation, or velocity of a magnetized endotracheal tube or endotracheal tube stylet.

Once the magnetic endotracheal tube stylet is detected by the video laryngoscope's embedded array of magnetometers, the relationship of the magnetic stylet to the blade of the laryngoscope may be indicated via a user-interface that displays a visual representation of the magnetic intubation stylet superimposed on the video laryngoscope monitor, or alternatively, on a screen adjacent to the VL monitor. In this manner, a virtual 2-D or 3-D image of the intubation procedure may be displayed. In some embodiments, the relationship between the magnetic stylet to the blade of the laryngoscope may be indicated via a separate display device from the video laryngoscope monitor. This secondary display may contain stylet coordinate information. In some embodiments, the user-interface is communicated wirelessly to a remote display, such as a computer terminal or mobile device. In other embodiments, a wired communication link is provided between the neck sensor apparatus or the video laryngoscope's magnetic sensors and the user-interface display. In still other embodiments, the user interface is provided locally on the video laryngoscope itself and communicated via a wired or wireless communication means to a remote display.

In some embodiments, the video laryngoscope will also include an embedded electromagnet at the level of the video laryngoscope camera. The video laryngoscope may be placed in the oral cavity and then the oropharynx in standard fashion for endotracheal intubation. During placement of the laryngoscope blade, the embedded electromagnet may be cycled to the “ON” state, such that the neck apparatus can detect the tip (or other known component) of the video laryngoscope blade, or in some embodiments, the disposable tip of the laryngoscope blade. According to the two-curve theory of intubation, successful laryngoscopy and tracheal intubation requires alignment of the oropharyngeal curve as well as the pharyngo-glottal-tracheal curve with the tangent point being the laryngeal vestibule axis. The neck sensor apparatus may detect the magnetic laryngoscope blade tip, thus helping the proceduralist align along the pharyngo-glottal-tracheal curve. The user interface may provide a virtual 2-D or 3-D position of the trachea in relation to the laryngoscope tip. Virtual targeting boxes or other visual indicators may be displayed to visually represent the distance and direction of the trachea in relation to the video laryngoscope tip. For example, as the magnet approaches the direction of the trachea, the guidance overlay may change in color or provide alternative audio, or visual, or haptic signals to indicate to the proceduralist whether the video laryngoscope blade is placed in a proper trajectory to effectively facilitate endotracheal intubation.

As discussed above, the array of magnetometers embedded within the laryngoscope may detect the presence of an endotracheal tubes stylet's embedded magnet when the electromagnet at the tip of the video laryngoscope is cycled “OFF”. Given that the electromagnetic will produce a strong magnetic field, it is necessary to turn this magnet OFF in order to detect smaller external magnetic fields in the local environment, such as those produced by a small magnetic embedded into an endotracheal tube stylet. In some embodiments, when the video laryngoscope electromagnet in the “OFF” state, the only magnetic field signature in the environment will be emitted from the magnetic endotracheal tube stylet. The array of magnetometers embedded in the video laryngoscope may allow the location of the magnetic stylet relative to the video laryngoscope to be continuously detected and displayed to the proceduralist via a suitable user interface, directly on the VL monitor, or alternatively on an adjunct display, as discussed above.

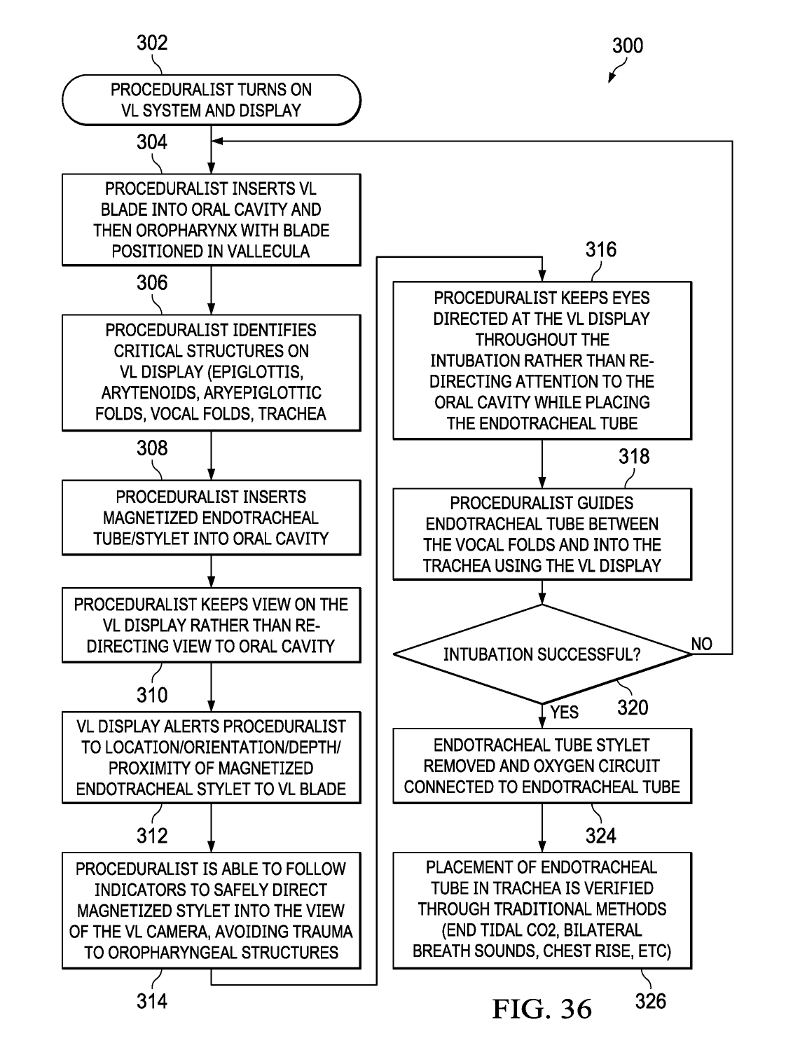

FIG. 36 illustrates an example clinical algorithm 300 in the setting of intubation without neck sensor apparatus, where the VL camera is not obstructed, using magnetic guidance system for localizing the ETS with respect to the VL in order to avoid palate or oropharynx trauma, according to an example embodiment.

Non-Optical Applications

An additional application of the present invention involves the ability to determine the location (e.g., 3-dimensional location), orientation, or velocity of an instrument, needle, or other device relative to a non-optical imaging device, such as an infrared camera, or x-ray generator. In such embodiments, a plurality of magnetic sensors is arranged in a fixed and known orientation with respect to an infrared camera, ultrasound, or x-ray generator. A permanent magnet or electromagnet may be incorporated into any instrument designed to be used in conjunction with the non-optical imaging device. In this example application, the non-optical imaging device may be capable of determining the 3-dimensional location of a nearby instrument, such as a needle, even before the needle is in view by non-optical means. Such a system may allow the correlation of the location of the instrument relative to the location of an anatomic structure, as identified by the non-optical imaging device.

In addition to the airway management techniques discussed above, certain embodiments or concepts disclosed herein may be applied to various other procedures that utilize non-optical visualization of an instrument or tool. Some example applications include infrared guided IV line or catheter insertion, or insertion of hardware or a needle using x-ray or fluoroscopic guidance or any other application wherein the proceduralist uses a non-optical imaging device to guide an instrument, needle, or a piece of hardware into the body. In such cases, the instrument, needle, or hardware may not be visible to the user once subcutaneous. The device may allow the proceduralist to understand the 3-dimensional relationship of the needle or instrument relative to a known anatomic structure, as identified by the non-optical imaging device.

As discussed above, some embodiments of the present disclosure include systems, methods, and devices for virtually visualizing the 3-dimensional position of an endotracheal tube or instrument in relation to a video laryngoscope camera or a non-optical imaging device that overcomes many of the limitations of the prior art. Such techniques can be applied to several applications, including use of video laryngoscope or non-optical imaging device (such as an infrared camera, or x-ray generator). Additionally, the device may serve as an airway assessment and management tool that allows a video laryngoscope to receive information regarding the location of the trachea when used in conjunction with an associated neck apparatus.

1. A system for facilitating an endotracheal intubation, the system comprising:
a video laryngoscope apparatus including:
a video camera configured to capture video images; and
one or more magnetometers configured to generate magnetometer signals based on an interaction with one or more magnetic elements associated with an intubation device;
a processor communicatively coupled to the one or more magnetometers and configured to:
receive the magnetometer signals; and
calculate position information regarding the intubation device relative to the video laryngoscope apparatus; and
an information output device communicatively coupled to the processor and configured to output information to a user indicating or based on the calculated position information regarding the intubation device.

2. The system of claim 1, wherein the information output device of the intubation device guidance system comprises a visual display device including one or more light-emitting diodes (LEDs) or other visual elements.

3. The system of claim 1, wherein:
the intubation device comprises a styletted endotracheal tube including a stylet configured to be arranged within a flexible endotracheal tube; and
the one or more magnetic elements associated with an intubation device comprise one or more magnetic portions of the stylet or one or more magnets secured to the stylet.

4. The system of claim 1, wherein the processor is configured to:
determine, based on the magnetometer signals, intubation guidance information including at least one of (a) a spatial location of the intubation device, (b) a proximity of the intubation device relative to the video laryngoscope apparatus, or (b) a safety metric regarding the intubation device; and
control the information output device to display or otherwise output the intubation guidance information.

5. The system of claim 1, wherein the processor of the intubation device guidance system is configured to:
determine, based on the magnetometer signals, that the intubation device has advanced to a reference point, axis, or plane associated with a field of view of the video camera; and
in response to determining that the intubation device has advanced to the reference point, axis, or plane associated with a field of view of the video camera, output a notification via the information output device indicating that the user can switch attention to a video display configured to display video images captured by the video camera of the video laryngoscope apparatus.

6. The system of claim 1, further comprising a machine vision system configured to:
receive video images captured by the video camera, the video images corresponding with a field of view of the video camera;
analyze the received video images to identify the intubation device in the field of view of the video camera; and
in response to identifying the intubation device in the field of view of the video camera, output a notification via the information output device.

7. A system for facilitating an endotracheal intubation, the system comprising:
a video laryngoscope apparatus including a video laryngoscope body and a video camera arranged near an end of the video laryngoscope body and configured to capture video images; and
an intubation device guidance system including:
one or more magnetometers arranged relative to the video laryngoscope body and configured to generate magnetometer signals based on an interaction with one or more magnetic elements associated with an intubation device;
a processor communicatively coupled to the one or more magnetometers and configured to:
receive the magnetometer signals; and
calculate position information regarding the intubation device relative to the video laryngoscope apparatus; and
an information output device communicatively coupled to the processor and configured to output information to a user indicating or based on the calculated position information regarding the intubation device.

8. The system of claim 7, wherein the intubation device guidance system is physically distinct from the video laryngoscope apparatus.

9. The system of claim 7, wherein the intubation device guidance system is physically integrated with the video laryngoscope apparatus.

10. The system of claim 7, wherein:
a portion of the video laryngoscope body is configured to be inserted in a laryngoscope blade; and
the one or more magnetometers are provided on an intubation guidance system element configured to be arranged at least partially within the laryngoscope blade.

11. The system of claim 10, wherein the intubation device guidance system element is configured to be secured or arranged on or adjacent an outer surface of the video laryngoscope body.

12. The system of claim 7, wherein the information output device of the intubation device guidance system comprises a visual display device including one or more light-emitting diodes (LEDs) or other visual elements.

13. The system of claim 7, wherein:
the intubation device comprises a styletted endotracheal tube including a stylet configured to be arranged within a flexible endotracheal tube; and
the one or more magnetic elements associated with an intubation device comprise one or more magnetic portions of the stylet or one or more magnets secured to the stylet.

14. The system of claim 7, wherein the processor of the intubation device guidance system is configured to:
determine, based on the magnetometer signals, intubation guidance information including at least one of (a) a spatial location of the intubation device, (b) a proximity of the intubation device relative to the video laryngoscope apparatus, or (b) a safety metric regarding the intubation device; and
control the information output device to display or otherwise output the intubation guidance information.

15. The system of claim 7, wherein the processor of the intubation device guidance system is configured to:
determine, based on the magnetometer signals, that the intubation device has advanced beyond a predefined reference point, axis, or plane associated with the video laryngoscope apparatus; and
control the information output device to display or otherwise provide an indication that the intubation device has advanced beyond the predefined reference point, axis, or plane.

16. The system of claim 7, wherein the processor of the intubation device guidance system is configured to:
determine, based on the magnetometer signals, that the intubation device has advanced to a reference point, axis, or plane associated with a field of view of the video camera; and
in response to determining that the intubation device has advanced to the reference point, axis, or plane associated with a field of view of the video camera, output a notification via the information output device indicating that the user can switch attention to a video display configured to display video images captured by the video camera of the video laryngoscope apparatus.

17. The system of claim 7, further comprising a machine vision system configured to:
receive video images captured by the video camera, the video images corresponding with a field of view of the video camera;
analyze the received video images to identify the intubation device in the field of view of the video camera; and
in response to identifying the intubation device in the field of view of the video camera, output a notification via the information output device.

18. The system of claim 7, wherein the intubation device guidance includes only a single magnetometer.

19. An intubation guidance apparatus for facilitating an intubation, the intubation guidance apparatus comprising:
one or more non-optical sensors configured to generate non-optical sensor signals based on interactions with one or more detectable elements associated with an intubation device; and
a processor coupled to the one or more non-optical sensors magnetometers and configured to:
receive the non-optical sensor;
determine position information regarding the intubation device based on the non-optical sensor signals;
generate intubation guidance information based on the determined position information regarding the intubation device; and
communicate the intubation guidance information for output to a user via an information output device.

20. The intubation guidance apparatus of claim 19, wherein:
the intubation device comprises a styletted endotracheal tube including a stylet configured to be arranged within a flexible endotracheal tube;
the one or more detectable elements comprise one or more magnetic elements associated with the styletted endotracheal tube; and
the one or more non-optical sensors comprise one or more magnetometers configured to detect the one or more magnetic elements associated with the styletted endotracheal tube.
